SEQUENCE LISTING (5 sequences)

SEQ ID NO: 1 - DNA, 15111 bp, Human respiratory syncytial virus
  source 1..15111: mol_type = other DNA
acgggaaaaaatgcgtacaacaaacttgcataaaccaaaaaaatggggcaaataagaatttgataagtaccacttaaatttaactcccttggttagagatgggcagcaattcattgagtatgataaaagttagattacaaaatttgtttgacaatgatgaagtagcattgttaaaaataacatgctatactgataaattaatacatttaactaatgctttggctaaggcagtgatacatacaatcaaattgaatggcattgtgtttgtgcatgttattacaagtagtgatatttgccctaataataatattgtagtaaaatccaatttcacaacaatgccagtactacaaaatggaggttatatatgggaaatgatggaattaacacattgctctcaacctaatggtctactagatgacaattgtgaaattaaattctccaaaaaactaagtgattcaacaatgaccaattatatgaatcaattatctgaattacttggatttgatcttaatccataaattataattaatatcaactagcaaatcaatgtcactaacaccattagttaatataaaacttaacagaagacaaaaatggggcaaataaatcaattcagccaacccaaccatggacacaacccacaatgataatacaccacaaagactgatgatcacagacatgagaccgttgtcacttgagaccataataacatcactaaccagagacatcataacacacaaatttatatacttgataaatcatgaatgcatagtgagaaaacttgatgaaagacaggccacatttacattcctggtcaactatgaaatgaaactattacacaaagtaggaagcactaaatataaaaaatatactgaatacaacacaaaatatggcactttccctatgccaatattcatcaatcatgatgggttcttagaatgcattggcattaagcctacaaagcatactcccataatatacaagtatgatctcaatccataaatttcaacacaatattcacacaatctaaaacaacaactctatgcataactatactccatagtccagatggagcctgaaaattatagtaatttaaaacttaaggagagatataagatagaagatggggcaaatacaaccatggctcttagcaaagtcaagttgaatgatacactcaacaaagatcaacttctgtcatccagcaaatacaccatccaacggagcacaggagatagtattgatactcctaattatgatgtgcagaaacacatcaataagttatgtggcatgttattaatcacagaagatgctaatcataaattcactgggttaataggtatgttatatgcgatgtctaggttaggaagagaagacaccataaaaatactcagagatgcgggatatcatgtaaaagcaaatggagtagatgtaacaacacatcgtcaagacattaatggaaaagaaatgaaatttgaagtgttaacattggcaagcttaacaactgaaattcaaatcaacattgagatagaatctagaaaatcctacaaaaaaatgctaaaagaaatgggagaggtagctccagaatacaggcatgactctcctgattgtgggatgataatattatgtatagcagcattagtaataactaaattagcagcaggggacagatctggtcttacagccgtgattaggagagctaataatgtcctaaaaaatgaaatgaaacgttacaaaggcttactacccaaggacatagccaacagcttctatgaagtgtttgaaaaacatccccactttatagatgtttttgttcattttggtatagcacaatcttctaccagaggtggcagtagagttgaagggatttttgcaggattgtttatgaatgcctatggtgcagggcaagtgatgttacggtggggagtcttagcaaaatcggttaaaaatattatgttaggacatgctagtgtgcaagcagaaatggaacaagttgttgaggtttatgaatatgcccaaaaattgggtggtgaagcaggattctaccatatattgaacaacccaaaagcatcattattatctttgactcaatttcctcacttctccagtgtagtattaggcaatgctgctggcctaggcataatgggagagtacagaggtacaccgaggaatcaagatctatatgatgcagcaaaggcatatgctgaacaactcaaagaaaatggtgtgattaactacagtgtactagacttgacagcagaagaactagaggctatcaaacatcagcttaatccaaaagataatgatgtagagctttgagttaataaaaaatggggcaaataaatcatcatggaaaagtttgctcctgaattccatggagaagatgcaaacaacagggctactaaattcctagaatcaataaagggcaaattcacatcacccaaagatcccaagaaaaaagatagtatcatatctgtcaactcaatagatatagaagtaaccaaagaaagccctataacatcaaattcaactattatcaacccaacaaatgagacagatgatactgcagggaacaagcccaattatcaaagaaaacctctagtaagtttcaaagaagaccctacaccaagtgataatcccttttctaaactatacaaagaaaccatagaaacatttgataacaatgaagaagaatccagctattcatacgaagaaataaatgatcagacaaacgataatataacagcaagattagataggattgatgaaaaattaagtgaaatactaggaatgcttcacacattagtagtggcaagtgcaggacctacatctgctcgggatggtataagagatgccatggttggtttaagagaagaaatgatagaaaaaatcagaactgaagcattaatgaccaatgacagattagaagctatggcaagactcaggaatgaggaaagtgaaaagatggcaaaagacacatcagatgaagtgtctctcaatccaacatcagagaaattgaacaacctattggaagggaatgatagtgacaatgatctatcacttgaagatttctgattagttaccaatcttcacatcaacacacaataccaacagaagaccaacaaactaaccaacccaatcatccaaccaaacatccatccgccaatcagccaaacagccaacaaaacaaccagccaatccaaaactaaccacccggaaaaaatctataatatagttacaaaaaaaggaaagggtggggcaaatatggaaacatacgtgaacaagcttcacgaaggctccacatacacagctgctgttcaatacaatgtcttagaaaaagacgatgaccctgcatcacttacaatatgggtgcccatgttccaatcatctatgccagcagatttacttataaaagaactagctaatgtcaacatactagtgaaacaaatatccacacccaagggaccttcactaagagtcatgataaactcaagaagtgcagtgctagcacaaatgcccagcaaatttaccatatgcgctaatgtgtccttggatgaaagaagcaaactagcatatgatgtaaccacaccctgtgaaatcaaggcatgtagtctaacatgcctaaaatcaaaaaatatgttgactacagttaaagatctcactatgaagacactcaaccctacacatgatattattgctttatgtgaatttgaaaacatagtaacatcaaaaaaagtcataataccaacatacctaagatccatcagtgtcagaaataaagatctgaacacacttgaaaatataacaaccactgaattcaaaaatgctatcacaaatgcaaaaatcatcccttactcaggattactattagtcatcacagtgactgacaacaaaggagcattcaaatacataaagccacaaagtcaattcatagtagatcttggagcttacctagaaaaagaaagtatatattatgttaccacaaattggaagcacacagctacacgatttgcaatcaaacccatggaagattaacctttttcctctacatcagtgtgttaattcatacaaactttctacctacattcttcacttcaccatcacaatcacaaacactctgtggttcaaccaatcaaacaaaacttatctgaagtcccagatcatcccaagtcattgtttatcagatctagtactcaaataagttaataaaaaatatacacatggggcaaataatcattggaggaaatccaactaatcacaatatctgttaacatagacaagtccacacaccatacagaatcaaccaatggaaaatacatccataacaatagaattctcaagcaaattctggccttactttacactaatacacatgatcacaacaataatctctttgctaatcataatctccatcatgattgcaatactaaacaaactttgtgaatataacgtattccataacaaaacctttgagttaccaagagctcgagttaatacttgataaagtagttaattaaaaatagtcataacaatgaactaggatatcaagactaacaataacattggggcaaatgcaaacatgtccaaaaacaaggaccaacgcaccgctaagacattagaaaggacctgggacactctcaatcatttattattcatatcatcgtgcttatataagttaaatcttaaatctgtagcacaaatcacattatccattctggcaatgataatctcaacttcacttataattgcagccatcatattcatagcctcggcaaaccacaaagtcacaccaacaactgcaatcatacaagatgcaacaagccagatcaagaacacaaccccaacatacctcacccagaatcctcagcttggaatcagtccctctaatccgtctgaaattacatcacaaatcaccaccatactagcttcaacaacaccaggagtcaagtcaaccctgcaatccacaacagtcaagaccaaaaacacaacaacaactcaaacacaacccagcaagcccaccacaaaacaacgccaaaacaaaccaccaagcaaacccaataatgattttcactttgaagtgttcaactttgtaccctgcagcatatgcagcaacaatccaacctgctgggctatctgcaaaagaataccaaacaaaaaaccaggaaagaaaaccactaccaagcccacaaaaaaaccaaccctcaagacaaccaaaaaagatcccaaacctcaaaccactaaatcaaaggaagtacccaccaccaagcccacagaagagccaaccatcaacaccaccaaaacaaacatcataactacactactcacctccaacaccacaggaaatccagaactcacaagtcaaatggaaaccttccactcaacttcctccgaaggcaatccaagcccttctcaagtctctacaacatccgagtacccatcacaaccttcatctccacccaacacaccacgccagtagttacttaaaaacatattatcacaaaaggccttgaccaacttaaacagaatcaaaataaactctggggcaaataacaatggagttgctaatcctcaaagcaaatgcaattaccacaatcctcactgcagtcacattttgttttgcttctggtcaaaacatcactgaagaattttatcaatcaacatgcagtgcagttagcaaaggctatcttagtgctctgagaactggttggtataccagtgttataactatagaattaagtaatatcaagaaaaataagtgtaatggaacagatgctaaggtaaaattgataaaacaagaattagataaatataaaaatgctgtaacagaattgcagttgctcatgcaaagcacacaagcaacaaacaatcgagccagaagagaactaccaaggtttatgaattatacactcaacaatgccaaaaaaaccaatgtaacattaagcaagaaaaggaaaagaagatttcttggttttttgttaggtgttggatctgcaatcgccagtggcgttgctgtatctaaggtcctgcacctagaaggggaagtgaacaagatcaaaagtgctctactatccacaaacaaggctgtagtcagcttatcaaatggagttagtgttttaaccagcaaagtgttagacctcaaaaactatatagataaacaattgttacctattgtgaacaagcaaagctgcagcatatcaaatatagaaactgtgatagagttccaacaaaagaacaacagactactagagattaccagggaatttagtgttaatgcaggcgtaactacacctgtaagcacttacatgttaactaatagtgaattattgtcattaatcaatgatatgcctataacaaatgatcagaaaaagttaatgtccaacaatgttcaaatagttagacagcaaagttactctatcatgtccataataaaagaggaagtcttagcatatgtagtacaattaccactatatggtgttatagatacaccctgttggaaactacacacatcccctctatgtacaaccaacacaaaagaagggtccaacatctgtttaacaagaactgacagaggatggtactgtgacaatgcaggatcagtatctttcttcccacaagctgaaacatgtaaagttcaatcaaatcgagtattttgtgacacaatgaacagtttaacattaccaagtgaagtaaatctctgcaatgttgacatattcaaccccaaatatgattgtaaaattatgacttcaaaaacagatgtaagcagctccgttatcacatctctaggagccattgtgtcatgctatggcaaaactaaatgtacagcatccaataaaaatcgtggaatcataaagacattttctaacgggtgcgattatgtatcaaataaaggggtggacactgtgtctgtaggtaacacattatattatgtaaataagcaagaaggtaaaagtctctatgtaaaaggtgaaccaataataaatttctatgacccattagtattcccctctgatgaatttgatgcatcaatatctcaagtcaacgagaagattaaccagagcctagcatttattcgtaaatccgatgaattattacataatgtaaatgctggtaaatccaccacaaatatcatgataactactataattatagtgattatagtaatattgttatcattaattgctgttggactgctcttatactgtaaggccagaagcacaccagtcacactaagcaaagatcaactgagtggtataaataatattgcatttagtaactaaataaaaatagcacctaatcatgttcttacaatggtttactatctgctcatagacaacccatctgtcattggattttcttaaaatctgaacttcatcgaaactctcatctataaaccatctcacttacactatttaagtagattcctagtttatagttatataaaacacaattgcatgccagattaacttaccatctgtaaaaatgaaaactggggcaaatatgtcacgaaggaatccttgcaaatttgaaattcgaggtcattgcttaaatggtaagaggtgtcattttagtcataattattttgaatggccaccccatgcactgcttgtaagacaaaactttatgttaaacagaatacttaagtctatggataaaagtatagataccttatcagaaataagtggagctgcagagttggacagaacagaagagtatgctcttggtgtagttggagtgctagagagttatataggatcaataaacaatataactaaacaatcagcatgtgttgccatgagcaaactcctcactgaactcaatagtgatgatatcaaaaagctgagggacaatgaagagctaaattcacccaagataagagtgtacaatactgtcatatcatatattgaaagcaacaggaaaaacaataaacaaactatccatctgttaaaaagattgccagcagacgtattgaagaaaaccatcaaaaacacattggatatccataagagcataaccatcaacaacccaaaagaatcaactgttagtgatacaaatgaccatgccaaaaataatgatactacctgacaaatatccttgtagtataacttccatactaataacaagtagatgtagagttactatgtataatcaaaagaacacactatatttcaatcaaaacaacccaaataaccatatgtactcaccgaatcaaacattcaatgaaatccattggacctctcaagaattgattgacacaattcaaaattttctacaacatctaggtattattgaggatatatatacaatatatatattagtgtcataacactcaattctaacactcaccacatcgttacattattaattcaaacaattcaagttgtgggacaaaatggatcccattattaatggaaattctgctaatgtttatctaaccgatagttatttaaaaggtgttatctctttctcagagtgtaatgctttaggaagttacatattcaatggtccttatctcaaaaatgattataccaacttaattagtagacaaaatccattaatagaacacatgaatctaaagaaactaaatataacacagtccttaatatctaagtatcataaaggtgaaataaaattagaagaacctacttattttcagtcattacttatgacatacaagagtatgacctcgtcagaacagattgctaccactaatttacttaaaaagataataagaagagctatagaaataagtgatgtcaaagtctatgctatattgaataaactagggcttaaagaaaaggacaagattaaatccaacaatggacaagatgaagacaactcagttattacgaccataatcaaagatgatatactttcagctgttaaagataatcaatctcatcttaaagcagacaaaaatcactctacaaaacaaaaagacacaatcaaaacaacactcttgaagaaattgatgtgttcaatgcaacatcctccatcatggttaatacattggtttaacttatacacaaaattaaacaacatattaacacagtatcgatcaaatgaggtaaaaaaccatgggtttacattgatagataatcaaactcttagtggatttcaatttattttgaaccaatatggttgtatagtttatcataaggaactcaaaagaattactgtgacaacctataatcaattcttgacatggaaagatattagccttagtagattaaatgtttgtttaattacatggattagtaactgcttgaacacattaaataaaagcttaggcttaagatgcggattcaataatgttatcttgacacaactattcctttatggagattgtatactaaagctatttcacaatgaggggttctacataataaaagaggtagagggatttattatgtctctaattttaaatataacagaagaagatcaattcagaaaacgattttataatagtatgctcaacaacatcacagatgctgctaataaagctcagaaaaatctgctatcaagagtatgtcatacattattagataagacagtgtccgataatataataaatggcagatggataattctattaagtaagttccttaaattaattaagcttgcaggtgacaataaccttaacaatctgagtgaactatattttttgttcagaatatttggacacccaatggtagatgaaagacaagccatggatgctgttaaaattaattgcaatgagaccaaattttacttgttaagcagtctgagtatgttaagaggtgcctttatatatagaattataaaagggtttgtaaataattacaacagatggcctactttaagaaatgctattgttttacccttaagatggttaacttactataaactaaacacttatccttctttgttggaacttacagaaagagatttgattgtgttatcaggactacgtttctatcgtgagtttcggttgcctaaaaaagtggatcttgaaatgattataaatgataaagctatatcacctcctaaaaatttgatatggactagtttccctagaaattacatgccatcacacatacaaaactatatagaacatgaaaaattaaaattttccgagagtgataaatcaagaagagtattagagtattatttaagagataacaaattcaatgaatgtgatttatacaactgtgtagttaatcaaagttatctcaacaaccctaatcatgtggtatcattgacaggcaaagaaagagaactcagtgtaggtagaatgtttgcaatgcaaccgggaatgttcagacaggttcaaatattggcagagaaaatgatagctgaaaacattttacaattctttcctgaaagtcttacaagatatggtgatctagaactacaaaaaatattagaactgaaagcaggaataagtaacaaatcaaatcgctacaatgataattacaacaattacattagtaagtgctctatcatcacagatctcagcaaattcaatcaagcatttcgatatgaaacgtcatgtatttgtagtgatgtgctggatgaactgcatggtgtacaatctctattttcctggttacatttaactattcctcatgtcacaataatatgcacatataggcatgcacccccctatataggagatcatattgtagatcttaacaatgtagatgaacaaagtggattatatagatatcacatgggtggcatcgaagggtggtgtcaaaaactatggaccatagaagctatatcactattggatctaatatctctcaaagggaaattctcaattactgctttaattaatggtgacaatcaatcaatagatataagcaaaccaatcagactcatggaaggtcaaactcatgctcaagcagattatttgctagcattaaatagccttaaattactgtataaagagtatgcaggcataggccacaaattaaaaggaactgagacttatatatcacgagatatgcaatttatgagtaaaacaattcaacataacggtgtatattacccagctagtataaagaaagtcctaagagtgggaccgtggataaacactatacttgatgatttcaaagtgagtctagaatctataggtagtttgacacaagaattagaatatagaggtgaaagtctattatgcagtttaatatttagaaatgtatggttatataatcagattgctctacaattaaaaaatcatgcattatgtaacaataaactatatttggacatattaaaggttctgaaacacttaaaaaccttttttaatcttgataatattgatacagcattaacattgtatatgaatttacccatgttatttggtggtggtgatcccaacttgttatatcgaagtttctatagaagaactcctgacttcctcacagaggctatagttcactctgtgttcatacttagttattatacaaaccatgacttaaaagataaacttcaagatctgtcagatgatagattgaataagttcttaacatgcataatcacgtttgacaaaaaccctaatgctgaattcgtaacattgatgagagatcctcaagctttagggtctgagagacaagctaaaattactagcgaaatcaatagactggcagttacagaggttttgagtacagctccaaacaaaatattctccaaaagtgcacaacattatactactacagagatagatctaaatgatattatgcaaaatatagaacctacatatcctcatgggctaagagttgtttatgaaagtttacccttttataaagcagagaaaatagtaaatcttatatcaggtacaaaatctataactaacatactggaaaaaacttctgccatagacttaacagatattgatagagccactgagatgatgaggaaaaacataactttgcttataaggatacttccattggattgtaacagagataaaagagagatattgagtatggaaaacctaagtattactgaattaagcaaatatgttagggaaagatcttggtctttatccaatatagttggtgttacatcacccagtatcatgtatacaatggacatcaaatatactacaagcactatatctagtggcataattatagagaaatataatgttaacagtttaacacgtggtgagagaggacccactaaaccatgggttggttcatctacacaagagaaaaaaacaatgccagtttataatagacaagtcttaaccaaaaaacagagagatcaaatagatctattagcaaaattggattgggtgtatgcatctatagataacaaggatgaattcatggaagaactcagcataggaacccttgggttaacatatgaaaaggccaagaaattatttccacaatatttaagtgtcaattatttgcatcgccttacagtcagtagtagaccatgtgaattccctgcatcaataccagcttatagaacaacaaattatcactttgacactagccctattaatcgcatattaacagaaaagtatggtgatgaagatattgacatagtattccaaaactgtataagctttggccttagtttaatgtcagtagtagaacaatttactaatgtatgtcctaacagaattattctcatacctaagcttaatgagatacatttgatgaaacctcccatattcacaggtgatgttgatattcacaagttaaaacaagtgatacaaaaacagcatatgtttttaccagacaaaataagtttgactcaatatgtggaattattcttaagtaataaaacactcaaatctggatctcatgttaattctaatttaatattggcacataaaatatctgactattttcataatacttacattttaagtactaatttagctggacattggattctgattatacaacttatgaaagattctaaaggtatttttgaaaaagattggggagagggatatataactgatcatatgtttattaatttgaaagttttcttcaatgcttataagacctatctcttgtgttttcataaaggttatggcaaagcaaagctggagtgtgatatgaacacttcagatcttctatgtgtattggaattaatagacagtagttattggaagtctatgtctaaggtatttttagaacaaaaagttatcaaatacattcttagccaagatgcaagtttacatagagtaaaaggatgtcatagcttcaaattatggtttcttaaacgtcttaatgtagcagaattcacagtttgcccttgggttgttaacatagattatcatccaacacatatgaaagcaatattaacttatatagatcttgttagaatgggattgataaatatagatagaatacacattaaaaataaacacaaattcaatgatgaattttatacttctaatctcttctacattaattataacttctcagataatactcatctattaactaaacatataaggattgctaattctgaattagaaaataattacaacaaattatatcatcctacaccagaaaccctagagaatatactagccaatccgattaaaagtaatgacaaaaagacactgaatgactattgtataggtaaaaatgttgactcaataatgttaccattgttatctaataagaagcttattaaatcgtctgcaatgattagaaccaattacagcaaacaagatttgtataatttattccctatggttgtgattgatagaattatagatcattcaggcaatacagccaaatccaaccaactttacactactacttcccaccaaatatccttagtgcacaatagcacatcactttactgcatgcttccttggcatcatattaatagattcaattttgtatttagttctacaggttgtaaaattagtatagagtatattttaaaagatcttaaaattaaagatcccaattgtatagcattcataggtgaaggagcagggaatttattattgcgtacagtagtggaacttcatcctgacataagatatatttacagaagtctgaaagattgcaatgatcatagtttacctattgagtttttaaggctgtacaatggacatatcaacattgattatggtgaaaatttgaccattcctgctacagatgcaaccaacaacattcattggtcttatttacatataaagtttgctgaacctatcagtctttttgtctgtgatgccgaattgtctgtaacagtcaactggagtaaaattataatagaatggagcaagcatgtaagaaagtgcaagtactgttcctcagttaataaatgtatgttaatagtaaaatatcatgctcaagatgatattgatttcaaattagacaatataactatattaaaaacttatgtatgcttaggcagtaagttaaagggatcggaggtttacttagtccttacaataggtcctgcgaatatattcccagtatttaatgtagtacaaaatgctaaattgatactatcaagaaccaaaaatttcatcatgcctaagaaagctgataaagagtctattgatgcaaatattaaaagtttgataccctttctttgttaccctataacaaaaaaaggaattaatactgcattgtcaaaactaaagagtgttgttagtggagatatactatcatattctatagctggacgtaatgaagttttcagcaataaacttataaatcataagcatatgaacatcttaaaatggttcaatcatgttttaaatttcagatcaacagaactaaactataaccatttatatatggtagaatctacatatccttacctaagtgaattgttaaacagcttgacaaccaatgaacttaaaaaactgattaaaatcacaggtagtctgttatacaactttcataatgaataatgaataaagatcttataataaaaattcccatagctatacactaacactgtattcaattatagttattaaaaattaaaaatcatataattttttaaataacttttagtgaactaatcctaaagttatcattttaatcttggaggaataaatttaaaccctaatctaattggtttatatgtgtattaactaaattacgagatattagtttttgacactttttttctcgt

SEQ ID NO: 2 - DNA, 15111 bp, Human respiratory syncytial virus
  source 1..15111: mol_type = other DNA
acgggaaaaaatgcgtacaacaaacttgcataaaccaaaaaaatggggcaaataagaatttgataagtaccacttaaatttaactcccttggttagagatgggcagcaattcattgagtatgataaaagtcagattgcaaaatctattcgataatgacgaagtggcactattaaaaattacatgttataccgataaattgatacatctaactaatgcattagctaaagctgtaatacatacaattaaacttaatggaatagtgtttgtacatgtaattacatctagtgatatatgccctaataataatatcgtagtcaagtctaattttacaacaatgccagtgttacaaaatggcggatatatttgggaaatgatggaattgacacattgctcacaacctaatggtctattagacgataattgcgaaattaaatttagtaagaaattatccgatagtacaatgactaattatatgaatcaattatccgaattgttaggtttcgatcttaatccataaattataattaatatcaactagcaaatcaatgtcactaacaccattagttaatataaaacttaacagaagacaaaaatggggcaaataaatcaattcagccaacccaaccatggacacaacccacaatgataatacaccacaaagactgatgattaccgatatgagaccgttgtcacttgagacaattataactagcctaactagagatataataacacataaatttatatatctgattaatcacgaatgcatcgtgaggaaattggacgaaagacaggccacatttacattcttagtcaattacgaaatgaaactattgcataaggtaggctcaactaagtataagaaatataccgaatataacactaaatacggaacattcccaatgcctatattcataaatcacgacgggtttctcgaatgcataggcataaaacctacaaaacatacacccataatctataaatacgatcttaacccataaatttcaacacaatattcacacaatctaaaacaacaactctatgcataactatactccatagtccagatggagcctgaaaattatagtaatttaaaacttaaggagagatataagatagaagatggggcaaatacaaccatggctcttagcaaagtcaagttgaatgatacattgaataaagatcaattactatctagctcgaaatatactatccaacggtctacaggcgattcaatagatacacctaattacgatgtgcaaaaacatattaataaattgtgtggtatgttattgattaccgaagacgcaaatcataaatttacagggttaatcggtatgttatacgctatgtctagattaggtagggaagatacaattaaaatacttagagacgcaggatatcacgttaaagctaacggagtagacgtaactacacatagacaggatattaacggtaaggaaatgaaattcgaagtgttaacactcgctagcttaactaccgaaatacaaattaatatcgaaatcgaatcacgtaaatcttataagaaaatgcttaaagaaatgggcgaagtcgcacccgaatatagacacgatagtcccgattgtggtatgattatactatgtatagccgcattagtgataactaagttggccgcaggcgatagatccggattaaccgcagtgatacgtagagcgaataacgtacttaaaaacgaaatgaaacggtataagggtctattaccaaaagatatagcgaatagtttttacgaagtattcgaaaaacatccacattttatagacgtttttgtgcatttcggaatcgcacaatctagtactagaggaggatctagggttgagggtatattcgcaggattgtttatgaacgcatacggagcaggtcaagtcatgcttagatggggagtactcgcaaaatccgttaaaaatattatgttaggacacgctagcgtacaagccgaaatggaacaagtcgttgaggtatacgaatacgcacaaaaattaggtggagaagcaggattttatcatatactgaataatcctaaagctagtctattaagcttaacacaatttccacatttttctagcgtagtgttaggtaacgcagctggcctaggcataatgggcgaatataggggtacacctagaaatcaggatctatatgacgcagctaaagcatacgctgaacaattgaaagagaatggagtgataaattattccgtactcgatctaacagccgaagagttggaggcaattaaacatcaattgaatccgaaagataatgacgttgagttgtgagttaataaaaaatggggcaaataaatcatcatggaaaagtttgctcctgaattccatggagaagacgcaaataatagggcaacaaaattcttagagtcaatcaagggtaagtttacaagtccaaaagatccaaagaagaaagatagtataataagcgtaaactcaattgatatcgaggtgacaaaggaatcacctataacatctaatagtacaataataaatcccactaacgaaacagacgataccgcaggcaataaacctaattatcaacggaaacccttagtgtcattcaaagaagatccaacacctagtgataatccctttagtaaattgtataaggaaacaatcgaaacattcgataataacgaagaagaatcatcatactcatacgaagagataaacgatcagactaacgataatataaccgctagactagatagaatagacgaaaaactatctgaaatactaggtatgttacacacactagtagtcgcatctgccggacctacaagtgctagagatgggataagggatgcaatggtagggttaagggaagaaatgatagagaaaattagaaccgaagcattaatgactaacgatagactcgaagcaatggctagacttagaaacgaagaatccgaaaagatggcaaaagatacatctgacgaagtgtcacttaatcctactagcgaaaaattgaataatctattagagggaaacgatagtgataacgatctatcactcgaagatttctgattagttaccaatcttcacatcaacacacaataccaacagaagaccaacaaactaaccaacccaatcatccaaccaaacatccatccgccaatcagccaaacagccaacaaaacaaccagccaatccaaaactaaccacccggaaaaaatctataatatagttacaaaaaaaggaaagggtggggcaaatatggaaacatacgtgaacaagcttcacgaaggatcaacatatacagctgcagtccaatataacgtactcgaaaaagacgacgatcccgctagcctaacaatatgggtcccaatgtttcaatctagtatgcccgctgatctattaatcaaagaactagctaacgttaacatactagtcaaacaaattagtacacctaagggaccctcacttagagtgatgattaatagtagatccgcagtcctagcacaaatgcctagtaagtttacaatatgtgctaacgtaagcttagacgaacgatcaaaactagcatacgatgtgacaacaccatgcgaaatcaaagcatgttcattgacatgtcttaaatcaaagaatatgctaacaacagtcaaagatctaacaatgaaaacacttaatcccacacacgatataatcgcactatgcgaattcgaaaatatagtgactagtaagaaagtgataatccctacataccttagatcaatatccgttagaaataaggatctgaatacactcgaaaatataacaacaaccgaattcaaaaacgctataactaacgctaagataatcccttactccggactattgttagtgataaccgtaaccgataataagggagcattcaaatacataaaaccccaatcccaatttatagtcgatttaggcgcatacttagaaaaagaatcaatctattacgttacaactaattggaaacataccgctactagattcgcaatcaaacctatggaagattaacctttttcctctacatcagtgtgttaattcatacaaactttctacctacattcttcacttcaccatcacaatcacaaacactctgtggttcaaccaatcaaacaaaacttatctgaagtcccagatcatcccaagtcattgtttatcagatctagtactcaaataagttaataaaaaatatacacatggggcaaataatcattggaggaaatccaactaatcacaatatctgttaacatagacaagtccacacaccatacagaatcaaccaatggaaaatacatccataacaatagaattctctagcaaattttggccttactttacactaatacacatgataactacaatcatatccctattaatcataatctcaattatgatcgcaatccttaacaaactatgtgagtataacgtattccataacaaaacattcgaattgccaagagctcgagtgaatacctgataaagtagttaattaaaaatagtcataacaatgaactaggatatcaagactaacaataacattggggcaaatgcaaacatgtccaaaaacaaggaccaacgcaccgctaagacattagaaaggacctgggacactctcaatcatttattattcatatcatcgtgcttatataagttaaatcttaaatctgtagcacaaatcacattatccattctggcaatgataatctcaacttcacttataattgcagccatcatattcatagcctcggcaaaccacaaagtcacaccaacaactgcaatcatacaagatgcaacaagccagatcaagaacacaaccccaacatacctcacccagaatcctcagcttggaatcagtccctctaatccgtctgaaattacatcacaaatcaccaccatactagcttcaacaacaccaggagtcaagtcaaccctgcaatccacaacagtcaagaccaaaaacacaacaacaactcaaacacaacccagcaagcccaccacaaaacaacgccaaaacaaaccaccaagcaaacccaataatgattttcactttgaagtgttcaactttgtaccctgcagcatatgcagcaacaatccaacctgctgggctatctgcaaaagaataccaaacaaaaaaccaggaaagaaaaccactaccaagcccacaaaaaaaccaaccctcaagacaaccaaaaaagatcccaaacctcaaaccactaaatcaaaggaagtacccaccaccaagcccacagaagagccaaccatcaacaccaccaaaacaaacatcataactacactactcacctccaacaccacaggaaatccagaactcacaagtcaaatggaaaccttccactcaacttcctccgaaggcaatccaagcccttctcaagtctctacaacatccgagtacccatcacaaccttcatctccacccaacacaccacgccagtagttacttaaaaacatattatcacaaaaggccttgaccaacttaaacagaatcaaaataaactctggggcaaataacaatggagttgctaatcctcaaagcaaatgcaattaccacaatcctcactgcagtcacattttgttttgcttctggtcaaaacatcactgaagaattttatcaatcaacatgcagtgcagttagcaaaggctatcttagtgctctgagaactggttggtataccagtgttataactatagaattaagtaatatcaagaaaaataagtgtaatggaacagatgctaaggtaaaattgataaaacaagaattagataaatataaaaatgctgtaacagaattgcagttgctcatgcaaagcacacaagcaacaaacaatcgagccagaagagaactaccaaggtttatgaattatacactcaacaatgccaaaaaaaccaatgtaacattaagcaagaaaaggaaaagaagatttcttggttttttgttaggtgttggatctgcaatcgccagtggcgttgctgtatctaaggtcctgcacctagaaggggaagtgaacaagatcaaaagtgctctactatccacaaacaaggctgtagtcagcttatcaaatggagttagtgttttaaccagcaaagtgttagacctcaaaaactatatagataaacaattgttacctattgtgaacaagcaaagctgcagcatatcaaatatagaaactgtgatagagttccaacaaaagaacaacagactactagagattaccagggaatttagtgttaatgcaggcgtaactacacctgtaagcacttacatgttaactaatagtgaattattgtcattaatcaatgatatgcctataacaaatgatcagaaaaagttaatgtccaacaatgttcaaatagttagacagcaaagttactctatcatgtccataataaaagaggaagtcttagcatatgtagtacaattaccactatatggtgttatagatacaccctgttggaaactacacacatcccctctatgtacaaccaacacaaaagaagggtccaacatctgtttaacaagaactgacagaggatggtactgtgacaatgcaggatcagtatctttcttcccacaagctgaaacatgtaaagttcaatcaaatcgagtattttgtgacacaatgaacagtttaacattaccaagtgaagtaaatctctgcaatgttgacatattcaaccccaaatatgattgtaaaattatgacttcaaaaacagatgtaagcagctccgttatcacatctctaggagccattgtgtcatgctatggcaaaactaaatgtacagcatccaataaaaatcgtggaatcataaagacattttctaacgggtgcgattatgtatcaaataaaggggtggacactgtgtctgtaggtaacacattatattatgtaaataagcaagaaggtaaaagtctctatgtaaaaggtgaaccaataataaatttctatgacccattagtattcccctctgatgaatttgatgcatcaatatctcaagtcaacgagaagattaaccagagcctagcatttattcgtaaatccgatgaattattacataatgtaaatgctggtaaatccaccacaaatatcatgataactactataattatagtgattatagtaatattgttatcattaattgctgttggactgctcttatactgtaaggccagaagcacaccagtcacactaagcaaagatcaactgagtggtataaataatattgcatttagtaactaaataaaaatagcacctaatcatgttcttacaatggtttactatctgctcatagacaacccatctgtcattggattttcttaaaatctgaacttcatcgaaactctcatctataaaccatctcacttacactatttaagtagattcctagtttatagttatataaaacacaattgcatgccagattaacttaccatctgtaaaaatgaaaactggggcaaatatgtcacgaaggaatccttgcaaatttgaaattcgaggtcattgcttaaatggtaagaggtgtcattttagtcataattattttgaatggccaccccatgcactgcttgtaagacaaaactttatgttaaacagaatacttaagtctatggataaaagtatagataccttatcagaaataagtggagctgcagagttggacagaacagaagagtatgctcttggtgtagttggagtgctagagagttatataggatcaataaacaatataactaaacaatcagcatgtgttgccatgagcaaactcctcactgaactcaatagtgatgatatcaaaaagctgagggacaatgaagagctaaattcacccaagataagagtgtacaatactgtcatatcatatattgaaagcaacaggaaaaacaataaacaaactatccatctgttaaaaagattgccagcagacgtattgaagaaaaccatcaaaaacacattggatatccataagagcataaccatcaacaacccaaaagaatcaactgttagtgatacaaatgaccatgccaaaaataatgatactacctgacaaatatccttgtagtataacttccatactaataacaagtagatgtagagttactatgtataatcaaaagaacacactatatttcaatcaaaacaacccaaataaccatatgtactcaccgaatcaaacattcaatgaaatccattggacctctcaagaattgattgacacaattcaaaattttctacaacatctaggtattattgaggatatatatacaatatatatattagtgtcataacactcaattctaacactcaccacatcgttacattattaattcaaacaattcaagttgtgggacaaaatggatcccattattaatggaaattctgctaatgtttatctaaccgatagttatttaaaaggtgttatctctttctcagagtgtaatgctttaggaagttacatattcaatggtccttatctcaaaaatgattataccaacttaattagtagacaaaatccattaatagaacacatgaatctaaagaaactaaatataacacagtccttaatatctaagtatcataaaggtgaaataaaattagaagaacctacttattttcagtcattacttatgacatacaagagtatgacctcgtcagaacagattgctaccactaatttacttaaaaagataataagaagagctatagaaataagtgatgtcaaagtctatgctatattgaataaactagggcttaaagaaaaggacaagattaaatccaacaatggacaagatgaagacaactcagttattacgaccataatcaaagatgatatactttcagctgttaaagataatcaatctcatcttaaagcagacaaaaatcactctacaaaacaaaaagacacaatcaaaacaacactcttgaagaaattgatgtgttcaatgcaacatcctccatcatggttaatacattggtttaacttatacacaaaattaaacaacatattaacacagtatcgatcaaatgaggtaaaaaaccatgggtttacattgatagataatcaaactcttagtggatttcaatttattttgaaccaatatggttgtatagtttatcataaggaactcaaaagaattactgtgacaacctataatcaattcttgacatggaaagatattagccttagtagattaaatgtttgtttaattacatggattagtaactgcttgaacacattaaataaaagcttaggcttaagatgcggattcaataatgttatcttgacacaactattcctttatggagattgtatactaaagctatttcacaatgaggggttctacataataaaagaggtagagggatttattatgtctctaattttaaatataacagaagaagatcaattcagaaaacgattttataatagtatgctcaacaacatcacagatgctgctaataaagctcagaaaaatctgctatcaagagtatgtcatacattattagataagacagtgtccgataatataataaatggcagatggataattctattaagtaagttccttaaattaattaagcttgcaggtgacaataaccttaacaatctgagtgaactatattttttgttcagaatatttggacacccaatggtagatgaaagacaagccatggatgctgttaaaattaattgcaatgagaccaaattttacttgttaagcagtctgagtatgttaagaggtgcctttatatatagaattataaaagggtttgtaaataattacaacagatggcctactttaagaaatgctattgttttacccttaagatggttaacttactataaactaaacacttatccttctttgttggaacttacagaaagagatttgattgtgttatcaggactacgtttctatcgtgagtttcggttgcctaaaaaagtggatcttgaaatgattataaatgataaagctatatcacctcctaaaaatttgatatggactagtttccctagaaattacatgccatcacacatacaaaactatatagaacatgaaaaattaaaattttccgagagtgataaatcaagaagagtattagagtattatttaagagataacaaattcaatgaatgtgatttatacaactgtgtagttaatcaaagttatctcaacaaccctaatcatgtggtatcattgacaggcaaagaaagagaactcagtgtaggtagaatgtttgcaatgcaaccgggaatgttcagacaggttcaaatattggcagagaaaatgatagctgaaaacattttacaattctttcctgaaagtcttacaagatatggtgatctagaactacaaaaaatattagaactgaaagcaggaataagtaacaaatcaaatcgctacaatgataattacaacaattacattagtaagtgctctatcatcacagatctcagcaaattcaatcaagcatttcgatatgaaacgtcatgtatttgtagtgatgtgctggatgaactgcatggtgtacaatctctattttcctggttacatttaactattcctcatgtcacaataatatgcacatataggcatgcacccccctatataggagatcatattgtagatcttaacaatgtagatgaacaaagtggattatatagatatcacatgggtggcatcgaagggtggtgtcaaaaactatggaccatagaagctatatcactattggatctaatatctctcaaagggaaattctcaattactgctttaattaatggtgacaatcaatcaatagatataagcaaaccaatcagactcatggaaggtcaaactcatgctcaagcagattatttgctagcattaaatagccttaaattactgtataaagagtatgcaggcataggccacaaattaaaaggaactgagacttatatatcacgagatatgcaatttatgagtaaaacaattcaacataacggtgtatattacccagctagtataaagaaagtcctaagagtgggaccgtggataaacactatacttgatgatttcaaagtgagtctagaatctataggtagtttgacacaagaattagaatatagaggtgaaagtctattatgcagtttaatatttagaaatgtatggttatataatcagattgctctacaattaaaaaatcatgcattatgtaacaataaactatatttggacatattaaaggttctgaaacacttaaaaaccttttttaatcttgataatattgatacagcattaacattgtatatgaatttacccatgttatttggtggtggtgatcccaacttgttatatcgaagtttctatagaagaactcctgacttcctcacagaggctatagttcactctgtgttcatacttagttattatacaaaccatgacttaaaagataaacttcaagatctgtcagatgatagattgaataagttcttaacatgcataatcacgtttgacaaaaaccctaatgctgaattcgtaacattgatgagagatcctcaagctttagggtctgagagacaagctaaaattactagcgaaatcaatagactggcagttacagaggttttgagtacagctccaaacaaaatattctccaaaagtgcacaacattatactactacagagatagatctaaatgatattatgcaaaatatagaacctacatatcctcatgggctaagagttgtttatgaaagtttacccttttataaagcagagaaaatagtaaatcttatatcaggtacaaaatctataactaacatactggaaaaaacttctgccatagacttaacagatattgatagagccactgagatgatgaggaaaaacataactttgcttataaggatacttccattggattgtaacagagataaaagagagatattgagtatggaaaacctaagtattactgaattaagcaaatatgttagggaaagatcttggtctttatccaatatagttggtgttacatcacccagtatcatgtatacaatggacatcaaatatactacaagcactatatctagtggcataattatagagaaatataatgttaacagtttaacacgtggtgagagaggacccactaaaccatgggttggttcatctacacaagagaaaaaaacaatgccagtttataatagacaagtcttaaccaaaaaacagagagatcaaatagatctattagcaaaattggattgggtgtatgcatctatagataacaaggatgaattcatggaagaactcagcataggaacccttgggttaacatatgaaaaggccaagaaattatttccacaatatttaagtgtcaattatttgcatcgccttacagtcagtagtagaccatgtgaattccctgcatcaataccagcttatagaacaacaaattatcactttgacactagccctattaatcgcatattaacagaaaagtatggtgatgaagatattgacatagtattccaaaactgtataagctttggccttagtttaatgtcagtagtagaacaatttactaatgtatgtcctaacagaattattctcatacctaagcttaatgagatacatttgatgaaacctcccatattcacaggtgatgttgatattcacaagttaaaacaagtgatacaaaaacagcatatgtttttaccagacaaaataagtttgactcaatatgtggaattattcttaagtaataaaacactcaaatctggatctcatgttaattctaatttaatattggcacataaaatatctgactattttcataatacttacattttaagtactaatttagctggacattggattctgattatacaacttatgaaagattctaaaggtatttttgaaaaagattggggagagggatatataactgatcatatgtttattaatttgaaagttttcttcaatgcttataagacctatctcttgtgttttcataaaggttatggcaaagcaaagctggagtgtgatatgaacacttcagatcttctatgtgtattggaattaatagacagtagttattggaagtctatgtctaaggtatttttagaacaaaaagttatcaaatacattcttagccaagatgcaagtttacatagagtaaaaggatgtcatagcttcaaattatggtttcttaaacgtcttaatgtagcagaattcacagtttgcccttgggttgttaacatagattatcatccaacacatatgaaagcaatattaacttatatagatcttgttagaatgggattgataaatatagatagaatacacattaaaaataaacacaaattcaatgatgaattttatacttctaatctcttctacattaattataacttctcagataatactcatctattaactaaacatataaggattgctaattctgaattagaaaataattacaacaaattatatcatcctacaccagaaaccctagagaatatactagccaatccgattaaaagtaatgacaaaaagacactgaatgactattgtataggtaaaaatgttgactcaataatgttaccattgttatctaataagaagcttattaaatcgtctgcaatgattagaaccaattacagcaaacaagatttgtataatttattccctatggttgtgattgatagaattatagatcattcaggcaatacagccaaatccaaccaactttacactactacttcccaccaaatatccttagtgcacaatagcacatcactttactgcatgcttccttggcatcatattaatagattcaattttgtatttagttctacaggttgtaaaattagtatagagtatattttaaaagatcttaaaattaaagatcccaattgtatagcattcataggtgaaggagcagggaatttattattgcgtacagtagtggaacttcatcctgacataagatatatttacagaagtctgaaagattgcaatgatcatagtttacctattgagtttttaaggctgtacaatggacatatcaacattgattatggtgaaaatttgaccattcctgctacagatgcaaccaacaacattcattggtcttatttacatataaagtttgctgaacctatcagtctttttgtctgtgatgccgaattgtctgtaacagtcaactggagtaaaattataatagaatggagcaagcatgtaagaaagtgcaagtactgttcctcagttaataaatgtatgttaatagtaaaatatcatgctcaagatgatattgatttcaaattagacaatataactatattaaaaacttatgtatgcttaggcagtaagttaaagggatcggaggtttacttagtccttacaataggtcctgcgaatatattcccagtatttaatgtagtacaaaatgctaaattgatactatcaagaaccaaaaatttcatcatgcctaagaaagctgataaagagtctattgatgcaaatattaaaagtttgataccctttctttgttaccctataacaaaaaaaggaattaatactgcattgtcaaaactaaagagtgttgttagtggagatatactatcatattctatagctggacgtaatgaagttttcagcaataaacttataaatcataagcatatgaacatcttaaaatggttcaatcatgttttaaatttcagatcaacagaactaaactataaccatttatatatggtagaatctacatatccttacctaagtgaattgttaaacagcttgacaaccaatgaacttaaaaaactgattaaaatcacaggtagtctgttatacaactttcataatgaataatgaataaagatcttataataaaaattcccatagctatacactaacactgtattcaattatagttattaaaaattaaaaatcatataattttttaaataacttttagtgaactaatcctaaagttatcattttaatcttggaggaataaatttaaaccctaatctaattggtttatatgtgtattaactaaattacgagatattagtttttgacactttttttctcgt

SEQ ID NO: 3 - DNA, 15111 bp, Human respiratory syncytial virus
  source 1..15111: mol_type = other DNA
acgggaaaaaatgcgtacaacaaacttgcataaaccaaaaaaatggggcaaataagaatttgataagtaccacttaaatttaactcccttggttagagatgggcagcaattcattgagtatgataaaagttagattacaaaatttgtttgacaatgatgaagtagcattgttaaaaataacatgctatactgataaattaatacatttaactaatgctttggctaaggcagtgatacatacaatcaaattgaatggcattgtgtttgtgcatgttattacaagtagtgatatttgccctaataataatattgtagtaaaatccaatttcacaacaatgccagtactacaaaatggaggttatatatgggaaatgatggaattaacacattgctctcaacctaatggtctactagatgacaattgtgaaattaaattctccaaaaaactaagtgattcaacaatgaccaattatatgaatcaattatctgaattacttggatttgatcttaatccataaattataattaatatcaactagcaaatcaatgtcactaacaccattagttaatataaaacttaacagaagacaaaaatggggcaaataaatcaattcagccaacccaaccatggacacaacccacaatgataatacaccacaaagactgatgatcacagacatgagaccgttgtcacttgagaccataataacatcactaaccagagacatcataacacacaaatttatatacttgataaatcatgaatgcatagtgagaaaacttgatgaaagacaggccacatttacattcctggtcaactatgaaatgaaactattacacaaagtaggaagcactaaatataaaaaatatactgaatacaacacaaaatatggcactttccctatgccaatattcatcaatcatgatgggttcttagaatgcattggcattaagcctacaaagcatactcccataatatacaagtatgatctcaatccataaatttcaacacaatattcacacaatctaaaacaacaactctatgcataactatactccatagtccagatggagcctgaaaattatagtaatttaaaacttaaggagagatataagatagaagatggggcaaatacaaccatggctcttagcaaagtcaagttgaatgatacactcaacaaagatcaacttctgtcatccagcaaatacaccatccaacggagcacaggagatagtattgatactcctaattatgatgtgcagaaacacatcaataagttatgtggcatgttattaatcacagaagatgctaatcataaattcactgggttaataggtatgttatatgcgatgtctaggttaggaagagaagacaccataaaaatactcagagatgcgggatatcatgtaaaagcaaatggagtagatgtaacaacacatcgtcaagacattaatggaaaagaaatgaaatttgaagtgttaacattggcaagcttaacaactgaaattcaaatcaacattgagatagaatctagaaaatcctacaaaaaaatgctaaaagaaatgggagaggtagctccagaatacaggcatgactctcctgattgtgggatgataatattatgtatagcagcattagtaataactaaattagcagcaggggacagatctggtcttacagccgtgattaggagagctaataatgtcctaaaaaatgaaatgaaacgttacaaaggcttactacccaaggacatagccaacagcttctatgaagtgtttgaaaaacatccccactttatagatgtttttgttcattttggtatagcacaatcttctaccagaggtggcagtagagttgaagggatttttgcaggattgtttatgaatgcctatggtgcagggcaagtgatgttacggtggggagtcttagcaaaatcggttaaaaatattatgttaggacatgctagtgtgcaagcagaaatggaacaagttgttgaggtttatgaatatgcccaaaaattgggtggtgaagcaggattctaccatatattgaacaacccaaaagcatcattattatctttgactcaatttcctcacttctccagtgtagtattaggcaatgctgctggcctaggcataatgggagagtacagaggtacaccgaggaatcaagatctatatgatgcagcaaaggcatatgctgaacaactcaaagaaaatggtgtgattaactacagtgtactagacttgacagcagaagaactagaggctatcaaacatcagcttaatccaaaagataatgatgtagagctttgagttaataaaaaatggggcaaataaatcatcatggaaaagtttgctcctgaattccatggagaagatgcaaacaacagggctactaaattcctagaatcaataaagggcaaattcacatcacccaaagatcccaagaaaaaagatagtatcatatctgtcaactcaatagatatagaagtaaccaaagaaagccctataacatcaaattcaactattatcaacccaacaaatgagacagatgatactgcagggaacaagcccaattatcaaagaaaacctctagtaagtttcaaagaagaccctacaccaagtgataatcccttttctaaactatacaaagaaaccatagaaacatttgataacaatgaagaagaatccagctattcatacgaagaaataaatgatcagacaaacgataatataacagcaagattagataggattgatgaaaaattaagtgaaatactaggaatgcttcacacattagtagtggcaagtgcaggacctacatctgctcgggatggtataagagatgccatggttggtttaagagaagaaatgatagaaaaaatcagaactgaagcattaatgaccaatgacagattagaagctatggcaagactcaggaatgaggaaagtgaaaagatggcaaaagacacatcagatgaagtgtctctcaatccaacatcagagaaattgaacaacctattggaagggaatgatagtgacaatgatctatcacttgaagatttctgattagttaccaatcttcacatcaacacacaataccaacagaagaccaacaaactaaccaacccaatcatccaaccaaacatccatccgccaatcagccaaacagccaacaaaacaaccagccaatccaaaactaaccacccggaaaaaatctataatatagttacaaaaaaaggaaagggtggggcaaatatggaaacatacgtgaacaagcttcacgaaggctccacatacacagctgctgttcaatacaatgtcttagaaaaagacgatgaccctgcatcacttacaatatgggtgcccatgttccaatcatctatgccagcagatttacttataaaagaactagctaatgtcaacatactagtgaaacaaatatccacacccaagggaccttcactaagagtcatgataaactcaagaagtgcagtgctagcacaaatgcccagcaaatttaccatatgcgctaatgtgtccttggatgaaagaagcaaactagcatatgatgtaaccacaccctgtgaaatcaaggcatgtagtctaacatgcctaaaatcaaaaaatatgttgactacagttaaagatctcactatgaagacactcaaccctacacatgatattattgctttatgtgaatttgaaaacatagtaacatcaaaaaaagtcataataccaacatacctaagatccatcagtgtcagaaataaagatctgaacacacttgaaaatataacaaccactgaattcaaaaatgctatcacaaatgcaaaaatcatcccttactcaggattactattagtcatcacagtgactgacaacaaaggagcattcaaatacataaagccacaaagtcaattcatagtagatcttggagcttacctagaaaaagaaagtatatattatgttaccacaaattggaagcacacagctacacgatttgcaatcaaacccatggaagattaacctttttcctctacatcagtgtgttaattcatacaaactttctacctacattcttcacttcaccatcacaatcacaaacactctgtggttcaaccaatcaaacaaaacttatctgaagtcccagatcatcccaagtcattgtttatcagatctagtactcaaataagttaataaaaaatatacacatggggcaaataatcattggaggaaatccaactaatcacaatatctgttaacatagacaagtccacacaccatacagaatcaaccaatggaaaatacatccataacaatagaattctcaagcaaattctggccttactttacactaatacacatgatcacaacaataatctctttgctaatcataatctccatcatgattgcaatactaaacaaactttgtgaatataacgtattccataacaaaacctttgagttaccaagagctcgagttaatacttgataaagtagttaattaaaaatagtcataacaatgaactaggatatcaagactaacaataacattggggcaaatgcaaacatgtccaaaaacaaggaccaacgcaccgctaaaacactcgaaaggacatgggatacccttaatcacctattattcataagctcatgcttatataaattgaaccttaaatccgtcgcacagataaccctatcaatactcgcaatgataatctcaacaagcttaatcatagccgcaataatctttatcgctagcgctaaccataaggtaaccccaacaaccgcaattatacaggacgcaacatcccaaatcaaaaacacaaccccaacatacttaacccaaaacccacaactcggaatctcaccctctaacccatccgaaattacctcacagattacaacgatactcgcaagtacaacccccggagtcaaatcgacactccaatcgacaaccgtaaagactaagaatacaacaacaacccaaacccaacctagtaagcctacaactaagcaacgccaaaacaaacctccctctaaaccgaataacgattttcacttcgaagtgttcaatttcgtaccatgctcaatttgctctaataacccaacatgctgggccatatgcaaacgcatcccaaacaagaaacccggaaagaaaacaaccactaagccaacaaagaaaccaacccttaagacaaccaagaaagatccaaaaccccaaacaactaagtctaaagaggtcccaacaactaagccaaccgaagagccaacaatcaatacaactaagactaatataatcacaaccttactgacatctaacacaaccggaaatcccgaactgacatcccaaatggaaacctttcactcaacctctagcgaaggcaatccctcaccatcccaagtctcaaccactagcgaatacccatcccaacctagctcacctcccaatacccctagacagtagttacttaaaaacatattatcacaaaaggccttgaccaacttaaacagaatcaaaataaactctggggcaaataacaatggagttgctaatcctcaaagcaaatgcaatcacaacaatactaacagccgttacattttgtttcgctagcggacaaaacataaccgaagagttttatcaatctacatgttccgccgtaagtaaggggtatctatccgcacttagaaccggatggtatactagcgtaataacaatcgaactatctaatataaagaagaataagtgtaacggtacagacgctaaggttaaattgattaaacaggaactcgataagtataaaaacgccgtaaccgaattgcaattgttaatgcaatctacacaagctactaataatagggctagacgtgaattgcctagatttatgaattatacacttaataacgctaagaaaactaacgttacactatctaagaaacgaaaacgtagattcttagggtttttactcggagtcggttccgcaatcgctagcggagtcgccgtaagtaaagtgttacacctcgaaggcgaagtgaataagataaaatccgcactattatcaactaataaggcagtcgttagcctatctaacggagtcagcgtattgacatctaaagtgttagacttaaagaattatatagataagcaattgttaccaatcgttaataaacaatcatgttcaatatccaatatcgaaaccgtaatcgaatttcaacagaagaataatagattactcgaaattactagagaatttagcgtaaacgctggcgtaacaacacccgtaagtacatatatgttaactaattccgaactgttaagcttaattaacgatatgccaattactaacgatcagaagaaattgatgtctaataacgtacaaatcgttagacagcaatcatattcaattatgtcaattataaaagaagaggtactcgcatacgtagtgcaattacccctatatggcgtaatagatacaccatgttggaaattgcatacaagtccactatgtacaactaatacaaaagagggatctaatatatgcttaactagaaccgatagggggtggtattgcgataacgcaggtagcgtaagtttctttccacaagccgaaacatgtaaagtgcaatctaatagagtgttttgcgatacaatgaatagcttaacactacctagcgaagtcaatctatgtaacgtcgatatattcaatcctaaatatgattgcaaaattatgactagtaagactgacgtaagtagtagcgtaattactagtctcggtgcaatagtgtcatgttatggtaagactaagtgtaccgctagcaataagaatagggggataataaaaacatttagtaacggttgcgattacgttagtaataagggagtcgataccgtaagcgtaggtaatacactatattatgttaataaacaggaaggtaagtcattatacgttaaaggcgaacctataattaatttttacgatccattagtgtttccatccgacgaattcgacgctagtataagtcaggtaaacgaaaagattaaccaatcactcgcattcatacgaaaatccgacgaactgttacacaacgttaacgcaggtaagagtacaactaacataatgataacaacaattataatcgttataatcgttatactgttaagcttaatcgcagtcggattactgttatattgtaaagctagatcaacacccgtaacactatctaaagaccaattatccggtataaataatatcgcattctcaaactaaataaaaatagcacctaatcatgttcttacaatggtttactatctgctcatagacaacccatctgtcattggattttcttaaaatctgaacttcatcgaaactctcatctataaaccatctcacttacactatttaagtagattcctagtttatagttatataaaacacaattgcatgccagattaacttaccatctgtaaaaatgaaaactggggcaaatatgtcacgaaggaatccttgcaaatttgaaattcgaggtcattgcttaaatggtaagaggtgtcattttagtcataattattttgaatggccaccccatgcactgcttgtaagacaaaactttatgttaaacagaatacttaagtctatggataaaagtatagataccttatcagaaataagtggagctgcagagttggacagaacagaagagtatgctcttggtgtagttggagtgctagagagttatataggatcaataaacaatataactaaacaatcagcatgtgttgccatgagcaaactcctcactgaactcaatagtgatgatatcaaaaagctgagggacaatgaagagctaaattcacccaagataagagtgtacaatactgtcatatcatatattgaaagcaacaggaaaaacaataaacaaactatccatctgttaaaaagattgccagcagacgtattgaagaaaaccatcaaaaacacattggatatccataagagcataaccatcaacaacccaaaagaatcaactgttagtgatacaaatgaccatgccaaaaataatgatactacctgacaaatatccttgtagtataacttccatactaataacaagtagatgtagagttactatgtataatcaaaagaacacactatatttcaatcaaaacaacccaaataaccatatgtactcaccgaatcaaacattcaatgaaatccattggacctctcaagaattgattgacacaattcaaaattttctacaacatctaggtattattgaggatatatatacaatatatatattagtgtcataacactcaattctaacactcaccacatcgttacattattaattcaaacaattcaagttgtgggacaaaatggatcccattattaatggaaattctgctaatgtttatctaaccgatagttatttaaaaggtgttatctctttctcagagtgtaatgctttaggaagttacatattcaatggtccttatctcaaaaatgattataccaacttaattagtagacaaaatccattaatagaacacatgaatctaaagaaactaaatataacacagtccttaatatctaagtatcataaaggtgaaataaaattagaagaacctacttattttcagtcattacttatgacatacaagagtatgacctcgtcagaacagattgctaccactaatttacttaaaaagataataagaagagctatagaaataagtgatgtcaaagtctatgctatattgaataaactagggcttaaagaaaaggacaagattaaatccaacaatggacaagatgaagacaactcagttattacgaccataatcaaagatgatatactttcagctgttaaagataatcaatctcatcttaaagcagacaaaaatcactctacaaaacaaaaagacacaatcaaaacaacactcttgaagaaattgatgtgttcaatgcaacatcctccatcatggttaatacattggtttaacttatacacaaaattaaacaacatattaacacagtatcgatcaaatgaggtaaaaaaccatgggtttacattgatagataatcaaactcttagtggatttcaatttattttgaaccaatatggttgtatagtttatcataaggaactcaaaagaattactgtgacaacctataatcaattcttgacatggaaagatattagccttagtagattaaatgtttgtttaattacatggattagtaactgcttgaacacattaaataaaagcttaggcttaagatgcggattcaataatgttatcttgacacaactattcctttatggagattgtatactaaagctatttcacaatgaggggttctacataataaaagaggtagagggatttattatgtctctaattttaaatataacagaagaagatcaattcagaaaacgattttataatagtatgctcaacaacatcacagatgctgctaataaagctcagaaaaatctgctatcaagagtatgtcatacattattagataagacagtgtccgataatataataaatggcagatggataattctattaagtaagttccttaaattaattaagcttgcaggtgacaataaccttaacaatctgagtgaactatattttttgttcagaatatttggacacccaatggtagatgaaagacaagccatggatgctgttaaaattaattgcaatgagaccaaattttacttgttaagcagtctgagtatgttaagaggtgcctttatatatagaattataaaagggtttgtaaataattacaacagatggcctactttaagaaatgctattgttttacccttaagatggttaacttactataaactaaacacttatccttctttgttggaacttacagaaagagatttgattgtgttatcaggactacgtttctatcgtgagtttcggttgcctaaaaaagtggatcttgaaatgattataaatgataaagctatatcacctcctaaaaatttgatatggactagtttccctagaaattacatgccatcacacatacaaaactatatagaacatgaaaaattaaaattttccgagagtgataaatcaagaagagtattagagtattatttaagagataacaaattcaatgaatgtgatttatacaactgtgtagttaatcaaagttatctcaacaaccctaatcatgtggtatcattgacaggcaaagaaagagaactcagtgtaggtagaatgtttgcaatgcaaccgggaatgttcagacaggttcaaatattggcagagaaaatgatagctgaaaacattttacaattctttcctgaaagtcttacaagatatggtgatctagaactacaaaaaatattagaactgaaagcaggaataagtaacaaatcaaatcgctacaatgataattacaacaattacattagtaagtgctctatcatcacagatctcagcaaattcaatcaagcatttcgatatgaaacgtcatgtatttgtagtgatgtgctggatgaactgcatggtgtacaatctctattttcctggttacatttaactattcctcatgtcacaataatatgcacatataggcatgcacccccctatataggagatcatattgtagatcttaacaatgtagatgaacaaagtggattatatagatatcacatgggtggcatcgaagggtggtgtcaaaaactatggaccatagaagctatatcactattggatctaatatctctcaaagggaaattctcaattactgctttaattaatggtgacaatcaatcaatagatataagcaaaccaatcagactcatggaaggtcaaactcatgctcaagcagattatttgctagcattaaatagccttaaattactgtataaagagtatgcaggcataggccacaaattaaaaggaactgagacttatatatcacgagatatgcaatttatgagtaaaacaattcaacataacggtgtatattacccagctagtataaagaaagtcctaagagtgggaccgtggataaacactatacttgatgatttcaaagtgagtctagaatctataggtagtttgacacaagaattagaatatagaggtgaaagtctattatgcagtttaatatttagaaatgtatggttatataatcagattgctctacaattaaaaaatcatgcattatgtaacaataaactatatttggacatattaaaggttctgaaacacttaaaaaccttttttaatcttgataatattgatacagcattaacattgtatatgaatttacccatgttatttggtggtggtgatcccaacttgttatatcgaagtttctatagaagaactcctgacttcctcacagaggctatagttcactctgtgttcatacttagttattatacaaaccatgacttaaaagataaacttcaagatctgtcagatgatagattgaataagttcttaacatgcataatcacgtttgacaaaaaccctaatgctgaattcgtaacattgatgagagatcctcaagctttagggtctgagagacaagctaaaattactagcgaaatcaatagactggcagttacagaggttttgagtacagctccaaacaaaatattctccaaaagtgcacaacattatactactacagagatagatctaaatgatattatgcaaaatatagaacctacatatcctcatgggctaagagttgtttatgaaagtttacccttttataaagcagagaaaatagtaaatcttatatcaggtacaaaatctataactaacatactggaaaaaacttctgccatagacttaacagatattgatagagccactgagatgatgaggaaaaacataactttgcttataaggatacttccattggattgtaacagagataaaagagagatattgagtatggaaaacctaagtattactgaattaagcaaatatgttagggaaagatcttggtctttatccaatatagttggtgttacatcacccagtatcatgtatacaatggacatcaaatatactacaagcactatatctagtggcataattatagagaaatataatgttaacagtttaacacgtggtgagagaggacccactaaaccatgggttggttcatctacacaagagaaaaaaacaatgccagtttataatagacaagtcttaaccaaaaaacagagagatcaaatagatctattagcaaaattggattgggtgtatgcatctatagataacaaggatgaattcatggaagaactcagcataggaacccttgggttaacatatgaaaaggccaagaaattatttccacaatatttaagtgtcaattatttgcatcgccttacagtcagtagtagaccatgtgaattccctgcatcaataccagcttatagaacaacaaattatcactttgacactagccctattaatcgcatattaacagaaaagtatggtgatgaagatattgacatagtattccaaaactgtataagctttggccttagtttaatgtcagtagtagaacaatttactaatgtatgtcctaacagaattattctcatacctaagcttaatgagatacatttgatgaaacctcccatattcacaggtgatgttgatattcacaagttaaaacaagtgatacaaaaacagcatatgtttttaccagacaaaataagtttgactcaatatgtggaattattcttaagtaataaaacactcaaatctggatctcatgttaattctaatttaatattggcacataaaatatctgactattttcataatacttacattttaagtactaatttagctggacattggattctgattatacaacttatgaaagattctaaaggtatttttgaaaaagattggggagagggatatataactgatcatatgtttattaatttgaaagttttcttcaatgcttataagacctatctcttgtgttttcataaaggttatggcaaagcaaagctggagtgtgatatgaacacttcagatcttctatgtgtattggaattaatagacagtagttattggaagtctatgtctaaggtatttttagaacaaaaagttatcaaatacattcttagccaagatgcaagtttacatagagtaaaaggatgtcatagcttcaaattatggtttcttaaacgtcttaatgtagcagaattcacagtttgcccttgggttgttaacatagattatcatccaacacatatgaaagcaatattaacttatatagatcttgttagaatgggattgataaatatagatagaatacacattaaaaataaacacaaattcaatgatgaattttatacttctaatctcttctacattaattataacttctcagataatactcatctattaactaaacatataaggattgctaattctgaattagaaaataattacaacaaattatatcatcctacaccagaaaccctagagaatatactagccaatccgattaaaagtaatgacaaaaagacactgaatgactattgtataggtaaaaatgttgactcaataatgttaccattgttatctaataagaagcttattaaatcgtctgcaatgattagaaccaattacagcaaacaagatttgtataatttattccctatggttgtgattgatagaattatagatcattcaggcaatacagccaaatccaaccaactttacactactacttcccaccaaatatccttagtgcacaatagcacatcactttactgcatgcttccttggcatcatattaatagattcaattttgtatttagttctacaggttgtaaaattagtatagagtatattttaaaagatcttaaaattaaagatcccaattgtatagcattcataggtgaaggagcagggaatttattattgcgtacagtagtggaacttcatcctgacataagatatatttacagaagtctgaaagattgcaatgatcatagtttacctattgagtttttaaggctgtacaatggacatatcaacattgattatggtgaaaatttgaccattcctgctacagatgcaaccaacaacattcattggtcttatttacatataaagtttgctgaacctatcagtctttttgtctgtgatgccgaattgtctgtaacagtcaactggagtaaaattataatagaatggagcaagcatgtaagaaagtgcaagtactgttcctcagttaataaatgtatgttaatagtaaaatatcatgctcaagatgatattgatttcaaattagacaatataactatattaaaaacttatgtatgcttaggcagtaagttaaagggatcggaggtttacttagtccttacaataggtcctgcgaatatattcccagtatttaatgtagtacaaaatgctaaattgatactatcaagaaccaaaaatttcatcatgcctaagaaagctgataaagagtctattgatgcaaatattaaaagtttgataccctttctttgttaccctataacaaaaaaaggaattaatactgcattgtcaaaactaaagagtgttgttagtggagatatactatcatattctatagctggacgtaatgaagttttcagcaataaacttataaatcataagcatatgaacatcttaaaatggttcaatcatgttttaaatttcagatcaacagaactaaactataaccatttatatatggtagaatctacatatccttacctaagtgaattgttaaacagcttgacaaccaatgaacttaaaaaactgattaaaatcacaggtagtctgttatacaactttcataatgaataatgaataaagatcttataataaaaattcccatagctatacactaacactgtattcaattatagttattaaaaattaaaaatcatataattttttaaataacttttagtgaactaatcctaaagttatcattttaatcttggaggaataaatttaaaccctaatctaattggtttatatgtgtattaactaaattacgagatattagtttttgacactttttttctcgt

SEQ ID NO: 4 - DNA, 15111 bp, Human respiratory syncytial virus
  source 1..15111: mol_type = other DNA
acgggaaaaaatgcgtacaacaaacttgcataaaccaaaaaaatggggcaaataagaatttgataagtaccacttaaatttaactcccttggttagagatgggcagcaattcattgagtatgataaaagttagattacaaaatttgtttgacaatgatgaagtagcattgttaaaaataacatgctatactgataaattaatacatttaactaatgctttggctaaggcagtgatacatacaatcaaattgaatggcattgtgtttgtgcatgttattacaagtagtgatatttgccctaataataatattgtagtaaaatccaatttcacaacaatgccagtactacaaaatggaggttatatatgggaaatgatggaattaacacattgctctcaacctaatggtctactagatgacaattgtgaaattaaattctccaaaaaactaagtgattcaacaatgaccaattatatgaatcaattatctgaattacttggatttgatcttaatccataaattataattaatatcaactagcaaatcaatgtcactaacaccattagttaatataaaacttaacagaagacaaaaatggggcaaataaatcaattcagccaacccaaccatggacacaacccacaatgataatacaccacaaagactgatgatcacagacatgagaccgttgtcacttgagaccataataacatcactaaccagagacatcataacacacaaatttatatacttgataaatcatgaatgcatagtgagaaaacttgatgaaagacaggccacatttacattcctggtcaactatgaaatgaaactattacacaaagtaggaagcactaaatataaaaaatatactgaatacaacacaaaatatggcactttccctatgccaatattcatcaatcatgatgggttcttagaatgcattggcattaagcctacaaagcatactcccataatatacaagtatgatctcaatccataaatttcaacacaatattcacacaatctaaaacaacaactctatgcataactatactccatagtccagatggagcctgaaaattatagtaatttaaaacttaaggagagatataagatagaagatggggcaaatacaaccatggctcttagcaaagtcaagttgaatgatacactcaacaaagatcaacttctgtcatccagcaaatacaccatccaacggagcacaggagatagtattgatactcctaattatgatgtgcagaaacacatcaataagttatgtggcatgttattaatcacagaagatgctaatcataaattcactgggttaataggtatgttatatgcgatgtctaggttaggaagagaagacaccataaaaatactcagagatgcgggatatcatgtaaaagcaaatggagtagatgtaacaacacatcgtcaagacattaatggaaaagaaatgaaatttgaagtgttaacattggcaagcttaacaactgaaattcaaatcaacattgagatagaatctagaaaatcctacaaaaaaatgctaaaagaaatgggagaggtagctccagaatacaggcatgactctcctgattgtgggatgataatattatgtatagcagcattagtaataactaaattagcagcaggggacagatctggtcttacagccgtgattaggagagctaataatgtcctaaaaaatgaaatgaaacgttacaaaggcttactacccaaggacatagccaacagcttctatgaagtgtttgaaaaacatccccactttatagatgtttttgttcattttggtatagcacaatcttctaccagaggtggcagtagagttgaagggatttttgcaggattgtttatgaatgcctatggtgcagggcaagtgatgttacggtggggagtcttagcaaaatcggttaaaaatattatgttaggacatgctagtgtgcaagcagaaatggaacaagttgttgaggtttatgaatatgcccaaaaattgggtggtgaagcaggattctaccatatattgaacaacccaaaagcatcattattatctttgactcaatttcctcacttctccagtgtagtattaggcaatgctgctggcctaggcataatgggagagtacagaggtacaccgaggaatcaagatctatatgatgcagcaaaggcatatgctgaacaactcaaagaaaatggtgtgattaactacagtgtactagacttgacagcagaagaactagaggctatcaaacatcagcttaatccaaaagataatgatgtagagctttgagttaataaaaaatggggcaaataaatcatcatggaaaagtttgctcctgaattccatggagaagatgcaaacaacagggctactaaattcctagaatcaataaagggcaaattcacatcacccaaagatcccaagaaaaaagatagtatcatatctgtcaactcaatagatatagaagtaaccaaagaaagccctataacatcaaattcaactattatcaacccaacaaatgagacagatgatactgcagggaacaagcccaattatcaaagaaaacctctagtaagtttcaaagaagaccctacaccaagtgataatcccttttctaaactatacaaagaaaccatagaaacatttgataacaatgaagaagaatccagctattcatacgaagaaataaatgatcagacaaacgataatataacagcaagattagataggattgatgaaaaattaagtgaaatactaggaatgcttcacacattagtagtggcaagtgcaggacctacatctgctcgggatggtataagagatgccatggttggtttaagagaagaaatgatagaaaaaatcagaactgaagcattaatgaccaatgacagattagaagctatggcaagactcaggaatgaggaaagtgaaaagatggcaaaagacacatcagatgaagtgtctctcaatccaacatcagagaaattgaacaacctattggaagggaatgatagtgacaatgatctatcacttgaagatttctgattagttaccaatcttcacatcaacacacaataccaacagaagaccaacaaactaaccaacccaatcatccaaccaaacatccatccgccaatcagccaaacagccaacaaaacaaccagccaatccaaaactaaccacccggaaaaaatctataatatagttacaaaaaaaggaaagggtggggcaaatatggaaacatacgtgaacaagcttcacgaaggctccacatacacagctgctgttcaatacaatgtcttagaaaaagacgatgaccctgcatcacttacaatatgggtgcccatgttccaatcatctatgccagcagatttacttataaaagaactagctaatgtcaacatactagtgaaacaaatatccacacccaagggaccttcactaagagtcatgataaactcaagaagtgcagtgctagcacaaatgcccagcaaatttaccatatgcgctaatgtgtccttggatgaaagaagcaaactagcatatgatgtaaccacaccctgtgaaatcaaggcatgtagtctaacatgcctaaaatcaaaaaatatgttgactacagttaaagatctcactatgaagacactcaaccctacacatgatattattgctttatgtgaatttgaaaacatagtaacatcaaaaaaagtcataataccaacatacctaagatccatcagtgtcagaaataaagatctgaacacacttgaaaatataacaaccactgaattcaaaaatgctatcacaaatgcaaaaatcatcccttactcaggattactattagtcatcacagtgactgacaacaaaggagcattcaaatacataaagccacaaagtcaattcatagtagatcttggagcttacctagaaaaagaaagtatatattatgttaccacaaattggaagcacacagctacacgatttgcaatcaaacccatggaagattaacctttttcctctacatcagtgtgttaattcatacaaactttctacctacattcttcacttcaccatcacaatcacaaacactctgtggttcaaccaatcaaacaaaacttatctgaagtcccagatcatcccaagtcattgtttatcagatctagtactcaaataagttaataaaaaatatacacatggggcaaataatcattggaggaaatccaactaatcacaatatctgttaacatagacaagtccacacaccatacagaatcaaccaatggaaaatacatccataacaatagaattctcaagcaaattctggccttactttacactaatacacatgatcacaacaataatctctttgctaatcataatctccatcatgattgcaatactaaacaaactttgtgaatataacgtattccataacaaaacctttgagttaccaagagctcgagttaatacttgataaagtagttaattaaaaatagtcataacaatgaactaggatatcaagactaacaataacattggggcaaatgcaaacatgtccaaaaacaaggaccaacgcaccgctaagacattagaaaggacctgggacactctcaatcatttattattcatatcatcgtgcttatataagttaaatcttaaatctgtagcacaaatcacattatccattctggcaatgataatctcaacttcacttataattgcagccatcatattcatagcctcggcaaaccacaaagtcacaccaacaactgcaatcatacaagatgcaacaagccagatcaagaacacaaccccaacatacctcacccagaatcctcagcttggaatcagtccctctaatccgtctgaaattacatcacaaatcaccaccatactagcttcaacaacaccaggagtcaagtcaaccctgcaatccacaacagtcaagaccaaaaacacaacaacaactcaaacacaacccagcaagcccaccacaaaacaacgccaaaacaaaccaccaagcaaacccaataatgattttcactttgaagtgttcaactttgtaccctgcagcatatgcagcaacaatccaacctgctgggctatctgcaaaagaataccaaacaaaaaaccaggaaagaaaaccactaccaagcccacaaaaaaaccaaccctcaagacaaccaaaaaagatcccaaacctcaaaccactaaatcaaaggaagtacccaccaccaagcccacagaagagccaaccatcaacaccaccaaaacaaacatcataactacactactcacctccaacaccacaggaaatccagaactcacaagtcaaatggaaaccttccactcaacttcctccgaaggcaatccaagcccttctcaagtctctacaacatccgagtacccatcacaaccttcatctccacccaacacaccacgccagtagttacttaaaaacatattatcacaaaaggccttgaccaacttaaacagaatcaaaataaactctggggcaaataacaatggagttgctaatcctcaaagcaaatgcaattaccacaatcctcactgcagtcacattttgttttgcttctggtcaaaacatcactgaagaattttatcaatcaacatgcagtgcagttagcaaaggctatcttagtgctctgagaactggttggtataccagtgttataactatagaattaagtaatatcaagaaaaataagtgtaatggaacagatgctaaggtaaaattgataaaacaagaattagataaatataaaaatgctgtaacagaattgcagttgctcatgcaaagcacacaagcaacaaacaatcgagccagaagagaactaccaaggtttatgaattatacactcaacaatgccaaaaaaaccaatgtaacattaagcaagaaaaggaaaagaagatttcttggttttttgttaggtgttggatctgcaatcgccagtggcgttgctgtatctaaggtcctgcacctagaaggggaagtgaacaagatcaaaagtgctctactatccacaaacaaggctgtagtcagcttatcaaatggagttagtgttttaaccagcaaagtgttagacctcaaaaactatatagataaacaattgttacctattgtgaacaagcaaagctgcagcatatcaaatatagaaactgtgatagagttccaacaaaagaacaacagactactagagattaccagggaatttagtgttaatgcaggcgtaactacacctgtaagcacttacatgttaactaatagtgaattattgtcattaatcaatgatatgcctataacaaatgatcagaaaaagttaatgtccaacaatgttcaaatagttagacagcaaagttactctatcatgtccataataaaagaggaagtcttagcatatgtagtacaattaccactatatggtgttatagatacaccctgttggaaactacacacatcccctctatgtacaaccaacacaaaagaagggtccaacatctgtttaacaagaactgacagaggatggtactgtgacaatgcaggatcagtatctttcttcccacaagctgaaacatgtaaagttcaatcaaatcgagtattttgtgacacaatgaacagtttaacattaccaagtgaagtaaatctctgcaatgttgacatattcaaccccaaatatgattgtaaaattatgacttcaaaaacagatgtaagcagctccgttatcacatctctaggagccattgtgtcatgctatggcaaaactaaatgtacagcatccaataaaaatcgtggaatcataaagacattttctaacgggtgcgattatgtatcaaataaaggggtggacactgtgtctgtaggtaacacattatattatgtaaataagcaagaaggtaaaagtctctatgtaaaaggtgaaccaataataaatttctatgacccattagtattcccctctgatgaatttgatgcatcaatatctcaagtcaacgagaagattaaccagagcctagcatttattcgtaaatccgatgaattattacataatgtaaatgctggtaaatccaccacaaatatcatgataactactataattatagtgattatagtaatattgttatcattaattgctgttggactgctcttatactgtaaggccagaagcacaccagtcacactaagcaaagatcaactgagtggtataaataatattgcatttagtaactaaataaaaatagcacctaatcatgttcttacaatggtttactatctgctcatagacaacccatctgtcattggattttcttaaaatctgaacttcatcgaaactctcatctataaaccatctcacttacactatttaagtagattcctagtttatagttatataaaacacaattgcatgccagattaacttaccatctgtaaaaatgaaaactggggcaaatatgtcacgaaggaatccttgcaaatttgaaattcgaggtcattgcttaaatggtaagaggtgtcattttagtcataattattttgaatggccaccccatgcactgcttgtaagacaaaactttatgttaaacagaatacttaagtctatggataaaagtatagataccttatcagaaataagtggagctgcagagttggacagaacagaagagtatgctcttggtgtagttggagtgctagagagttatataggatcaataaacaatataactaaacaatcagcatgtgttgccatgagcaaactcctcactgaactcaatagtgatgatatcaaaaagctgagggacaatgaagagctaaattcacccaagataagagtgtacaatactgtcatatcatatattgaaagcaacaggaaaaacaataaacaaactatccatctgttaaaaagattgccagcagacgtattgaagaaaaccatcaaaaacacattggatatccataagagcataaccatcaacaacccaaaagaatcaactgttagtgatacaaatgaccatgccaaaaataatgatactacctgacaaatatccttgtagtataacttccatactaataacaagtagatgtagagttactatgtataatcaaaagaacacactatatttcaatcaaaacaacccaaataaccatatgtactcaccgaatcaaacattcaatgaaatccattggacctctcaagaattgattgacacaattcaaaattttctacaacatctaggtattattgaggatatatatacaatatatatattagtgtcataacactcaattctaacactcaccacatcgttacattattaattcaaacaattcaagttgtgggacaaaatggatcccattattaatggaaattctgctaacgtatacttaaccgatagttatttaaaaggcgtaatcagttttagcgaatgtaacgcattagggtcatatatctttaacggtccatatcttaaaaacgattatactaatctaatcagtagacagaatccgttaatcgaacatatgaatcttaagaaactgaatatcacacaatctttgatcagtaagtatcataaaggcgaaatcaaactcgaagaacctacatattttcaatcactattaatgacatataagtctatgacatctagcgaacagatcgctactactaatctgttgaagaaaattattagacgagctatagagatatctgacgttaaggtatacgctatactgaataaattggggttaaaagagaaagataagataaaatctaataacggtcaagacgaagataatagtgtaattactacaattattaaagacgatatactatccgcagtgaaggataatcaatcacatcttaaagccgataaaaatcatagtactaaacaaaaagatacaattaaaactacattgttaaagaaattgatgtgttctatgcaacatccacctagttggttaatacattggtttaacttatacactaagttgaacaatatacttacacaatatcgatcaaacgaagtgaaaaatcacggttttacattgatagataatcaaacattaagcggatttcaattcatacttaaccaatacggatgtatagtgtatcataaagaattgaaacgtataaccgttacaacatataatcaattcttaacatggaaagatataagtctatctagattgaacgtatgcttaattacatggatttcgaattgtcttaatacacttaataaatcattagggttaagatgcggatttaataacgttatacttacacaattgttcttatacggagattgtatacttaagttgttccataacgaagggttttatataataaaagaggttgagggatttataatgtcattgatactgaatattaccgaagaggatcaatttagaaaaagattctataatagtatgttaaacaatataactgacgcagctaataaagcgcagaagaatctgttatctagagtatgtcatacattgttagacaaaacagtgagcgataatattataaacggtagatggattatactgttatctaaattcttaaaattgattaagttggcaggtgacaataaccttaataacttaagcgaattgtatttcttattcagaatattcggacatcctatggttgacgaacgacaagctatggacgcagtgaagattaattgtaacgaaactaaattctatctattatctagtctatctatgcttagaggcgcattcatatatagaattataaaagggttcgttaataattataatagatggcctacacttagaaacgctatagtgttaccacttagatggttaacatattataaattgaatacatatcctagtttactcgaattaaccgaacgcgatctgatagtgttaagcggacttagattctatagagagtttagattgcctaagaaagtcgatctcgaaatgataattaacgataaggcaattagtccacctaaaaacttaatatggacaagcttccctagaaattatatgcctagtcatatacaaaattatatcgaacacgaaaaattgaaatttagcgaatccgataagtctagaagagtgttagagtattacttacgcgataataaatttaacgaatgcgatctatataattgcgtagtgaaccaatcatatcttaataatcctaatcacgtagtgagtcttacaggtaaggaaagagagttgagcgtaggtagaatgttcgctatgcaacccggtatgtttagacaagtgcaaatactcgcagaaaagatgatagccgaaaatatactgcaattctttcccgaatcattgactagatacggagatttagaattgcaaaagatactcgaattgaaagcaggtatatctaataagtctaatagatataacgataattataataattatatatctaagtgtagtattattaccgatctatctaaattcaatcaggcatttagatacgaaactagttgtatatgctcagacgtattagacgaattacacggagtgcaatctttgtttagttggttacatttaactatacctcacgttacaattatatgtacatatagacacgcaccaccatatataggcgatcatatagtcgatctgaataacgtagacgaacaatccggattgtatagatatcacatgggtggcatagagggatggtgtcaaaaattgtggactatagaggcaattagtctgttagatctaattagtcttaagggtaagttttcgattaccgcattgattaacggtgataatcaatcaattgatatatctaaaccgatacggttaatggagggacaaacacacgctcaagccgattacttactcgcacttaattcacttaaactgttatacaaagagtacgcaggtatagggcataaacttaagggtacagagacatatataagtagggatatgcaatttatgagtaagactatacaacataacggagtgtattatcccgctagtataaagaaagtgcttagagtcggaccttggattaatactatattagacgattttaaggttagtctcgaatcaatcggatcattgacacaagagttggagtatagaggcgaatctctattatgctcattgatttttagaaacgtatggttatacaatcagattgcattgcaattgaaaaatcacgcactatgtaataataagttgtacttagacatacttaaagtgttaaaacatcttaaaacattctttaatctcgataatatagataccgcattaacattgtatatgaatctacctatgttattcggagggggagatcctaatctattgtatagatcattctatagacgtacacctgatttcttaaccgaagctatagtgcatagcgtattcatactatcatattatactaatcacgatcttaaagataagttgcaggatctatctgacgatagattgaataaattcttaacatgtattataacattcgataaaaatcctaacgctgaattcgttacacttatgagagatccacaagcattaggttcagagagacaggctaaaattactagcgaaattaatagattagccgttaccgaagtgttaagtaccgcacctaataagatattctctaaatccgctcaacattatacaacaaccgaaatagatcttaacgatattatgcaaaatatcgaacctacatatcctcacggattacgcgtagtttacgaatcattaccattctataaagccgaaaagatcgttaacttaattagcggtacaaaatcaattactaatatactcgaaaagactagcgcaattgatttaaccgatatagatagagctaccgaaatgatgcgtaaaaatataacattactgatacgtatactaccattagattgtaatagggataaaagagagatactatctatggagaatctatcaattacagaattgtcaaaatacgttagggaacgatcatggtcactatctaatatcgtaggcgtaactagtcctagtattatgtatactatggatattaagtatacaactagtacaattagtagcggtataataatcgaaaaatataacgttaatagtctaacacgtggtgaaaggggacctacaaaaccttgggtcggatctagtacacaagagaagaaaactatgcccgtatataatagacaggtattgactaagaaacaacgagatcaaatagatctattagctaaactcgattgggtatacgctagtatagataataaagacgaatttatggaagagttgtcaatcggtacattagggttaacatacgaaaaagctaagaaattgttcccacaatatctatcagtgaattatctacatagattgacagtgagtagtagaccatgcgaatttcccgctagtatacccgcatatagaactactaattatcatttcgatactagtccaattaatagaatattaaccgaaaaatacggagacgaagatatagatatcgtattccaaaattgtattagtttcggattgagtcttatgtccgtagtcgaacaatttactaacgtatgtcctaataggattatactgatacctaaattgaacgaaatacatcttatgaaacctcctatttttacaggcgatgtcgatatacacaaattgaaacaggttatacaaaaacaacatatgttcttacccgataagatatcgttaacgcaatacgttgagttgttcttatcaaataaaacacttaaatcaggtagtcacgttaatagtaatctgatactcgcacataaaattagcgattactttcataatacatatatattgagtactaacttagccggacattggatactgattatacaattgatgaaagatagtaagggtatattcgaaaaagattggggtgagggatatataaccgatcatatgtttataaaccttaaggtcttctttaacgcatataaaacttatctattatgttttcataagggatacggtaaggctaaactcgaatgcgatatgaatacatccgatctattatgcgtactcgaattaattgatagtagctattggaaatctatgagtaaggtattcttagagcaaaaggtgatcaagtatatactatctcaagacgctagtttgcatagggttaagggatgtcatagttttaaattatggtttcttaaaagattgaacgtagccgaatttacagtatgtccttgggtcgttaacatagattatcatcctacacatatgaaagctatacttacatatatagatctagtgagaatgggattgattaacatagatagaatacatataaagaataaacataaatttaacgacgaattctatactagtaatctattctatataaattataatttttccgataatacacatctattaactaaacatatacgtatagctaatagcgaactcgaaaataattataataaattgtatcatcctacacccgaaacattagagaatatactcgctaatccgattaaatctaacgataagaaaacacttaacgattattgtataggtaaaaacgttgattcaattatgttaccattactatcaaataagaaattgattaaatctagcgctatgattagaactaattatagtaaacaggatctatataacttattccctatggtcgtaattgatagaattatagatcattccggtaataccgctaaatctaatcaattgtatacaactactagtcatcaaatatcattagtgcataatagtactagtctatattgtatgttaccatggcatcatattaatagattcaatttcgtttttagtagtacagggtgtaaaattagtatagagtatatacttaaagatcttaaaattaaagatcctaattgtattgcattcataggcgaaggcgcaggtaatctgttacttagaacagtagtcgaattgcatcccgatattagatatatatatagatcacttaaagattgtaacgatcatagtctaccaatcgaattccttagattgtataacggtcatataaacatagattacggcgaaaacttaacgatacccgctactgacgctactaataatatacattggtcatacttacatattaaattcgcagaacctataagtctattcgtatgcgacgcagaattatccgttacagtgaattggtctaaaattattatcgaatggtctaaacacgttagaaaatgcaaatattgttctagcgttaataagtgtatgttaatcgttaagtatcacgctcaagacgatatagattttaaattagataatataactatacttaaaacatacgtatgcttaggtagtaagcttaagggtagcgaagtatacttagtgttaacgataggtccagctaatatttttcccgtttttaacgtagtgcaaaacgctaaattgattctatctagaactaaaaattttataatgcctaagaaagctgataaagagtcaattgacgctaatataaaatcattgataccattcttatgttatcctataactaagaaagggattaataccgcactatctaaacttaaatccgtagtgagcggagatatactatcttatagtatagccggtagaaacgaagtttttagtaataaattgattaatcataaacatatgaatatacttaaatggtttaatcacgtacttaattttagatcaaccgaattgaattataatcatctatatatggtcgaatctacatatccatacttatccgaactgttaaactcattgactactaacgaattgaagaaattgattaaaattacaggtagtctgttatacaattttcataacgaataatgaataaagatcttataataaaaattcccatagctatacactaacactgtattcaattatagttattaaaaattaaaaatcatataattttttaaataacttttagtgaactaatcctaaagttatcattttaatcttggaggaataaatttaaaccctaatctaattggtttatatgtgtattaactaaattacgagatattagtttttgacactttttttctcgt

SEQ ID NO: 5 - DNA, 15111 bp, Human respiratory syncytial virus
  source 1..15111: mol_type = other DNA
acgggaaaaaatgcgtacaacaaacttgcataaaccaaaaaaatggggcaaataagaatttgataagtaccacttaaatttaactcccttggttagagatgggcagcaattcattgagtatgataaaagtcagattgcaaaatctattcgataatgacgaagtggcactattaaaaattacatgttataccgataaattgatacatctaactaatgcattagctaaagctgtaatacatacaattaaacttaatggaatagtgtttgtacatgtaattacatctagtgatatatgccctaataataatatcgtagtcaagtctaattttacaacaatgccagtgttacaaaatggcggatatatttgggaaatgatggaattgacacattgctcacaacctaatggtctattagacgataattgcgaaattaaatttagtaagaaattatccgatagtacaatgactaattatatgaatcaattatccgaattgttaggtttcgatcttaatccataaattataattaatatcaactagcaaatcaatgtcactaacaccattagttaatataaaacttaacagaagacaaaaatggggcaaataaatcaattcagccaacccaaccatggacacaacccacaatgataatacaccacaaagactgatgattaccgatatgagaccgttgtcacttgagacaattataactagcctaactagagatataataacacataaatttatatatctgattaatcacgaatgcatcgtgaggaaattggacgaaagacaggccacatttacattcttagtcaattacgaaatgaaactattgcataaggtaggctcaactaagtataagaaatataccgaatataacactaaatacggaacattcccaatgcctatattcataaatcacgacgggtttctcgaatgcataggcataaaacctacaaaacatacacccataatctataaatacgatcttaacccataaatttcaacacaatattcacacaatctaaaacaacaactctatgcataactatactccatagtccagatggagcctgaaaattatagtaatttaaaacttaaggagagatataagatagaagatggggcaaatacaaccatggctcttagcaaagtcaagttgaatgatacattgaataaagatcaattactatctagctcgaaatatactatccaacggtctacaggcgattcaatagatacacctaattacgatgtgcaaaaacatattaataaattgtgtggtatgttattgattaccgaagacgcaaatcataaatttacagggttaatcggtatgttatacgctatgtctagattaggtagggaagatacaattaaaatacttagagacgcaggatatcacgttaaagctaacggagtagacgtaactacacatagacaggatattaacggtaaggaaatgaaattcgaagtgttaacactcgctagcttaactaccgaaatacaaattaatatcgaaatcgaatcacgtaaatcttataagaaaatgcttaaagaaatgggcgaagtcgcacccgaatatagacacgatagtcccgattgtggtatgattatactatgtatagccgcattagtgataactaagttggccgcaggcgatagatccggattaaccgcagtgatacgtagagcgaataacgtacttaaaaacgaaatgaaacggtataagggtctattaccaaaagatatagcgaatagtttttacgaagtattcgaaaaacatccacattttatagacgtttttgtgcatttcggaatcgcacaatctagtactagaggaggatctagggttgagggtatattcgcaggattgtttatgaacgcatacggagcaggtcaagtcatgcttagatggggagtactcgcaaaatccgttaaaaatattatgttaggacacgctagcgtacaagccgaaatggaacaagtcgttgaggtatacgaatacgcacaaaaattaggtggagaagcaggattttatcatatactgaataatcctaaagctagtctattaagcttaacacaatttccacatttttctagcgtagtgttaggtaacgcagctggcctaggcataatgggcgaatataggggtacacctagaaatcaggatctatatgacgcagctaaagcatacgctgaacaattgaaagagaatggagtgataaattattccgtactcgatctaacagccgaagagttggaggcaattaaacatcaattgaatccgaaagataatgacgttgagttgtgagttaataaaaaatggggcaaataaatcatcatggaaaagtttgctcctgaattccatggagaagacgcaaataatagggcaacaaaattcttagagtcaatcaagggtaagtttacaagtccaaaagatccaaagaagaaagatagtataataagcgtaaactcaattgatatcgaggtgacaaaggaatcacctataacatctaatagtacaataataaatcccactaacgaaacagacgataccgcaggcaataaacctaattatcaacggaaacccttagtgtcattcaaagaagatccaacacctagtgataatccctttagtaaattgtataaggaaacaatcgaaacattcgataataacgaagaagaatcatcatactcatacgaagagataaacgatcagactaacgataatataaccgctagactagatagaatagacgaaaaactatctgaaatactaggtatgttacacacactagtagtcgcatctgccggacctacaagtgctagagatgggataagggatgcaatggtagggttaagggaagaaatgatagagaaaattagaaccgaagcattaatgactaacgatagactcgaagcaatggctagacttagaaacgaagaatccgaaaagatggcaaaagatacatctgacgaagtgtcacttaatcctactagcgaaaaattgaataatctattagagggaaacgatagtgataacgatctatcactcgaagatttctgattagttaccaatcttcacatcaacacacaataccaacagaagaccaacaaactaaccaacccaatcatccaaccaaacatccatccgccaatcagccaaacagccaacaaaacaaccagccaatccaaaactaaccacccggaaaaaatctataatatagttacaaaaaaaggaaagggtggggcaaatatggaaacatacgtgaacaagcttcacgaaggatcaacatatacagctgcagtccaatataacgtactcgaaaaagacgacgatcccgctagcctaacaatatgggtcccaatgtttcaatctagtatgcccgctgatctattaatcaaagaactagctaacgttaacatactagtcaaacaaattagtacacctaagggaccctcacttagagtgatgattaatagtagatccgcagtcctagcacaaatgcctagtaagtttacaatatgtgctaacgtaagcttagacgaacgatcaaaactagcatacgatgtgacaacaccatgcgaaatcaaagcatgttcattgacatgtcttaaatcaaagaatatgctaacaacagtcaaagatctaacaatgaaaacacttaatcccacacacgatataatcgcactatgcgaattcgaaaatatagtgactagtaagaaagtgataatccctacataccttagatcaatatccgttagaaataaggatctgaatacactcgaaaatataacaacaaccgaattcaaaaacgctataactaacgctaagataatcccttactccggactattgttagtgataaccgtaaccgataataagggagcattcaaatacataaaaccccaatcccaatttatagtcgatttaggcgcatacttagaaaaagaatcaatctattacgttacaactaattggaaacataccgctactagattcgcaatcaaacctatggaagattaacctttttcctctacatcagtgtgttaattcatacaaactttctacctacattcttcacttcaccatcacaatcacaaacactctgtggttcaaccaatcaaacaaaacttatctgaagtcccagatcatcccaagtcattgtttatcagatctagtactcaaataagttaataaaaaatatacacatggggcaaataatcattggaggaaatccaactaatcacaatatctgttaacatagacaagtccacacaccatacagaatcaaccaatggaaaatacatccataacaatagaattctctagcaaattttggccttactttacactaatacacatgataactacaatcatatccctattaatcataatctcaattatgatcgcaatccttaacaaactatgtgagtataacgtattccataacaaaacattcgaattgccaagagctcgagtgaatacctgataaagtagttaattaaaaatagtcataacaatgaactaggatatcaagactaacaataacattggggcaaatgcaaacatgtccaaaaacaaggaccaacgcaccgctaaaacactcgaaaggacatgggatacccttaatcacctattattcataagctcatgcttatataaattgaaccttaaatccgtcgcacagataaccctatcaatactcgcaatgataatctcaacaagcttaatcatagccgcaataatctttatcgctagcgctaaccataaggtaaccccaacaaccgcaattatacaggacgcaacatcccaaatcaaaaacacaaccccaacatacttaacccaaaacccacaactcggaatctcaccctctaacccatccgaaattacctcacagattacaacgatactcgcaagtacaacccccggagtcaaatcgacactccaatcgacaaccgtaaagactaagaatacaacaacaacccaaacccaacctagtaagcctacaactaagcaacgccaaaacaaacctccctctaaaccgaataacgattttcacttcgaagtgttcaatttcgtaccatgctcaatttgctctaataacccaacatgctgggccatatgcaaacgcatcccaaacaagaaacccggaaagaaaacaaccactaagccaacaaagaaaccaacccttaagacaaccaagaaagatccaaaaccccaaacaactaagtctaaagaggtcccaacaactaagccaaccgaagagccaacaatcaatacaactaagactaatataatcacaaccttactgacatctaacacaaccggaaatcccgaactgacatcccaaatggaaacctttcactcaacctctagcgaaggcaatccctcaccatcccaagtctcaaccactagcgaatacccatcccaacctagctcacctcccaatacccctagacagtagttacttaaaaacatattatcacaaaaggccttgaccaacttaaacagaatcaaaataaactctggggcaaataacaatggagttgctaatcctcaaagcaaatgcaatcacaacaatactaacagccgttacattttgtttcgctagcggacaaaacataaccgaagagttttatcaatctacatgttccgccgtaagtaaggggtatctatccgcacttagaaccggatggtatactagcgtaataacaatcgaactatctaatataaagaagaataagtgtaacggtacagacgctaaggttaaattgattaaacaggaactcgataagtataaaaacgccgtaaccgaattgcaattgttaatgcaatctacacaagctactaataatagggctagacgtgaattgcctagatttatgaattatacacttaataacgctaagaaaactaacgttacactatctaagaaacgaaaacgtagattcttagggtttttactcggagtcggttccgcaatcgctagcggagtcgccgtaagtaaagtgttacacctcgaaggcgaagtgaataagataaaatccgcactattatcaactaataaggcagtcgttagcctatctaacggagtcagcgtattgacatctaaagtgttagacttaaagaattatatagataagcaattgttaccaatcgttaataaacaatcatgttcaatatccaatatcgaaaccgtaatcgaatttcaacagaagaataatagattactcgaaattactagagaatttagcgtaaacgctggcgtaacaacacccgtaagtacatatatgttaactaattccgaactgttaagcttaattaacgatatgccaattactaacgatcagaagaaattgatgtctaataacgtacaaatcgttagacagcaatcatattcaattatgtcaattataaaagaagaggtactcgcatacgtagtgcaattacccctatatggcgtaatagatacaccatgttggaaattgcatacaagtccactatgtacaactaatacaaaagagggatctaatatatgcttaactagaaccgatagggggtggtattgcgataacgcaggtagcgtaagtttctttccacaagccgaaacatgtaaagtgcaatctaatagagtgttttgcgatacaatgaatagcttaacactacctagcgaagtcaatctatgtaacgtcgatatattcaatcctaaatatgattgcaaaattatgactagtaagactgacgtaagtagtagcgtaattactagtctcggtgcaatagtgtcatgttatggtaagactaagtgtaccgctagcaataagaatagggggataataaaaacatttagtaacggttgcgattacgttagtaataagggagtcgataccgtaagcgtaggtaatacactatattatgttaataaacaggaaggtaagtcattatacgttaaaggcgaacctataattaatttttacgatccattagtgtttccatccgacgaattcgacgctagtataagtcaggtaaacgaaaagattaaccaatcactcgcattcatacgaaaatccgacgaactgttacacaacgttaacgcaggtaagagtacaactaacataatgataacaacaattataatcgttataatcgttatactgttaagcttaatcgcagtcggattactgttatattgtaaagctagatcaacacccgtaacactatctaaagaccaattatccggtataaataatatcgcattctcaaactaaataaaaatagcacctaatcatgttcttacaatggtttactatctgctcatagacaacccatctgtcattggattttcttaaaatctgaacttcatcgaaactctcatctataaaccatctcacttacactatttaagtagattcctagtttatagttatataaaacacaattgcatgccagattaacttaccatctgtaaaaatgaaaactggggcaaatatgtcacgaaggaatccttgcaaatttgaaattcgaggtcattgcttaaatggtaagaggtgtcattttagtcataattattttgaatggccaccccatgcactgcttgtaagacaaaactttatgttaaacagaatacttaagtctatggataaaagtatagataccttatcagaaataagtggagctgcagagttggacagaacagaagagtatgctcttggtgtagttggagtgctagagagttatataggatcaataaacaatataactaaacaatcagcatgtgttgccatgagcaaactcctcactgaactcaatagtgatgatatcaaaaagctgagggacaatgaagagctaaattcacccaagataagagtgtacaatactgtcatatcatatattgaaagcaacaggaaaaacaataaacaaactatccatctgttaaaaagattgccagcagacgtattgaagaaaaccatcaaaaacacattggatatccataagagcataaccatcaacaacccaaaagaatcaactgttagtgatacaaatgaccatgccaaaaataatgatactacctgacaaatatccttgtagtataacttccatactaataacaagtagatgtagagttactatgtataatcaaaagaacacactatatttcaatcaaaacaacccaaataaccatatgtactcaccgaatcaaacattcaatgaaatccattggacctctcaagaattgattgacacaattcaaaattttctacaacatctaggtattattgaggatatatatacaatatatatattagtgtcataacactcaattctaacactcaccacatcgttacattattaattcaaacaattcaagttgtgggacaaaatggatcccattattaatggaaattctgctaacgtatacttaaccgatagttatttaaaaggcgtaatcagttttagcgaatgtaacgcattagggtcatatatctttaacggtccatatcttaaaaacgattatactaatctaatcagtagacagaatccgttaatcgaacatatgaatcttaagaaactgaatatcacacaatctttgatcagtaagtatcataaaggcgaaatcaaactcgaagaacctacatattttcaatcactattaatgacatataagtctatgacatctagcgaacagatcgctactactaatctgttgaagaaaattattagacgagctatagagatatctgacgttaaggtatacgctatactgaataaattggggttaaaagagaaagataagataaaatctaataacggtcaagacgaagataatagtgtaattactacaattattaaagacgatatactatccgcagtgaaggataatcaatcacatcttaaagccgataaaaatcatagtactaaacaaaaagatacaattaaaactacattgttaaagaaattgatgtgttctatgcaacatccacctagttggttaatacattggtttaacttatacactaagttgaacaatatacttacacaatatcgatcaaacgaagtgaaaaatcacggttttacattgatagataatcaaacattaagcggatttcaattcatacttaaccaatacggatgtatagtgtatcataaagaattgaaacgtataaccgttacaacatataatcaattcttaacatggaaagatataagtctatctagattgaacgtatgcttaattacatggatttcgaattgtcttaatacacttaataaatcattagggttaagatgcggatttaataacgttatacttacacaattgttcttatacggagattgtatacttaagttgttccataacgaagggttttatataataaaagaggttgagggatttataatgtcattgatactgaatattaccgaagaggatcaatttagaaaaagattctataatagtatgttaaacaatataactgacgcagctaataaagcgcagaagaatctgttatctagagtatgtcatacattgttagacaaaacagtgagcgataatattataaacggtagatggattatactgttatctaaattcttaaaattgattaagttggcaggtgacaataaccttaataacttaagcgaattgtatttcttattcagaatattcggacatcctatggttgacgaacgacaagctatggacgcagtgaagattaattgtaacgaaactaaattctatctattatctagtctatctatgcttagaggcgcattcatatatagaattataaaagggttcgttaataattataatagatggcctacacttagaaacgctatagtgttaccacttagatggttaacatattataaattgaatacatatcctagtttactcgaattaaccgaacgcgatctgatagtgttaagcggacttagattctatagagagtttagattgcctaagaaagtcgatctcgaaatgataattaacgataaggcaattagtccacctaaaaacttaatatggacaagcttccctagaaattatatgcctagtcatatacaaaattatatcgaacacgaaaaattgaaatttagcgaatccgataagtctagaagagtgttagagtattacttacgcgataataaatttaacgaatgcgatctatataattgcgtagtgaaccaatcatatcttaataatcctaatcacgtagtgagtcttacaggtaaggaaagagagttgagcgtaggtagaatgttcgctatgcaacccggtatgtttagacaagtgcaaatactcgcagaaaagatgatagccgaaaatatactgcaattctttcccgaatcattgactagatacggagatttagaattgcaaaagatactcgaattgaaagcaggtatatctaataagtctaatagatataacgataattataataattatatatctaagtgtagtattattaccgatctatctaaattcaatcaggcatttagatacgaaactagttgtatatgctcagacgtattagacgaattacacggagtgcaatctttgtttagttggttacatttaactatacctcacgttacaattatatgtacatatagacacgcaccaccatatataggcgatcatatagtcgatctgaataacgtagacgaacaatccggattgtatagatatcacatgggtggcatagagggatggtgtcaaaaattgtggactatagaggcaattagtctgttagatctaattagtcttaagggtaagttttcgattaccgcattgattaacggtgataatcaatcaattgatatatctaaaccgatacggttaatggagggacaaacacacgctcaagccgattacttactcgcacttaattcacttaaactgttatacaaagagtacgcaggtatagggcataaacttaagggtacagagacatatataagtagggatatgcaatttatgagtaagactatacaacataacggagtgtattatcccgctagtataaagaaagtgcttagagtcggaccttggattaatactatattagacgattttaaggttagtctcgaatcaatcggatcattgacacaagagttggagtatagaggcgaatctctattatgctcattgatttttagaaacgtatggttatacaatcagattgcattgcaattgaaaaatcacgcactatgtaataataagttgtacttagacatacttaaagtgttaaaacatcttaaaacattctttaatctcgataatatagataccgcattaacattgtatatgaatctacctatgttattcggagggggagatcctaatctattgtatagatcattctatagacgtacacctgatttcttaaccgaagctatagtgcatagcgtattcatactatcatattatactaatcacgatcttaaagataagttgcaggatctatctgacgatagattgaataaattcttaacatgtattataacattcgataaaaatcctaacgctgaattcgttacacttatgagagatccacaagcattaggttcagagagacaggctaaaattactagcgaaattaatagattagccgttaccgaagtgttaagtaccgcacctaataagatattctctaaatccgctcaacattatacaacaaccgaaatagatcttaacgatattatgcaaaatatcgaacctacatatcctcacggattacgcgtagtttacgaatcattaccattctataaagccgaaaagatcgttaacttaattagcggtacaaaatcaattactaatatactcgaaaagactagcgcaattgatttaaccgatatagatagagctaccgaaatgatgcgtaaaaatataacattactgatacgtatactaccattagattgtaatagggataaaagagagatactatctatggagaatctatcaattacagaattgtcaaaatacgttagggaacgatcatggtcactatctaatatcgtaggcgtaactagtcctagtattatgtatactatggatattaagtatacaactagtacaattagtagcggtataataatcgaaaaatataacgttaatagtctaacacgtggtgaaaggggacctacaaaaccttgggtcggatctagtacacaagagaagaaaactatgcccgtatataatagacaggtattgactaagaaacaacgagatcaaatagatctattagctaaactcgattgggtatacgctagtatagataataaagacgaatttatggaagagttgtcaatcggtacattagggttaacatacgaaaaagctaagaaattgttcccacaatatctatcagtgaattatctacatagattgacagtgagtagtagaccatgcgaatttcccgctagtatacccgcatatagaactactaattatcatttcgatactagtccaattaatagaatattaaccgaaaaatacggagacgaagatatagatatcgtattccaaaattgtattagtttcggattgagtcttatgtccgtagtcgaacaatttactaacgtatgtcctaataggattatactgatacctaaattgaacgaaatacatcttatgaaacctcctatttttacaggcgatgtcgatatacacaaattgaaacaggttatacaaaaacaacatatgttcttacccgataagatatcgttaacgcaatacgttgagttgttcttatcaaataaaacacttaaatcaggtagtcacgttaatagtaatctgatactcgcacataaaattagcgattactttcataatacatatatattgagtactaacttagccggacattggatactgattatacaattgatgaaagatagtaagggtatattcgaaaaagattggggtgagggatatataaccgatcatatgtttataaaccttaaggtcttctttaacgcatataaaacttatctattatgttttcataagggatacggtaaggctaaactcgaatgcgatatgaatacatccgatctattatgcgtactcgaattaattgatagtagctattggaaatctatgagtaaggtattcttagagcaaaaggtgatcaagtatatactatctcaagacgctagtttgcatagggttaagggatgtcatagttttaaattatggtttcttaaaagattgaacgtagccgaatttacagtatgtccttgggtcgttaacatagattatcatcctacacatatgaaagctatacttacatatatagatctagtgagaatgggattgattaacatagatagaatacatataaagaataaacataaatttaacgacgaattctatactagtaatctattctatataaattataatttttccgataatacacatctattaactaaacatatacgtatagctaatagcgaactcgaaaataattataataaattgtatcatcctacacccgaaacattagagaatatactcgctaatccgattaaatctaacgataagaaaacacttaacgattattgtataggtaaaaacgttgattcaattatgttaccattactatcaaataagaaattgattaaatctagcgctatgattagaactaattatagtaaacaggatctatataacttattccctatggtcgtaattgatagaattatagatcattccggtaataccgctaaatctaatcaattgtatacaactactagtcatcaaatatcattagtgcataatagtactagtctatattgtatgttaccatggcatcatattaatagattcaatttcgtttttagtagtacagggtgtaaaattagtatagagtatatacttaaagatcttaaaattaaagatcctaattgtattgcattcataggcgaaggcgcaggtaatctgttacttagaacagtagtcgaattgcatcccgatattagatatatatatagatcacttaaagattgtaacgatcatagtctaccaatcgaattccttagattgtataacggtcatataaacatagattacggcgaaaacttaacgatacccgctactgacgctactaataatatacattggtcatacttacatattaaattcgcagaacctataagtctattcgtatgcgacgcagaattatccgttacagtgaattggtctaaaattattatcgaatggtctaaacacgttagaaaatgcaaatattgttctagcgttaataagtgtatgttaatcgttaagtatcacgctcaagacgatatagattttaaattagataatataactatacttaaaacatacgtatgcttaggtagtaagcttaagggtagcgaagtatacttagtgttaacgataggtccagctaatatttttcccgtttttaacgtagtgcaaaacgctaaattgattctatctagaactaaaaattttataatgcctaagaaagctgataaagagtcaattgacgctaatataaaatcattgataccattcttatgttatcctataactaagaaagggattaataccgcactatctaaacttaaatccgtagtgagcggagatatactatcttatagtatagccggtagaaacgaagtttttagtaataaattgattaatcataaacatatgaatatacttaaatggtttaatcacgtacttaattttagatcaaccgaattgaattataatcatctatatatggtcgaatctacatatccatacttatccgaactgttaaactcattgactactaacgaattgaagaaattgattaaaattacaggtagtctgttatacaattttcataacgaataatgaataaagatcttataataaaaattcccatagctatacactaacactgtattcaattatagttattaaaaattaaaaatcatataattttttaaataacttttagtgaactaatcctaaagttatcattttaatcttggaggaataaatttaaaccctaatctaattggtttatatgtgtattaactaaattacgagatattagtttttgacactttttttctcgt

(19) United States
(12) Patent Application Publication
(10) Pub. No.: US 2024/0417699 A1
(43) Pub. Date: Dec. 19, 2024

(54) ATTENUATION OF HUMAN RESPIRATORY SYNCYTIAL VIRUS BY GENOME SCALE CODON-PAIR DEOPTIMIZATION

(71) Applicants: THE UNITED STATES OF AMERICA, AS REPRESENTED BY THE SECRETARY, DEPARTMENT OF HE, Bethesda, MD (US); The Research Foundation for the State University of New York, Albany, NY (US)

(72) Inventors: Peter L. COLLINS, Silver Spring, MD (US); Cyril LE NOUEN, Bethesda, MD (US); Linda G. BROCK, Bethesda, MD (US); Ursula J. BUCHHOLZ, Silver Spring, MD (US); Joshua Marc DINAPOLI, Lexington, MA (US); Steffen MUELLER, Kings Point, NY (US); Eckard WIMMER, E. Setauket, NY (US)

(21) Appl. No.: 18/756,709

(22) Filed: Jun. 27, 2024

Related U.S. Application Data
(63) Continuation of application No. 17/831,259, filed on Jun. 2, 2022, now Pat. No. 12,054,749.
Application No. 17/831,259 is a division of application No. 15/924,012, filed on Mar. 16, 2018, now Pat. No. 11,371,024.
Application No. 15/924,012 is a division of application No. 14/766,620, filed on Aug. 7, 2015, now Pat. No. 9,957,486.
(60) Provisional application No. 61/762,768, filed on Feb. 8, 2013.
(60) Provisional application No. 61/794,155, filed on Mar. 15, 2013.

(51) Int. Cl. C12N 7/00; A61K 39/00; A61K 39/155; C07K 14/005

(52) U.S. Cl. CPC C12N 7/00; A61K 39/155; C07K 14/005; A61K 2039/5254; A61K 2039/53; A61K 2039/55; C12N 2760/00021; C12N 2760/00034; C12N 2760/00062; C12N 2760/18522; C12N 2760/18534; C12N 2760/18561; C12N 2760/18562

(57) ABSTRACT

Described herein are RSV polynucleotide sequences that make use of multiple codons that are containing silent nucleotide substitutions engineered in multiple locations in the genome, wherein the substitutions introduce a numerous synonymous codons into the genome. Due to the large number of defects involved, the attenuated viruses disclosed herein provide a means of producing attenuated, live vaccines against RSV.

### CROSS-REFERENCE TO RELATED APPLICATIONS

This application is a Continuation Application of U.S. patent application Ser. No. 17/831,259, filed Jun. 2, 2022, which is a continuation U.S. patent application Ser. No. 15/924,012, filed Mar. 16, 2018, now U.S. Pat. No. 11,371,024, issued Jun. 28, 2022, which is a divisional application of U.S. patent application Ser. No. 14/766,620, filed Aug. 7, 2015, now U.S. Pat. No. 9,957,486, issued May 1, 2018, which is a national stage application under 35 U.S.C. 371 and claims the benefit of PCT Application No. PCT/US2014/015274 having an international filing date of Feb. 7, 2014, which designated the United States, which PCT Application claims priority to U.S. Provisional Application Ser. No. 61/794,155, filed Mar. 15, 2013 and U.S. Provisional Application Ser. No. 61/762,768, filed Feb. 8, 2013. All these applications are incorporated herein by reference in their entireties for all purposes.

### SEQUENCE LISTING

The instant application contains a Sequence Listing which has been submitted electronically in XML format and is hereby incorporated by reference in its entirety. Said XML copy, created on Jun. 26, 2024, is named Sequence_Listing_3000094-011005.xml and is 81,920 bytes in size.

### TECHNICAL FIELD

The subject matter disclosed herein relates to paramyxoviruses, in particular, respiratory syncytial virus and attenuated, mutant strains thereof.

### BACKGROUND

Human respiratory syncytial virus (RSV) infects nearly everyone worldwide early in life and is responsible for considerable mortality and morbidity. In the United States alone, RSV is responsible for 75,000-125,000 hospitalizations yearly, and worldwide conservative estimates conclude that RSV is responsible for 64 million pediatric infections and 160,000 pediatric deaths. Another unusual feature of RSV is that severe infection in infancy can be followed by years of airway dysfunction, including a predisposition to airway reactivity. RSV infection exacerbates asthma and may be involved in initiating asthma.

RSV is a member of the Paramyxoviridae family and, as such, is an enveloped virus that replicates in the cytoplasm and matures by budding through the host cell plasma membrane. The genome of RSV is a single, negative-sense strand of RNA of 15.2 kilobases that is transcribed by the viral polymerase into 10 mRNAs by a sequential stop-start mechanism that initiates at a single viral promoter at the 3′ end of the genome. Each mRNA encodes a single major protein, with the exception of the M2 mRNA, which has two overlapping open reading frames that encode two separate proteins. The 11 RSV proteins are: the RNA-binding nucleocapsid protein (N), the phosphoprotein (P), the large polymerase protein (L), the attachment glycoprotein (G), the fusion protein (F), the small hydrophobic (SH) surface glycoprotein, the internal matrix protein (M), the two nonstructural proteins NS1 and NS2, and the M2-1 and M2-2 proteins encoded by the M2 mRNA. The RSV gene order is: 3′-NS1-NS2-N-P-M-SH-G-F-M2-L. Each gene is flanked by short transcription signals called the gene-start (GS) signal, present on the upstream end of the gene and involved in initiating transcription of the respective gene, and the gene-end (GE) signal, present at the downstream end of the gene and involved in directing synthesis of a polyA tail followed by release of the mRNA.

Vaccines and new antiviral drugs are in pre-clinical and clinical development; however, no vaccines for RSV are commercially available yet. The goal of the present study was to design and generate new vaccine candidates for RSV by using the recently described synthetic attenuated virus engineering (SAVE) technique. (Coleman, et al., Science 320:1784-1787 (2008)). This technique is used to recode a genome in which the wild type (wt) amino acid sequence is unmodified, but synonymous codons are rearranged to create a suboptimal arrangement of pairs of codons that deviates from the natural frequency of occurrence of certain codon pairs. For pathogens, the attenuation resulting from this rearrangement of codons can be ‘titrated’ by adjusting the extent of codon-pair deoptimization (CPD). Recombinant pathogens that were attenuated by this approach encode proteins with wt aa sequences. Thus, these pathogens are likely to induce a cellular and humoral immunity against the same epitopes as the wt pathogen.

### SUMMARY

Described herein are RSV polynucleotide sequences that make use of multiple codons that are containing silent nucleotide substitutions engineered in multiple locations in the genome, wherein the substitutions introduce a numerous synonymous codon into the genome. This substitution of synonymous codons alters various parameters, including codon bias, codon pair bias, density of deoptimized codons and deoptimized codon pairs, RNA secondary structure, CpG dinucleotide content, C+G content, translation frameshift sites, translation pause sites, the presence or absence of tissue specific microRNA recognition sequences, or any combination thereof, in the genome. Because of the large number of defects involved, the attenuated virus of the invention provides a means of producing stably attenuated, live vaccines against RSV.

### BRIEF DESCRIPTION OF THE DRAWINGS

FIG. 1. Generation of synthetic codon-pair deoptimized rRSVs and characterization of their growth on Vero cells. (A) Four chimeric synthetic codon-pair deoptimized (CPD) rRSVs were generated based on the wt RSV backbone (Genbank Accession number M74568). Min A contained CPD ORFs of NS1, NS2, N, P, M and SH. Min B contained CPD ORFs of G and F. Min L contained a CPD ORF of polymerase protein L. Min FLC, all coding regions except M2, were codon pair deoptimized. CPD and wt coding sequences are represented by a black or grey shading boxes, respectively. (B) Multi-cycle growth kinetics of wt and codon-pair deoptimized rRSVs on Vero cells at 32 and 37° C. (C) Plaque size phenotype on Vero cells at 32° C. of rRSV and CPD rRSVs. (D) Specific infectivity of CPD rRSVs relative to rRSV was evaluated using strand-specific qRT-PCRs (Bessaud, M., et al., 2008. J Virol Methods 153:182-189). Viral RNAs derived from 4×10⁶ pfu of rRSV, Min A, Min L or Min FLC were extracted using a viral RNA extraction kit (Qiagen). Four microliters of the 60 μl RNA extraction were used in an RT reaction using superscript III reverse transcriptase and tagged primer containing an RSV-specific 3′-tail complementary to the RSV genomic RNA, and an unrelated 5′-tag sequence. Then, 10% of each cDNA reaction (corresponding to 2.7×10⁴ pfu) was used in the genome-specific qPCR described above. Results of the quantification of genomic RNA are expressed as fold difference compared to rRSV.

FIG. 2: Single cycle replication of CPD rRSVs compared to rRSV in Vero cells. Duplicate wells of confluent monolayer cultures of Vero cells in 6-well plates were mock-infected or infected at an MOI of 1 with rRSV, Min A, Min L and Min FLC and incubated at 32 or 37° C. Cultures were washed once after 2 h adsorption. Every four hours (from 4 to 24 h pi), viruses from one well were harvested and snap frozen for virus titration. Cells from a replica well were harvested for analysis of RNA and protein synthesis. Cell-associated RNA was used to specifically target and quantify (A) the virus antigenomic/mRNAs or (D) the RSV genomic RNA by a strand specific qPCR (Bessaud, M., et al., 2008. J Virol Methods 153:182-189). qPCR results were analyzed using the comparative threshold cycle (ΔCt) method, normalized to 18S rRNA and then for each virus expressed as fold increase over the 4 hour time point. (B, C) Ten micrograms of cell lysates were separated on NuPAGE 4-12% Bis-Tris SDS-PAGE gels and proteins were transferred to PVDF-F membranes. The membranes were blocked with Odyssey® blocking buffer and incubated with rabbit RSV-specific pAb and mouse α-tubulin used as a loading control. After washing, membranes were incubated with secondary antibodies goat anti-rabbit IgG IRDye 800 and goat anti-mouse IgG IRDye 680. Membrane strips were scanned on the Odyssey® Infrared Imaging System, background was corrected and fluorescence intensity of the N protein was evaluated using Odyssey® software, version 3.0 (Li-Cor). The 20 hour time point is shown in (D). (E) Virus titers from 4 to 24 hpi are expressed in log₁₀ pfu/ml. Virus aliquots were titrated in duplicate at the permissive temperature of 32° C. For each time point, the mean value of the duplicate is shown.

FIG. 3: Reduced replication of the CPD rRSVs in Balb/c mice. Six-week old Balb/c mice were inoculated intranasally in groups of ten with 4.5×10⁵ pfu of rRSV, Min A, Min L or Min FLC. At day 4 and 5, five mice per group were sacrificed. Nasal turbinates (NT) and lung tissue were harvested and homogenized separately. Virus titers were determined in duplicate by plaque assay on Vero cells incubated at 32° C. and expressed as log₁₀ pfu/gr. The median value is shown. Data sets were assessed for significance using non-parametric Kruskal-Wallis with Dunns post hoc test. Data were only considered significant at p<0.05 (*=p<0.05, **=p<0.01 and ***=p<0.001 compared to rRSV).

FIG. 4: Reduced replication of the CPD rRSVs in African Green Monkeys (AGM). AGM in groups of four were inoculated intranasally and intratracheally with 1×10⁶ pfu of Min A, Min L, Min FLC or rRSV per AGM. Nasopharyngeal (NP) swabs were collected every day from 0 to 12 days post inoculation and tracheal lavage (TL) samples were collected every other day from day 0 to 12. Virus titers in NP and TL were determined in duplicate on Vero cells incubated at 32° C. as described above. Sera were collected at day 0 and 28 and the 60% plaque reduction neutralizing-antibody titers (PRNT₆₀) were determined in a plaque reduction neutralization assay on Vero cells using GFP-expressing rRSV. Data sets were assessed for significance using non-parametric Kruskal-Wallis with Dunns post hoc test. Data were only considered significant at p<0.05 (*=p<0.05, **=p<0.01 compared to rRSV).

FIG. 5: Growth of Min L and Min FLC at increasing restrictive temperatures. Ten replicates cultures of Min L (A) and Min FLC (B) were grown serially at an increasing restrictive temperature starting at the shut off temperature (37 and 35° C. for Min L and Min FLC, respectively) with an input MOI of 0.1 pfu/cell until a maximum cytopathology was observed (between day 7 and 14). Two passages were performed for a given temperature before it was increased by 1° C. In parallel, as a control, duplicate samples of both viruses were grown on Vero cells in the same way but at 32° C. only (non-restrictive temperature). For each passage, 1 ml (out of 5 ml) of the supernatant was used to inoculate the next passage. For each passage, aliquots were frozen for titration and sequence analysis. Virus titer was determined in duplicate by plaque assay on Vero cells at the permissive temperature (32° C.). Passage number 0 represents the input virus. (C) The electropherograms show the nucleotide sequence of three mutations that were detected in N (aa 136), P (aa 114) and M2-1 (aa 88) of one replicate of Min L (gray filled triangle in (A)) which exhibited extensive syncytia at 39 and 40° C.

FIG. 6: Generation of synthetic codon-pair deoptimized rRSVs derived from Min L virus and characterization of their growth on Vero cells. (A) One chimeric synthetic codon-pair deoptimized (CPD) rRSV (Min L) was generated based on the wt RSV backbone (Genbank Accession number M74568). Min L contained a CPD ORF of polymerase protein L. CPD polymerase and wt coding sequences are represented by a black or grey shading boxes, respectively. Name of the genes as well as enzymatic restriction sites used for the generation of the chimeric synthetic cDNA are indicated. Mutations identified in N (aa 136), P (aa 114) and M2-1 (aa 88, cf FIG. 1) were introduced in all possible combinations. (B) Multi-cycle growth kinetics on Vero cells at 32 and 37° C. Confluent monolayer cultures of Vero cells in 6-well plates were infected in duplicate with rRSV, Min L or Min L containing the mutations of interest in all possible combinations at a multiplicity of infection (MOI) of 0.01 and incubated at 32 or 37° C. Viruses were harvested daily from day 1 to 14 by scraping infected cells into media followed by vortexing, clarification of the supernatant by centrifugation and freezing of virus aliquots. Each aliquot was titrated in duplicate at the permissive temperature of 32° C. For each time point, the mean value of the duplicate and standard deviation is shown. (C) Plaque size phenotype on Vero cells at 32° C. of rRSV and CPD rRSVs.

FIG. 7 shows an alignment of the genome sequences for wt RSV (SEQ ID NO: 1), RSV Min A (SEQ ID NO: 2), RSV Min B (SEQ ID NO: 3), RSV Min L (SEQ ID NO: 4), and RSV FLC (SEQ ID NO: 5).

### DETAILED DESCRIPTION OF ILLUSTRATIVE EMBODIMENTS

Provided herein are recombinant RSVs suitable for vaccine use in humans. Attenuated RSVs described herein are produced by introducing codon changes in the viral genome that are not optimally processed by the host cell. The majority of these mutations will not cause a change in the resulting amino acid of proteins encoded by the viral genome, thus allowing for the production of viruses that have the same antigenic features of wild-type viruses. It should be understood, however, that widespread noncoding changes to the codons of the viral genome may result in a selective pressure that gives rise to one or more amino acid mutations in the viruses described herein. Additionally, the described viruses may be combined with known attenuating mutations of RSV, of related viruses to yield an attenuated virus.

As used herein, the term “temperature sensitive” refers to the property of reduced replication compared to wild-type virus at temperatures at which the wild-type virus normally replicates. For example, wild-type RSV replicates efficiently within the range of 32° C. to 40° C., whereas a temperature-sensitive mutant would be restricted in replication at the higher temperatures within this range, but could be propagated efficiently at 32° C., which is called the “permissive temperature.” These viruses can be made using recombinant methods useful in identifying attenuated RSV strains. Once identified, the attenuating mutations can be introduced into biologically-derived strains, used to further attenuate or stabilize existing attenuated RSV strains, or attenuated RSV strains may be designed de novo.

The term “wild-type” as used herein refers to a viral phenotype that is consistent with efficient replication in a suitable permissive human host, and that may induce disease in a susceptible human host (for example, an RSV-naïve infant). The prototype A2 strain, represented by Genbank accession number M74568 but not strictly limited to that sequence, is considered to be an example of a wild-type strain. Derivative viruses that contain mutations that are presumed to not significantly reduce replication or disease in vivo also have the “wild-type” phenotype. In contrast, viral derivatives that exhibit reductions in replication of approximately 10-fold, 100-fold, or more in vivo may be considered to be “restricted”. Generally, restricted replication in vivo in a susceptible host is associated with reduced disease, or “attenuation.” Thus, infection of a susceptible host with an “attenuated” virus results in reduced disease in that host, as compared to a wild-type strain.

The term “parent” used in the context of a virus, protein, or polynucleotide denotes the virus, protein, or polynucleotide from which another virus is derived. The derived virus may be made by recombinant means, or by culturing the parent virus under conditions that give rise to a mutation, and thus a different virus. The term may refer to viral genomes and protein encoding sequences from which new sequences, which may be more or less attenuated, are derived. Parent viruses and sequences are usually wild type or naturally occurring prototypes or isolates of variants for which it is desired to obtain a more highly attenuated virus. However, parent viruses also include mutants specifically created or selected in the laboratory on the basis of real or perceived desirable properties. Accordingly, parent viruses that are candidates for attenuation include mutants of wild type or naturally occurring viruses that have deletions, insertions, amino acid substitutions and the like, and also include mutants which have codon substitutions.

The term “gene” or “gene sequence” refers to a polynucleotide sequence that encodes a protein and includes only the open reading frame portion of such a polynucleotide sequence.

### RSV Protein Sequences Encoded by Codon Deoptimized Polynucleotides

Described herein are RSV polynucleotide sequences that make use of multiple codons that are containing silent nucleotide substitutions engineered in multiple locations in the genome, wherein the substitutions introduce a numerous synonymous codon into the genome. This substitution of synonymous codons alters various parameters, including codon bias, codon pair bias, density of deoptimized codons and deoptimized codon pairs, RNA secondary structure, CpG dinucleotide content, C+G content, translation frameshift sites, translation pause sites, the presence or absence of tissue specific microRNA recognition sequences, or any combination thereof, in the genome. Because of the large number of defects involved, the attenuated virus of the invention provides a means of producing stably attenuated, live vaccines against RSV.

In one embodiment, an attenuated virus is provided which comprises a nucleic acid sequence encoding a viral protein or a portion thereof that is identical to the corresponding sequence of a parent virus, wherein the nucleotide sequence of the attenuated virus contains the codons of a parent sequence from which it is derived, and wherein the nucleotide sequence is less than 90% identical to the nucleotide sequence of the parent virus. In another embodiment, the nucleotide sequence is less than 80% identical to the sequence of the parent virus. In another embodiment, the nucleotide sequence is less than 70% identical to the sequence of the parent virus. The substituted nucleotide sequence which provides for attenuation is at least 100 nucleotides in length, or at least 250 nucleotides in length, or at least 500 nucleotides in length, or at least 1000 nucleotides in length. The codon pair bias of the attenuated sequence is less than the codon pair bias of the parent virus, and is reduced by at least about 0.05, or at least about 0.1, or at least about 0.2.

Described herein are codon pair deoptimized recombinant polynucleotides encoding a respiratory syncytial virus (RSV) amino acid sequence of a parent RSV, where the nucleotide sequence differs from the corresponding nucleotide sequence of the parent virus, resulting in a nucleotide identity of about 77% to about 93%. In some embodiments the amino acid sequence of the parent RSV may have an existing attenuating mutation, such that the resulting recombinant polynucleotide will encode the attenuating mutation to be encoded. The recombinant polynucleotides described herein, can encode any one of the RSV proteins NS1, NS2, N, P, M, SH, G, F, or L, or a combination of these proteins.

The recombinant polynucleotides described herein can encode the RSV NS1 protein. In one embodiment the nucleotide sequence encoding the RSV NS1 protein is from about 75% to about 95% identical to the corresponding nucleotide sequence of a parent virus. In one embodiment the nucleotide sequence encoding the RSV NS1 protein is from about 80% to about 90% identical to the corresponding nucleotide sequence of a parent virus. In one embodiment the nucleotide sequence encoding the RSV NS1 protein is about 87% identical to the corresponding nucleotide sequence of a parent virus. In one embodiment the nucleotide sequence encoding the RSV NS1 protein is from about 75% to about 95% identical to nucleotides 99 to 518 of SEQ ID NO: 5. In one embodiment the nucleotide sequence encoding the RSV NS1 protein is from about 80% to about 90% identical to nucleotides 99 to 518 of SEQ ID NO: 5. In one embodiment the nucleotide sequence encoding the RSV NS1 protein is about 87% identical to nucleotides 99 to 518 of SEQ ID NO: 5. In one embodiment the recombinant polynucleotide has the sequence of nucleotides 99 to 518 of SEQ ID NO: 5. In some embodiments the parent virus is an RSV subgroup A virus. In some embodiments the parent virus is an RSV subgroup B virus.

The recombinant polynucleotides described herein can encode the RSV NS2 protein. In one embodiment the nucleotide sequence encoding the RSV NS2 protein is from about 75% to about 95% identical to the corresponding nucleotide sequence of a parent virus. In one embodiment the nucleotide sequence encoding the RSV NS2 protein is from about 80% to about 90% identical to the corresponding nucleotide sequence of a parent virus. In one embodiment the nucleotide sequence encoding the RSV NS2 protein is about 88% identical to the corresponding nucleotide sequence of a parent virus. In one embodiment the nucleotide sequence encoding the RSV NS2 protein is from about 75% to about 95% identical to nucleotides 628 to 1002 of SEQ ID NO: 5. In one embodiment the nucleotide sequence encoding the RSV NS2 protein is from about 80% to about 90% identical to nucleotides 628 to 1002 of SEQ ID NO: 5. In one embodiment the nucleotide sequence encoding the RSV NS2 protein is about 88% identical to nucleotides 628 to 1002 of SEQ ID NO: 5. In one embodiment the recombinant polynucleotide has the sequence of nucleotides 628 to 1002 of SEQ ID NO: 5. In some embodiments the parent virus is an RSV subgroup A virus. In some embodiments the parent virus is an RSV subgroup B virus.

The recombinant polynucleotides described herein can encode the RSV N protein. In one embodiment the nucleotide sequence encoding the RSV N protein is from about 70% to about 90% identical to the corresponding nucleotide sequence of a parent virus. In one embodiment the nucleotide sequence encoding the RSV N protein is from about 75% to about 85% identical to the corresponding nucleotide sequence of a parent virus. In one embodiment the nucleotide sequence encoding the RSV N protein is about 80% identical to the corresponding nucleotide sequence of a parent virus. In one embodiment the nucleotide sequence encoding the RSV N protein is from about 70% to about 90% identical to nucleotides 1141 to 2316 of SEQ ID NO: 5. In one embodiment the nucleotide sequence encoding the RSV N protein is from about 75% to about 85% identical to nucleotides 1141 to 2316 of SEQ ID NO: 5. In one embodiment the nucleotide sequence encoding the RSV N protein is about 80% identical to nucleotides 1141 to 2316 of SEQ ID NO: 5. In one embodiment the recombinant polynucleotide has the sequence of nucleotides 1141 to 2316 of SEQ ID NO: 5. In some embodiments the parent virus is an RSV subgroup A virus. In some embodiments the parent virus is an RSV subgroup B virus.

The recombinant polynucleotides described herein can encode the RSVP protein. In one embodiment the nucleotide sequence encoding the RSV P protein is from about 75% to about 95% identical to the corresponding nucleotide sequence of a parent virus. In one embodiment the nucleotide sequence encoding the RSV P protein is from about 80% to about 90% identical to the corresponding nucleotide sequence of a parent virus. In one embodiment the nucleotide sequence encoding the RSV P protein is about 84% identical to the corresponding nucleotide sequence of a parent virus. In one embodiment the nucleotide sequence encoding the RSV P protein is from about 75% to about 95% identical to nucleotides 2347 to 3072 of SEQ ID NO: 5. In one embodiment the nucleotide sequence encoding the RSV P protein is from about 80% to about 90% identical to nucleotides 2347 to 3072 of SEQ ID NO: 5. In one embodiment the nucleotide sequence encoding the RSV P protein is about 84% identical to nucleotides 2347 to 3072 of SEQ ID NO: 5. In one embodiment the recombinant polynucleotide has the sequence of nucleotides 2347 to 3072 of SEQ ID NO: 5. In some embodiments the parent virus is an RSV subgroup A virus. In some embodiments the parent virus is an RSV subgroup B virus.

The recombinant polynucleotides described herein can encode the RSV M protein. In one embodiment the nucleotide sequence encoding the RSV M protein is from about 75% to about 95% identical to the corresponding nucleotide sequence of a parent virus. In one embodiment the nucleotide sequence encoding the RSV M protein is from about 80% to about 90% identical to the corresponding nucleotide sequence of a parent virus. In one embodiment the nucleotide sequence encoding the RSV M protein is about 83% identical to the corresponding nucleotide sequence of a parent virus. In one embodiment the nucleotide sequence encoding the RSV M protein is from about 75% to about 95% identical to nucleotides 3262 to 4032 of SEQ ID NO: 5. In one embodiment the nucleotide sequence encoding the RSV M protein is from about 80% to about 90% identical to nucleotides 3262 to 4032 of SEQ ID NO: 5. In one embodiment the nucleotide sequence encoding the RSV M protein is about 83% identical to nucleotides 3262 to 4032 of SEQ ID NO: 5. In one embodiment the recombinant polynucleotide has the sequence of nucleotides 3262 to 4032 of SEQ ID NO: 5. In some embodiments the parent virus is an RSV subgroup A virus. In some embodiments the parent virus is an RSV subgroup B virus.

The recombinant polynucleotides described herein can encode the RSV SH protein. In one embodiment the nucleotide sequence encoding the RSV SH protein is from about 85% to about 95% identical to the corresponding nucleotide sequence of a parent virus. In one embodiment the nucleotide sequence encoding the RSV SH protein is about 92% identical to the corresponding nucleotide sequence of a parent virus. In one embodiment the nucleotide sequence encoding the RSV SH protein is from about 85% to about 95% identical to nucleotides 4304 to 4498 of SEQ ID NO: 5. In one embodiment the nucleotide sequence encoding the RSV SH protein is about 92% identical to nucleotides 4304 to 4498 of SEQ ID NO: 5. In one embodiment the recombinant polynucleotide has the sequence of nucleotides 4304 to 4498 of SEQ ID NO: 5. In some embodiments the parent virus is an RSV subgroup A virus. In some embodiments the parent virus is an RSV subgroup B virus.

The recombinant polynucleotides described herein can encode the RSV G protein. In one embodiment the nucleotide sequence encoding the RSV G protein is from about 70% to about 90% identical to the corresponding nucleotide sequence of a parent virus. In one embodiment the nucleotide sequence encoding the RSV G protein is from about 75% to about 85% identical to the corresponding nucleotide sequence of a parent virus. In one embodiment the nucleotide sequence encoding the RSV G protein is about 78% identical to the corresponding nucleotide sequence of a parent virus. In one embodiment the nucleotide sequence encoding the RSV G protein is from about 70% to about 90% identical to nucleotides 4577 to 5473 of SEQ ID NO: 5. In one embodiment the nucleotide sequence encoding the RSV G protein is from about 75% to about 85% identical to nucleotides 4577 to 5473 of SEQ ID NO: 5. In one embodiment the nucleotide sequence encoding the RSV G protein is about 78% identical to nucleotides 4577 to 5473 of SEQ ID NO: 5. In one embodiment the recombinant polynucleotide has the sequence of nucleotides 4577 to 5473 of SEQ ID NO: 5. In some embodiments the parent virus is an RSV subgroup A virus. In some embodiments the parent virus is an RSV subgroup B virus.

The recombinant polynucleotides described herein can encode the RSV F protein. In one embodiment the nucleotide sequence encoding the RSV F protein is from about 70% to about 90% identical to the corresponding nucleotide sequence of a parent virus. In one embodiment the nucleotide sequence encoding the RSV F protein is from about 75% to about 85% identical to the corresponding nucleotide sequence of a parent virus. In one embodiment the nucleotide sequence encoding the RSV F protein is about 77% identical to the corresponding nucleotide sequence of a parent virus. In one embodiment the nucleotide sequence encoding the RSV F protein is from about 70% to about 90% identical to nucleotides 5550 to 7274 of SEQ ID NO: 5. In one embodiment the nucleotide sequence encoding the RSV F protein is from about 75% to about 85% identical to nucleotides 5550 to 7274 of SEQ ID NO: 5. In one embodiment the nucleotide sequence encoding the RSV F protein is about 77% identical to nucleotides 5550 to 7274 of SEQ ID NO: 5. In one embodiment the recombinant polynucleotide has the sequence of nucleotides 5550 to 7274 of SEQ ID NO: 5. In some embodiments the parent virus is an RSV subgroup A virus. In some embodiments the parent virus is an RSV subgroup B virus.

The recombinant polynucleotides described herein can encode the RSV L protein. In one embodiment the nucleotide sequence encoding the RSV L protein is from about 70% to about 90% identical to the corresponding nucleotide sequence of a parent virus. In one embodiment the nucleotide sequence encoding the RSV L protein is from about 75% to about 85% identical to the corresponding nucleotide sequence of a parent virus. In one embodiment the nucleotide sequence encoding the RSV L protein is about 79% identical to the corresponding nucleotide sequence of a parent virus. In one embodiment the nucleotide sequence encoding the RSV L protein is from about 70% to about 90% identical to nucleotides 8387 to 14884 of SEQ ID NO: 5. In one embodiment the nucleotide sequence encoding the RSV L protein is from about 75% to about 85% identical to nucleotides 8387 to 14884 of SEQ ID NO: 5. In one embodiment the nucleotide sequence encoding the RSV L protein is about 79% identical to nucleotides 8387 to 14884 of SEQ ID NO: 5. In one embodiment the recombinant polynucleotide has the sequence of nucleotides 8387 to 14884 of SEQ ID NO: 5. In some embodiments the parent virus is an RSV subgroup A virus. In some embodiments the parent virus is an RSV subgroup B virus.

The described deoptimized RSV polynucleotide sequences described herein can be used in combination with one another to form polynucleotides with two or more deoptimized gene sequences. In some embodiments the described polynucleotide may include any two codon deoptimized recombinant polynucleotides encoding RSV proteins selected from: NS1, NS2, N, P, M, SH, G, F, and L. In some embodiments the described polynucleotide may include any three codon deoptimized recombinant polynucleotides encoding RSV proteins selected from: NS1, NS2, N, P, M, SH, G, F, and L. In some embodiments the described polynucleotide may include any four codon deoptimized recombinant polynucleotides encoding RSV proteins selected from: NS1, NS2, N, P, M, SH, G, F, and L. In some embodiments the described polynucleotide may include any five codon deoptimized recombinant polynucleotides encoding RSV proteins selected from: NS1, NS2, N, P, M, SH, G, F, and L. In some embodiments the described polynucleotide may include any six codon deoptimized recombinant polynucleotides encoding RSV proteins selected from: NS1, NS2, N, P, M, SH, G, F, and L. In some embodiments the described polynucleotide may include any seven codon deoptimized recombinant polynucleotides encoding RSV proteins selected from: NS1, NS2, N, P, M, SH, G, F, and L. In some embodiments the described polynucleotide may include any eight codon deoptimized recombinant polynucleotides encoding RSV proteins selected from: NS1, NS2, N, P, M, SH, G, F, and L. In some embodiments the described polynucleotide may include nine codon deoptimized recombinant polynucleotides encoding RSV proteins: NS1, NS2, N, P, M, SH, G, F, and L. In some embodiments these described polynucleotides can be an RSV genome or antigenome having one or more codon deoptimized RSV gene sequences. The viruses RSV Min A, RSV Min B, RSV Min L, and RSV Min FLC have genome sequences representative of such virus constructs; however, other such viruses and viral genomes or antigenomes could be produced using the polynucleotide sequences provided herein.

The codon deoptimized sequences provided herein can also incorporate mutations to the amino acid sequence that are either derived from the parent gene sequence, are known to exist for the gene or protein encoded by the gene, or occur in the deoptimized gene de novo during the lifecycle of a virus having the deoptimized gene. In some embodiments the mutation can be a coding mutation, giving rise to a different amino acid residue in a given protein. In other embodiments, the mutation may occur in a gene having an unmodified, or parental sequence. For example, in one embodiment the mutation may occur in the codon encoding amino acid residue 136 of the N protein. In one embodiment the mutation in the codon encoding amino acid residue 136 of the N protein may cause an amino acid other than lysine to be encoded at position 136 of the N protein. In one embodiment the mutation in the codon encoding amino acid residue 136 of the N protein that causes an amino acid other than lysine to be encoded at position 136 of the N protein may occur in a virus having a codon deoptimized L protein, such as RSV Min L. In one embodiment the amino acid other than lysine encoded at position 136 of the N protein may be arginine. In one embodiment the mutation may occur in the codon encoding amino acid residue 114 of the P protein. In one embodiment the mutation in the codon encoding amino acid residue 114 of the P protein may cause an amino acid other than glutamic acid to be encoded at position 114 of the P protein. In one embodiment the mutation in the codon encoding amino acid residue 114 of the P protein that causes an amino acid other than glutamic acid to be encoded at position 114 of the P protein may occur in a virus having a codon deoptimized L protein, such as RSV Min L. In one embodiment the amino acid other than glutamic acid encoded at position 114 of the P protein may be valine. In one embodiment the mutation may occur in the codon encoding amino acid residue 88 of the M2-1 protein. In one embodiment the mutation in the codon encoding amino acid residue 88 of the M2-1 protein may cause an amino acid other than asparagine to be encoded at position 88 of the M2-1 protein. In one embodiment the mutation in the codon encoding amino acid residue 88 of the M2-1 protein that causes an amino acid other than asparagine to be encoded at position 88 of the M2-1 protein may occur in a virus having a codon deoptimized L protein, such as RSV Min L. In one embodiment the amino acid other than asparagine encoded at position 88 of the M2-1 protein may be lysine. In one embodiment the mutation may occur in the codon encoding amino acid residue 73 of the M2-1 protein. In one embodiment the mutation in the codon encoding amino acid residue 73 of the M2-1 protein may cause an amino acid other than alanine to be encoded at position 73 of the M2-1 protein. In one embodiment the mutation in the codon encoding amino acid residue 73 of the M2-1 protein that causes an amino acid other than alanine to be encoded at position 73 of the M2-1 protein may occur in a virus having a codon deoptimized L protein, such as RSV Min L. In one embodiment the amino acid other than alanine encoded at position 73 of the M2-1 protein may be serine.

### Methods of Producing Recombinant RSV

The ability to produce infectious RSV from cDNA permits the introduction of specific engineered changes, including site specific attenuating mutations, gene deletion, gene start sequence deletion or modification, and a broad spectrum of other recombinant changes, into the genome of a recombinant virus to produce an attenuated virus and, in some embodiments, effective RSV vaccine strains. Such engineered changes may, or may not, be based on biological mutations identified in other virus strains.

Described herein are infectious RSVs produced by recombinant methods, e.g., from cDNA. In one embodiment, infectious RSV is produced by the intracellular coexpression of a cDNA that encodes the RSV genomic RNA, together with those viral proteins necessary to generate a transcribing, replicating nucleocapsid, such as one or more sequences that encode major nucleocapsid (N) protein, nucleocapsid phosphoprotein (P), large (L) polymerase protein, and a transcriptional elongation factor M2-1 protein. Plasmids encoding other RSV, such as nonstructural protein 1 (NS1), nonstructural protein 2 (NS2), matrix protein (M), small hydrophobic protein (SH), glycoprotein (G), fusion protein (F), and protein M2-2, may also be included with these essential proteins. Accordingly, also described herein are isolated polynucleotides that encode the described mutated viruses, make up the described genomes or antigenomes, express the described genomes or antigenomes, or encode various proteins useful for making recombinant RSV in vitro. These polynucleotides can be included within or expressed by vectors in order to produce a recombinant RSV. Accordingly, cells transfected with the isolated polynucleotides or vectors are also within the scope of the invention and are exemplified herein. In addition, a number of methods relating to the described RSVs are also disclosed. For example, methods of producing the recombinant RSVs described herein are disclosed; as are methods producing an immune response to a viral protein in an animal, mammal or human.

The invention permits incorporation of biologically derived mutations, along with a broad range of other desired changes, into recombinant RSV vaccine strains. For example, the capability of producing virus from cDNA allows for incorporation of mutations occurring in attenuated RSV vaccine candidates to be introduced, individually or in various selected combinations, into a full-length cDNA clone, and the phenotypes of rescued recombinant viruses containing the introduced mutations to be readily determined. In exemplary embodiments, amino acid changes identified in attenuated, biologically-derived viruses, for example in a cold-passaged RSV (cpRSV), or in a further attenuated strain derived therefrom, such as a temperature-sensitive derivative of cpRSV (cptsRSV), are incorporated within recombinant RSV clones. These changes from a wild-type or biologically derived mutant RSV sequence specify desired characteristics in the resultant clones, e.g., an attenuated or further attenuated phenotype compared to a wild-type or incompletely attenuated parental RSV phenotype. In this regard, disclosed herein are novel RSV mutations that can be combined, either individually or in combination with one another, with preexisting attenuated RSV strains to produce viruses having desired characteristics, such as increased attenuation or enhanced genetic (and thereby phenotypic) stability in vitro and in vivo.

In addition to single and multiple point mutations and site-specific mutations, changes to recombinant RSV disclosed herein include deletions, insertions, substitutions or rearrangements of whole genes or gene segments. These mutations can affect small numbers of bases (e.g., from 15-30 bases, up to 35-50 bases or more), or large blocks of nucleotides (e.g., 50-100, 100-300, 300-500, 500-1,000 bases) depending upon the nature of the change (i.e., a small number of bases may be changed to insert or ablate an immunogenic epitope or change a small gene segment or delete one or more codons for purposes of attenuation, whereas large block(s) of bases are involved when genes or large gene segments are added, substituted, deleted or rearranged. These alterations will be understood by those of skill in the art based on prior work done with either RSV or related viruses. Viruses having block mutations of this sort can also be combined with the novel RSV mutations described herein, either individually or in combination with one another, to produce viruses having desired characteristics, such as increased attenuation or enhanced genetic (and thereby phenotypic) stability in vitro and in vivo.

In additional aspects, the invention provides for supplementation of mutations adopted from biologically derived RSV, e.g., cp and ts mutations, many of which occur in the L gene, with additional types of mutations involving the same or different genes or RNA signals in a recombinant RSV clone. RSV encodes ten mRNAs and eleven proteins. Three of these are transmembrane surface proteins, namely the attachment G protein, fusion F protein involved in penetration, and small hydrophobic SH protein. While specific functions may be assigned to single proteins, it is recognized that these assignments are provisional and descriptive. G and F are the major viral neutralization and protective antigens. Four additional proteins are associated with the viral nucleocapsid, namely the RNA binding protein N, the phosphoprotein P, the large polymerase protein L, and the transcription elongation factor M2-1. The matrix M protein is part of the inner virion and probably mediates association between the nucleocapsid and the envelope. Finally, there are two nonstructural proteins, NS1 and NS2, of unknown function. These proteins can be selectively altered in terms of its expression level, or can be added, deleted, substituted or rearranged, in whole or in part, alone or in combination with other desired modifications, in a recombinant RSV to obtain novel infectious RSV clones. In addition, the RNA genome contains cis-acting signals, including but not limited to the leader and trailer regions as well as the transcription gene-start (GS) and gene-end (GE) signals that border each gene. These signals help control encapsidation, transcription, and replication, and may have other roles as well. These signals can be selectively altered to obtain novel RSV clones.

The invention also provides methods for producing an infectious RSV from one or more isolated polynucleotides, e.g., one or more cDNAs. According to the present invention cDNA encoding a RSV genome or antigenome is constructed for intracellular or in vitro coexpression with the necessary viral proteins to form infectious RSV. By “RSV antigenome” is meant an isolated positive-sense polynucleotide molecule which serves as the template for the synthesis of progeny RSV genome. Preferably a cDNA is constructed which is a positive-sense version of the RSV genome, corresponding to the replicative intermediate RNA, or antigenome, so as to minimize the possibility of hybridizing with positive-sense transcripts of the complementing sequences that encode proteins necessary to generate a transcribing, replicating nucleocapsid, i.e., sequences that encode N, P, L and M2-1 protein.

A native RSV genome typically comprises a negative-sense polynucleotide molecule which, through complementary viral mRNAs, encodes eleven species of viral proteins, i.e., the nonstructural proteins NS1 and NS2, N, P, matrix (M), small hydrophobic (SH), glycoprotein (G), fusion (F), M2-1, M2-2, and L, substantially as described in Mink et al., Virology 185:615-624 (1991), Stec et al., Virology 183:273-287 (1991), and Connors et al., Virol. 208:478-484 (1995). For purposes of the present invention the genome or antigenome of the recombinant RSV of the invention need only contain those genes or portions thereof necessary to render the viral or subviral particles encoded thereby infectious. Further, the genes or portions thereof may be provided by more than one polynucleotide molecule, i.e., a gene may be provided by complementation or the like from a separate nucleotide molecule.

By recombinant RSV is meant a RSV or RSV-like viral or subviral particle derived directly or indirectly from a recombinant expression system or propagated from virus or subviral particles produced therefrom. The recombinant expression system will employ a recombinant expression vector which comprises an operably linked transcriptional unit comprising an assembly of at least a genetic element or elements having a regulatory role in RSV gene expression, for example, a promoter, a structural or coding sequence which is transcribed into RSV RNA, and appropriate transcription initiation and termination sequences.

To produce infectious RSV from cDNA-expressed genome or antigenome, the genome or antigenome is coexpressed with those RSV proteins necessary to (i) produce a nucleocapsid capable of RNA replication, and (ii) render progeny nucleocapsids competent for both RNA replication and transcription. Transcription by the genome nucleocapsid provides the other RSV proteins and initiates a productive infection. Additional RSV proteins needed for a productive infection can also be supplied by coexpression.

Alternative means to construct cDNA encoding the genome or antigenome include by reverse transcription-PCR using improved PCR conditions (e.g., as described in Cheng et al., Proc. Natl. Acad. Sci. USA 91:5695-5699 (1994); Samal et al., J. Virol 70:5075-5082 (1996)) to reduce the number of subunit cDNA components to as few as one or two pieces. In other embodiments, different promoters can be used (e.g., T3, SP6) or different ribozymes (e.g., that of hepatitis delta virus). Different DNA vectors (e.g., cosmids) can be used for propagation to better accommodate the large size genome or antigenome.

The N, P, L and M2-1 proteins are encoded by one or more expression vectors which can be the same or separate from that which encodes the genome or antigenome, and various combinations thereof. Additional proteins may be included as desired, encoded by its own vector or by a vector encoding a N, P, L, or M2-1 protein or the complete genome or antigenome. Expression of the genome or antigenome and proteins from transfected plasmids can be achieved, for example, by each cDNA being under the control of a promoter for T7 RNA polymerase, which in turn is supplied by infection, transfection or transduction with an expression system for the T7 RNA polymerase, e.g., a vaccinia virus MVA strain recombinant which expresses the T7 RNA polymerase (Wyatt et al., Virology, 210:202-205 (1995)). The viral proteins, and/or T7 RNA polymerase, can also be provided from transformed mammalian cells, or by transfection of preformed mRNA or protein.

To produce recombinant viruses having a codon deoptimized genome with the protein sequences described herein, one may use plasmids encoding an RSV genome having one or more gene sequences replaced with the corresponding codon deoptimized sequence provided herein. For example, the RSV genomes encoded by any one of SEQ ID NO: 2, SEQ ID NO: 3, SEQ ID NO: 4, and SEQ ID NO: 5 may be used in this manner. The constructs pRSV_Min A, pRSV_Min B, pRSV_Min L, pRSV_Min FLC, pRSV_Min L_N, pRSV_Min L_p, pRSV_Min L_M21, pRSV_Min L_NP, pRSV_Min L_NM21, pRSV_Min L_PM21, or pRSV_Min L_NPM21 provide examples in this regard.

Alternatively, synthesis of antigenome or genome can be done in vitro (cell-free) in a combined transcription-translation reaction, followed by transfection into cells. Or, antigenome or genome RNA can be synthesized in vitro and transfected into cells expressing RSV proteins.

### Uses of RSV Codon Deoptimized RSV Genes and Viruses

To select candidate vaccine viruses from the host of recombinant RSV strains capable of being produced using the codon deoptimized polynucleotide sequences provided herein, the criteria of viability, efficient replication in vitro, attenuation in vivo, immunogenicity, and phenotypic stability are determined according to well-known methods. Viruses which will be most desired in vaccines of the invention must maintain viability, must replicate sufficiently in vitro well under permissive conditions to make vaccine manufacture possible, must have a stable attenuation phenotype, must exhibit replication in an immunized host (albeit at lower levels), and must effectively elicit production of an immune response in a vaccine sufficient to confer protection against serious disease caused by subsequent infection from wild-type virus. Clearly, the heretofore known and reported RSV mutants do not meet all of these criteria. Indeed, contrary to expectations based on the results reported for known attenuated RSV, viruses of the invention are not only viable and more attenuated then previous mutants, but are more stable genetically in vivo than those previously studied mutants.

To propagate a RSV virus for vaccine use and other purposes, a number of cell lines which allow for RSV growth may be used. RSV grows in a variety of human and animal cells. Preferred cell lines for propagating attenuated RSV for vaccine use include DBS-FRhL-2, MRC-5, and Vero cells. Highest virus yields are usually achieved with epithelial cell lines such as Vero cells. Cells are typically inoculated with virus at a multiplicity of infection ranging from about 0.001 to 1.0, or more, and are cultivated under conditions permissive for replication of the virus, e.g., at about 30-37° C. and for about 3-10 days, or as long as necessary for virus to reach an adequate titer. Temperature-sensitive viruses often are grown using 32° C. as the “permissive temperature.” Virus is removed from cell culture and separated from cellular components, typically by well-known clarification procedures, e.g., centrifugation, and may be further purified as desired using procedures well known to those skilled in the art.

RSV which has been attenuated as described herein can be tested in various well known and generally accepted in vitro and in vivo models to confirm adequate attenuation, resistance to phenotypic reversion, and immunogenicity for vaccine use. In in vitro assays, the modified virus, which can be a multiply attenuated, biologically derived or recombinant RSV, is tested for temperature sensitivity of virus replication or “ts phenotype,” and for the small plaque phenotype. Modified viruses are further tested in animal models of RSV infection. A variety of animal models (e.g., murine, cotton rat, and primate) have been described and are known to those skilled in the art.

In accordance with the foregoing description and based on the Examples below, the invention also provides isolated, infectious RSV compositions for vaccine use. The attenuated virus which is a component of a vaccine is in an isolated and typically purified form. By isolated is meant to refer to RSV which is in other than a native environment of a wild-type virus, such as the nasopharynx of an infected individual. More generally, isolated is meant to include the attenuated virus as a component of a cell culture or other artificial medium. For example, attenuated RSV of the invention may be produced by an infected cell culture, separated from the cell culture and added to a stabilizer which contains other non-naturally occurring RSVs.

RSV vaccines of the invention contain as an active ingredient an immunogenically effective amount of RSV produced as described herein. Biologically derived or recombinant RSV can be used directly in vaccine formulations. The biologically derived or recombinantly modified virus may be introduced into a host with a physiologically acceptable carrier and/or adjuvant. Useful carriers are well known in the art, and include, e.g., water, buffered water, 0.4% saline, 0.3% glycine, hyaluronic acid and the like. The resulting aqueous solutions may be packaged for use as is, or lyophilized, the lyophilized preparation being combined with a sterile solution prior to administration, as mentioned above. The compositions may contain pharmaceutically acceptable auxiliary substances as required to approximate physiological conditions, which include, but are not limited to, pH adjusting and buffering agents, tonicity adjusting agents, wetting agents and the like, for example, sodium acetate, sodium lactate, sodium chloride, potassium chloride, calcium chloride, sucrose, magnesium sulfate, phosphate buffers, HEPES (4-(2-hydroxyethyl)-1-piperazineethanesulfonic acid) buffer, sorbitan monolaurate, and triethanolamine oleate. Acceptable adjuvants include incomplete Freund's adjuvant, aluminum phosphate, aluminum hydroxide, or alum, which are materials well known in the art. Preferred adjuvants also include Stimulon™ QS-21 (Aquila Biopharmaceuticals, Inc., Worchester, Mass.), MPL™ (3-0-deacylated monophosphoryl lipid A; RIBI ImmunoChem Research, Inc., Hamilton, Mont.), and interleukin-12 (Genetics Institute, Cambridge, Mass.).

Upon immunization with a RSV vaccine composition as described herein, via injection, aerosol, droplet, oral, topical or other route, the immune system of the host responds to the vaccine by producing antibodies specific for RSV virus proteins, e.g., F and G glycoproteins. As a result of the vaccination the host becomes at least partially or completely immune to RSV infection, or resistant to developing moderate or severe RSV disease, particularly of the lower respiratory tract.

The host to which the vaccine is administered can be any mammal susceptible to infection by RSV or a closely related virus and capable of generating a protective immune response to antigens of the vaccinizing strain. Thus, suitable hosts include humans, non-human primates, bovine, equine, swine, ovine, caprine, lagamorph, rodents, such as mice or cotton rats, etc. Accordingly, the invention provides methods for creating vaccines for a variety of human and veterinary uses.

The vaccine compositions containing the attenuated RSV of the invention are administered to a subject susceptible to or otherwise at risk of RSV infection in an “immunogenically effective dose” which is sufficient to induce or enhance the individual's immune response capabilities against RSV. An RSV vaccine composision may be administered to a subject via injection, aerosol delivery, nasal spray, nasal droplets, oral inoculation, or topical application. In the case of human subjects, the attenuated virus of the invention is administered according to well established human RSV vaccine protocols (Karron et al JID 191:1093-104, 2005). Briefly, adults or children are inoculated intranasally via droplet with an immunogenically effective dose of RSV vaccine, typically in a volume of 0.5 ml of a physiologically acceptable diluent or carrier. This has the advantage of simplicity and safety compared to parenteral immunization with a non-replicating vaccine. It also provides direct stimulation of local respiratory tract immunity, which plays a major role in resistance to RSV. Further, this mode of vaccination effectively bypasses the immunosuppressive effects of RSV-specific maternally-derived serum antibodies, which typically are found in the very young. Also, while the parenteral administration of RSV antigens can sometimes be associated with immunopathologic complications, this has never been observed with a live virus.

In all subjects, the precise amount of RSV vaccine administered and the timing and repetition of administration will be determined by various factors, including the patient's state of health and weight, the mode of administration, the nature of the formulation, etc. Dosages will generally range from about 10³ to about 10⁶ plaque forming units (“PFU”) or more of virus per patient, more commonly from about 10⁴ to 10⁵ PFU virus per patient. In one embodiment, about 10⁵ to 10⁶ PFU per patient could be administered during infancy, such as between 1 and 6 months of age, and one or more additional booster doses could be given 2-6 months or more later. In another embodiment, young infants could be given a dose of about 10⁵ to 10⁶ PFU per patient at approximately 2, 4, and 6 months of age, which is the recommended time of administration of a number of of other childhood vaccines. In yet another embodiment, an additional booster dose could be administered at approximately 10-15 months of age. In any event, the vaccine formulations should provide a quantity of attenuated RSV of the invention sufficient to effectively stimulate or induce an anti-RSV immune response (an “effective amount”). The resulting immune response can be characterized by a variety of methods. These include taking samples of nasal washes or sera for analysis of RSV-specific antibodies, which can be detected by tests including, but not limited to, complement fixation, plaque neutralization, enzyme-linked immunosorbent assay, luciferase-immunoprecipitation assay, and flow cytometry. In addition, immune responses can be detected by assay of cytokines in nasal washes or sera, ELISPOT of immune cells from either source, quantitative RT-PCR or microarray analysis of nasal wash or serum samples, and restimulation of immune cells from nasal washes or serum by re-exposure to viral antigen in vitro and analysis for the production or display of cytokines, surface markers, or other immune correlates measures by flow cytometry or for cytotoxic activity against indicator target cells displaying RSV antigens. In this regard, individuals are also monitored for signs and symptoms of upper respiratory illness. As with administration to chimpanzees, the attenuated virus of the vaccine grows in the nasopharynx of vaccinees at levels approximately 10-fold or more lower than wild-type virus, or approximately 10-fold or more lower when compared to levels of incompletely attenuated RSV.

In some embodiments, neonates and infants are given multiple doses of RSV vaccine to elicit sufficient levels of immunity. Administration may begin within the first month of life, and at intervals throughout childhood, such as at two months, four months, six months, one year and two years, as necessary to maintain sufficient levels of protection against natural RSV infection. In other embodiments, adults who are particularly susceptible to repeated or serious RSV infection, such as, for example, health care workers, day care workers, family members of young children, the elderly, individuals with compromised cardiopulmonary function, are given multiple doses of RSV vaccine to establish and/or maintain protective immune responses. Levels of induced immunity can be monitored by measuring amounts of neutralizing secretory and serum antibodies, and dosages adjusted or vaccinations repeated as necessary to maintain desired levels of protection. Further, different vaccine viruses may be indicated for administration to different recipient groups. For example, an engineered RSV strain expressing a cytokine or an additional protein rich in T cell epitopes may be particularly advantageous for adults rather than for infants. Vaccines produced in accordance with the present invention can be combined with viruses of the other subgroup or strains of RSV to achieve protection against multiple RSV subgroups or strains, or selected gene segments encoding, e.g., protective epitopes of these strains can be engineered into one RSV clone as described herein. In such embodiments, the different viruses can be in admixture and administered simultaneously or present in separate preparations and administered separately. For example, as the F glycoproteins of the two RSV subgroups differ by only about 11% in amino acid sequence, this similarity is the basis for a cross-protective immune response as observed in animals immunized with RSV or F antigen and challenged with a heterologous strain. Thus, immunization with one strain may protect against different strains of the same or different subgroup.

The vaccines of the invention elicit production of an immune response that may be protective against, or reduce the magnitude of serious lower respiratory tract disease, such as pneumonia and bronchiolitis when the individual is subsequently infected with wild-type RSV. While the naturally circulating virus is still capable of causing infection, particularly in the upper respiratory tract, there is a very greatly reduced possibility of rhinitis as a result of the vaccination and possible boosting of resistance by subsequent infection by wild-type virus. Following vaccination, there may be detectable levels of host engendered serum and, in some instances, secretory antibodies which are capable of neutralizing homologous (of the same subgroup) wild-type virus in vitro and in vivo. In many instances the host antibodies will also neutralize wild-type virus of a different, non-vaccine subgroup.

The level of attenuation of vaccine virus may be determined by, for example, quantifying the amount of virus present in the respiratory tract of an immunized host and comparing the amount to that produced by wild-type RSV or other attenuated RSVs which have been evaluated as candidate vaccine strains. For example, the attenuated virus of the invention will have a greater degree of restriction of replication in the upper respiratory tract of a highly susceptible host, such as a chimpanzee, compared to the levels of replication of wild-type virus, e.g., 10- to 1000-fold less. In order to further reduce the development of rhinorrhea, which is associated with the replication of virus in the upper respiratory tract, an ideal vaccine candidate virus should exhibit a restricted level of replication in both the upper and lower respiratory tract. However, the attenuated viruses of the invention must be sufficiently infectious and immunogenic in humans to confer protection in vaccinated individuals. Methods for determining levels of RSV in the nasopharynx of an infected host are well known in the literature. Specimens are obtained by aspiration or washing out of nasopharyngeal secretions and virus quantified in tissue culture or other by laboratory procedure. See, for example, Belshe et al., J. Med. Virology 1:157-162 (1977), Friedewald et al., J. Amer. Med. Assoc. 204:690-694 (1968); Gharpure et al., J. Virol. 3:414-421 (1969); and Wright et al., Arch. Ges. Virusforsch. 41:238-247 (1973). The virus can conveniently be measured in the nasopharynx of host animals, such as chimpanzees.

The following examples are provided by way of illustration, not limitation.

### Example 1: Design and Production of CPD RSVs

The sequences of all RSV open reading frames, except those encoding the M2-1 and M2-2 proteins, were codon pair deoptimized using previously described computational algorithms (Coleman et al., Science 320:1784-1787 (2008); Coleman et al., J Infect Dis 203:1264-1273 (2011)). Briefly, recombinant virus was recoded to use codon pairs that are underrepresented relative to the human genome. Constructs with underrepresented codon pairs were synthesized, sequenced and used to produce recombinant viruses. To preserve putative cis-acting signals and secondary structures present at the 5′ end of mRNAs, the original wt RSV nucleotide sequence was maintained for the first ten codons of each open reading frame. Runs of more than six identical nucleotides and RSV gene-end like or gene start like sequences were removed from the computer-generated CPD sequences by manual editing. The G/C content and the percentage of A, G, T, and C nucleotides, and of AT and GC dinucleotides, was similar between WT and CPD sequences. Percent nucleotide identity and number of nucleotide differences between WT and CPD RSV open reading frames are indicated in table 1. All nucleotide changes were silent on the amino acid level.

TABLE 1
Percent nucleotide identity and number
of mutations between WT and CPD
RSV open reading frames (ORF).

| | % | Number of |
|---|---|---|
| ORF | identity | mutations |
| NS1 | 87.8 | 65 |
| NS2 | 88.1 | 60 |
| N | 80.0 | 241 |
| P | 84.4 | 143 |
| M | 83.0 | 163 |
| SH | 92.3 | 23 |
| G | 78.7 | 197 |
| F | 77.8 | 422 |
| L | 79.1 | 1378 |

Recombinant (r)RSVs were constructed using a reverse genetic system based on strain A2 (Collins et al., Proc Natl Acad Sci U S A 92:11563-11567 (1995)). Recombinant viruses were constructed using the antigenome cDNA D46/6120, a derivative of the rA2 cDNA plasmid with a deletion of a 112-nt fragment of the downstream non-coding region of the SH gene. This cDNA exhibits improved stability during growth in E. coli. The changes in the SH noncoding region do not affect the efficiency of virus replication in vitro or in mice. Although the D46/6120 cDNA contains this deletion, for simplicity the numbering of sequence positions herein is based on the complete sequence of biologically derived strain A2 (Genbank accession number M74568) (SEQ ID NO: 1).

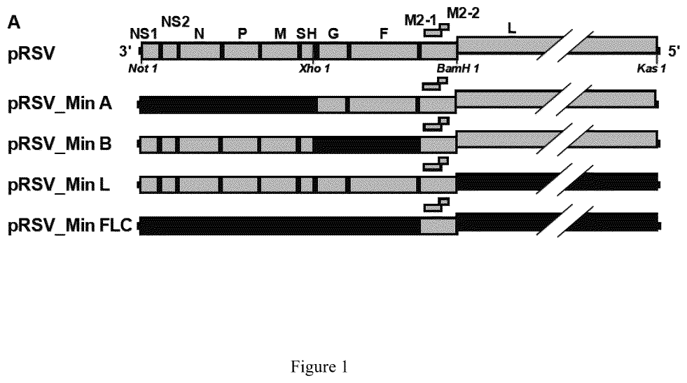

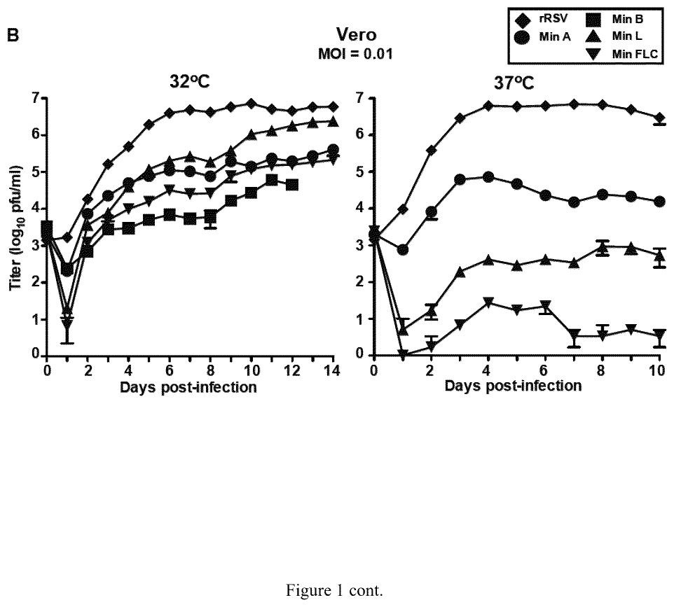

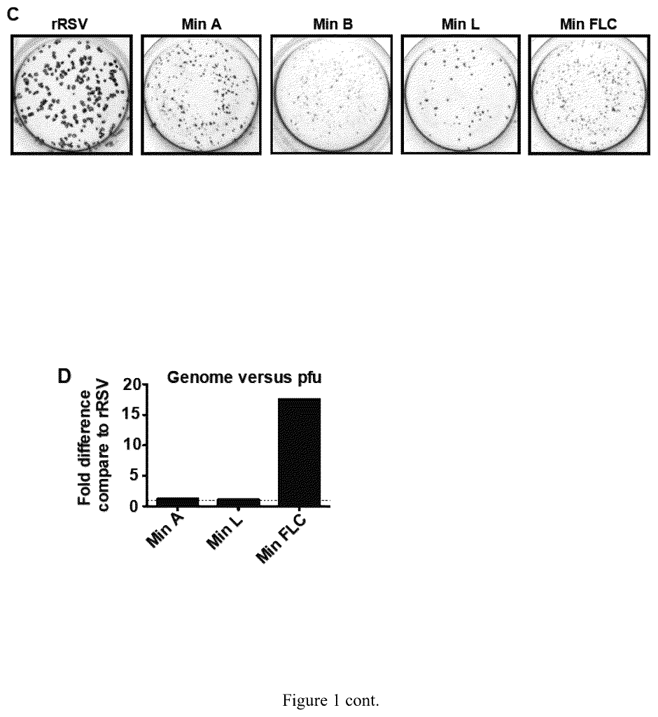

Four full-length cDNAs were generated, based on the D46/6120 backbone. These four cDNAs were named pRSV_Min A (SEQ ID NO: 2), pRSV_Min B (SEQ ID NO: 3), pRSV_Min L (SEQ ID NO: 4) and pRSV_Min FLC (SEQ ID NO: 5) (FIG. 1A). pRSV_Min A contains the CPD ORFs of NS1, NS2, N, P, M and SH. To construct pRSV_Min A, a 4508 base pair (bp) NotI-XhoI fragment of synthetic RSV cDNA with these six CPD ORFs was transferred into the similarly cleaved D46/6120 cDNA. pRSV_Min B contains the CPD G and F coding sequences that were transferred by cloning a 3907 bp XhoI-BamHI cDNA fragment with synthetic CPD ORFs into the similarly cleaved D46/6120 cDNA. pRSV_Min L contains a 6750 bp BamHI-KasI fragment with the CPD coding sequence of the L ORF that was transferred into the similarly cleaved D46/6120 cDNA. pRSV_Min FLC (FLC for full length clone) contained the entire RSV coding genes CPD. This full-length plasmid was generated by successively transferring all three synthetized CPD fragments (NotI-XhoI, XhoI-BamHI and BamHI-KasI) into the D46/6120 cDNA. After the generation of endo-free DNA preparations (Qiagen, Valencia, CA), the sequence of wt and of all four CPD plasmids were confirmed by sequence analysis of the RSV antigenome sequences contained in the cDNA plasmids.

To generate recombinant viruses from a wt RSV genome or the CPD RSV cDNA genomes (pRSV_Min A, pRSV_Min B, pRSV_Min L and pRSV_Min FLC) BSR T7/5 cells (a variant of baby hamster kidney 21 (BHK-21) cells constitutively expressing T7 RNA polymerase) were grown to 95% confluency in 6 well plates. Before transfection, cells were washed twice with GMEM containing 3% FBS, 1 mM 1-glutamine, and 2% MEM amino acids prior to the addition of 2 ml of media per well. Cells were transfected using Lipofectamine 2000 and a plasmid mixture containing 5 μg of full-length plasmid (FIG. 1A), 2 μg each of pTM1-N (encoding the wt RSV N protein) and pTM1-P (encoding the wt RSV N protein), and 1 μg each of pTM1-M2-1 (encoding the wt RSV N protein) and pTM1-L (encoding the wt RSV N protein). After overnight incubation at 37° C., transfected cells were harvested by scraping into media, added to subconfluent monolayers of Vero cells, and incubated at 32° C. The rescued viruses rRSV, rRSV_Min A (Min A), rRSV_Min B (Min B), rRSV_Min L (Min L) and rRSV_Min FLC (Min FLC) were harvested between 11 and 14 days post-transfection.

Following rescue, virus stocks were generated by scraping infected cells into media, followed by vortexing for 30 sec, clarification of the supernatant by centrifugation, and addition of 10× SPG (2.18 M sucrose, 0.038 M KH2PO4, 0.072 M K2HPO4, 0.06 M 1-glutamine at pH 7.1) to a final concentration of 1×. Virus aliquots were snap frozen and stored at −80° C. Virus titers were determined by plaque assay on Vero cells under 0.8% methylcellulose overlay. After a 10 to 12-day incubation at 32° C., plates were fixed with 80% cold methanol, and plaques were visualized by immunostaining with a cocktail of three RSV specific monoclonal antibodies. Titers were expressed as plaque forming unit (pfu) per ml. Viral RNA was isolated from all virus stocks, and sequence analysis of the viral genomes was performed using overlapping PCR fragments, confirming that the genomic sequences of the recombinant viruses were correct. The only sequences that were not directly confirmed for each genome were the positions of the outer-most primers, namely nt 1-29 and 1562-15089.

### Example 2: Kinetics of CPD rRSV Replication In Vitro

To assess the characteristics of the CPD RSVs multi-cycle and single cycle replication experiments were performed in Vero cells. In multi-cycle replication experiments, confluent monolayer cultures of Vero cells in 6-well plates were infected in triplicate at a multiplicity of infection (MOI) of 0.01 and incubated at 32 or 37° C. Viruses were harvested daily from day 1 to 14 (with the exception of Min B, the low stock titer of which only allowed to have time points from day 1 to 12 at 32° C.) by scraping infected cells into media followed by vortexing for 30 sec, clarification of the supernatant by centrifugation. Virus aliquots were snap frozen and stored at −80° C. until titration as described above. After all inoculations, the MOIs were confirmed by back-titration of the inoculum on Vero cells at 32° C.

At 32° C., the growth of all CPD viruses was delayed compared to rRSV; rRSV reached its maximum titer of 10⁷ pfu/ml on day 7, whereas Min A, Min L and Min FLC reached maximum titer on day 14. The maximum titer of Min L was only three-fold lower than that of rRSV. Min A and Min FLC were slightly more restricted in growth, as their maximum titers were around 15 and 30-fold lower than rRSV, respectively. Surprisingly, Min B was the most restricted for replication, as its maximum titer on day 12 was around 100-fold lower than that of rRSV. At 37° C., rRSV replicated faster than at 32° C., but it reached similar peak titers (about 10⁷ pfu/ml on day 4). At 37° C., the CPD viruses also reached their maximum titers earlier [at day 4 (Min A, Min FLC) or day 8 (Min L)]. Min A reached about the same maximum titers at 32° C. and at 37° C. (about 10⁵ pfu/ml). Surprisingly, despite the same amino acid sequence of all viruses, growth restriction at 37° C. was even greater than at 32° C. for Min L and Min FLC, which exhibited an about 100,000- and 1,000,000-fold restriction in growth compared to rRSV, respectively.

A plaque results from several cycles of virus replication, such that any differences in replication rates may be correlated to plaque size. Compared to rRSV, which induced large plaques on Vero cells at 32° C., Min A and Min L induced small plaques, and Min B and Min FLC induced micro-plaques (FIG. 1C).

### Example 3: Large-Extent Codon Pair Deoptimization Affects the Specific Infectivity of RSV

Experiments were conducted to assess the infectivity of the CPD viruses compared to rRSV using a strand-specific qRT-PCR designed to quantify viral genomic RNA (FIG. 1D). Viral RNA from virus stocks grown at 32° C. was purified using cell-free virus stocks with known titers. Genomic RNA corresponding to 2.7×10⁴ pfu of rRSV, Min A, Min L, and Min FLC virus stocks was quantified in Taqman® assays. In a typical experiment, Min A and Min L exhibited the same abundance of genomic RNA copies per 2.7×10⁴ plaque forming units as rRSV. However, the same amount of 2.7×10⁴ plaque forming units of the Min FLC virus stock contained 17.5-fold more genomic RNA than rRSV, Min A or Min L. This suggests that full length codon pair deoptimization of RSV greatly reduces its specific infectivity.

### Example 4: Temperature Sensitivity of CPD RSV

Studies were conducted to determine the shut off temperature (TSH) of the CPD viruses on Vero and Hep2 cells (Table 2). The T_{SH} is defined as the lowest restrictive temperature at which there is a reduction in virus titer compared to the permissive temperature 32° C. that is 100-fold or greater.

As expected, rRSV was not sensitive to temperature on both Vero and Hep2 cells. Confirming the results obtained from the multi-cycle replication experiment, replication of Min A was also not sensitive to temperature, as its T_{SH} was 40° C. on both Vero and Hep2 cells. Min B had a T_{SH} of 38° C. and 39° C. on Vero and Hep2 cells, respectively. Min L was more sensitive to temperature, with a T_{SH} of 37° C. and 38° C. on Vero and Hep2 cells, respectively. Finally, Min FLC was the most temperature sensitive virus, with a T_{SH} of 35° C. and 36° C. on Vero and Hep2 cells, respectively.

TABLE 2
Temperature sensitivity of the codon-pair
deoptimized RSVs on Hep2 and Vero cells.
Virus titer (in log₁₀ PFU per ml) at
indicated temperature (° C.) ^{a}

| | Cell | | | | | | | | |
|---|---|---|---|---|---|---|---|---|---|
| Virus | line | 32 | 35 | 36 | 37 | 38 | 39 | 40 | T_{SH} ^{b} |
| Min A | Vero | 7.2 | 6.6 | 6.2 | 6.1 | 5.7 | 5.4 | 4.4 | 40 |
| Min B | | 4.2 | 3.1 | 3.2 | 2.8 | 2.0 | 1.7 | <1 | 38 |
| Min L | | 7.5 | 7.1 | 6.5 | 5.2 | 4.2 | <1 | <1 | 37 |
| Min | | 6.7 | 4.5 | 3.4 | <1 | <1 | <1 | <1 | 35 |
| FLC | | | | | | | | | |
| WT | | 8.4 | 8.3 | 8.3 | 8.0 | 8.1 | 8.1 | 8.1 | >40 |
| Min A | HEp- | 7.1 | 6.7 | 6.6 | 6.4 | 6.3 | 5.6 | 4.8 | 40 |
| | 2 | | | | | | | | |
| Min B | | 4.0 | 4.0 | 4.0 | 4.0 | 3.7 | 2.0 | <1 | 39 |
| Min L | | 7.6 | 7.1 | 6.7 | 6.1 | 4.5 | 3.2 | 2.3 | 38 |
| Min | | 6.5 | 5.0 | 4.0 | 2.0 | <1 | <1 | < | 36 |
| FLC | | | | | | | | | |
| WT | | 8.3 | 8.1 | 8.3 | 8.3 | 8.1 | 8.3 | 8.1 | >40 |

^{a} The ts phenotype for each virus was evaluated by plaque assay on Vero and HEp-2 cells at the indicated temperatures. For viruses with a ts phenotype, the shut-off temperatures are marked (underlined). See footnote b for definition of shut-off temperature.
^{b} Shut off temperature (T_{SH}) is defined as the lowest restrictive temperature at which the reduction compared to 32° C. is 100-fold or greater than that observed for wt RSV at the two temperatures. The ts phenotype is defined as having a T_{SH} of 40° C. or less (bold).

### Example 5: Reduced Transcription of CPD rRSVs In Vitro

To investigate at which step(s) the growth restriction of the CPD viruses occurred, experiments were conducted to assess the viral mRNA/antigenome and genome synthesis, protein, and virus particle production in Vero cells in a single step replication experiment from 4 to 24 hours post-infection (hpi) at 32 and 37° C. Total RNA was used to quantify positive sense (antigenomic and mRNA) and negative sense (genomic) RSV RNA by strand specific Taqman® assays. Protein lysates were used to analyze viral protein synthesis by Western blot.

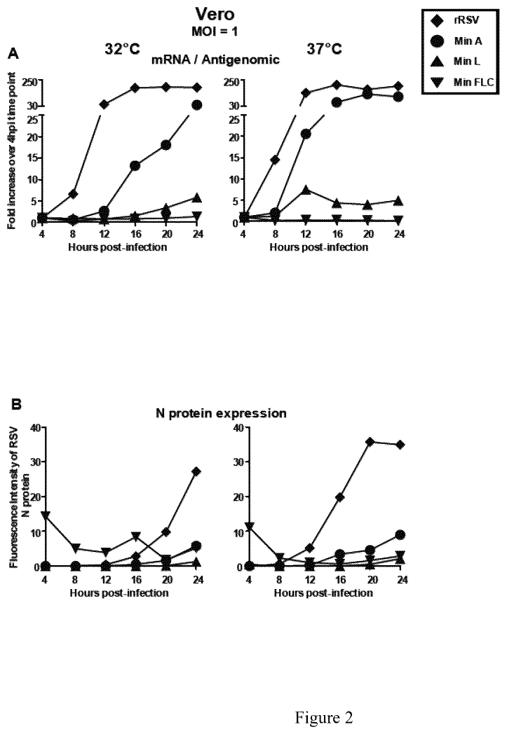

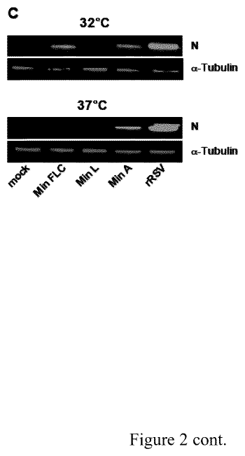

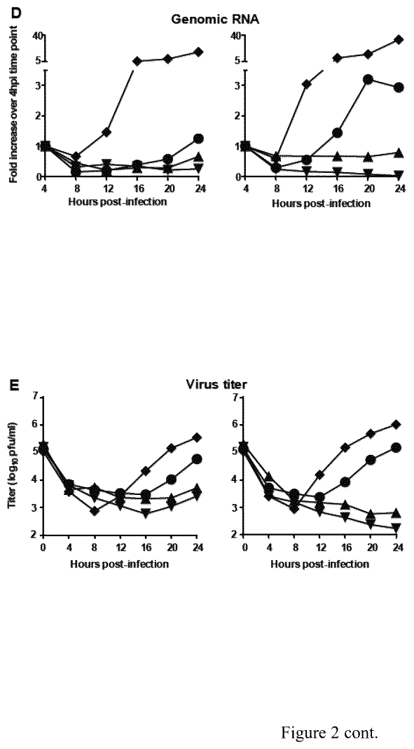

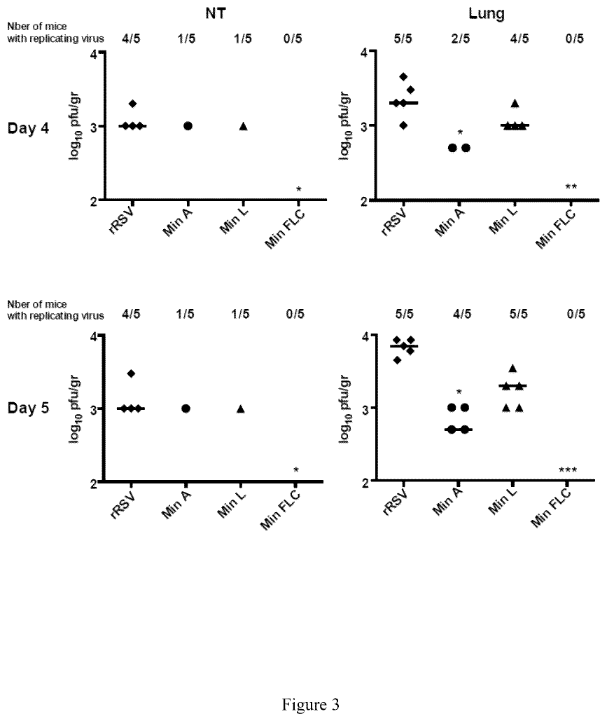

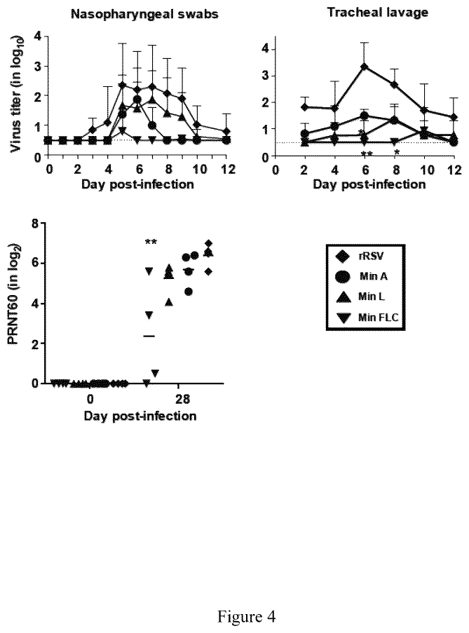

As shown in FIG. 2, rRSV exhibited strong transcription of viral genes as early as 8 hpi, and transcription was more efficient at 37° C. than at 32° C. (14- and 6-fold increase from 4 to 8 hpi at 37° C. and 32° C., respectively; FIG. 2A). Maximum gene transcription was reached at 16 hpi (188- and 196-fold increase of positive sense RNA at 32° C. or 37° C., respectively). Compared to rRSV, all CPD viruses exhibited reduced transcription. Min A gene transcription was delayed, and reduced compared to rRSV, at both 32° C. and 37° C. The first Min A transcripts were detected only at 12 hpi (3- and 21-fold increase of positive sense RNA from 4 to 12 hours at 32° C. and 37° C., respectively). Maximum Min A transcription was lower than that of rRSV at both 32° C. and 37° C. (a 41-fold increase of mRNA/antigenomic RNA from 4 to 24 hpi at 32° C., and a 129 fold increase at 20 hpi at 37° C.). Min L gene transcription was also reduced, as the first virus transcripts were detected only at 16 hpi at 32° C. at very modest levels (a 1.5-fold increase from 4 hpi to 16 hpi; maximum transcription at 24 hpi with only a 6 fold increase compared to 4 hpi). Transcription was not more efficient at 37° C., as an increase in positive-sense RNA was detectable starting at 16 hpi (a 4-fold increase compared to the 4 hour time point), with maximum transcription at 24 hpi (5 fold increase). Min FLC gene transcription was the least efficient, as a small increase of positive sense RNA was detected only 24 hpi at 32° C. (1.3-fold increase compared to 4 hpi), and was completely inefficient at 37° C., with no increase over time.

Reduced virus gene transcription in cells infected with the CDP viruses resulted in reduced protein synthesis. In rRSV infected cells, an increase in RSV N protein was detectable earlier (at 12 hpi and 16 hpi at 37° C. and 32° C., respectively) than in Min L and Min FLC infected cells; rRSV yielded higher levels of N protein expression than either of the three Min viruses at both 32 and 37° C. (FIG. 2B and C).

The production of genomic RNA paralleled the production of virus particles, and as expected was delayed compared to virus gene transcription. rRSV genomic RNA and virus particles were detected as early as 12 hpi and increased until 24 hours pi. All CPD viruses exhibited delayed and reduced synthesis of genomic RNA and virus particle production. Indeed, at 32° C., an increase of Min A genomic RNA and the first virus particles were detected at 20 hpi. At 37° C., Min A genomic RNA was detected at 12 hpi and the first virus particles were present at 16 hpi. Min L genomic RNA and particle release were also strongly delayed compared to rRSV. At 32 and 37° C., the first genomic RNAs as well as the first virus particles were detected at 24 hpi. Finally, at 32° C. Min FLC genomic RNA and virus particles were detected at 24 hpi, whereas no genomic RNA synthesis and virus particle release were detected at 37° C.

### Example 6: Replication of CPD rRSVs Are Reduced in Balb/c Mice

Replication of the CPD rRSVs compared to rRSV was evaluated in mice (FIG. 3). As expected, rRSV was detected in the nasal turbinates (NT) in 8 out of 10 mice on days 4 and 5 (median replication of 10³ pfu/g). Min A and Min L were only detected in the NT of a total of two out of 10 mice (one positive mouse on each day), whereas Min FLC was not detected in the NT of any mouse on any day (p<0.05 compared to rRSV). In the lungs, rRSV titer reached 10³ and 10⁴ pfu/g on days 4 and 5, respectively. Min A replication was significantly reduced compared to rRSV and was detected in only two mice on day 4. On day 5, 4 out of 5 mice exhibited virus replication. However, the titer was about 10-fold lower than that of rRSV (p<0.05). Min L replication was only slightly reduced as its titer was only two-fold lower than rRSV on day 4, and five-fold lower on day 5 (p>0.05). Finally, no Min FLC was detected in any mouse on day 4 or 5 (p<0.01 and p<0.001 at day 4 and 5, respectively), showing that this virus is strongly attenuated in mice. Taken together, these results show that Min A and Min FLC are attenuated in mice.

### Example 7: Replication of CPD rRSVs Are Reduced in African Green Monkeys

Studies were conducted to evaluate the replication of the CPD rRSVs in non-human primates (Table 3, 4 and FIG. 4). African green monkeys (AGM) in groups of four were inoculated intranasally and intratracheally with 1×10⁶ pfu of Min A, Min L, Min FLC or rRSV per AGM. Virus titers were evaluated from NP swabs and TL samples from 0 to 12 days.

While Min FLC did not replicate in the upper respiratory tract, virus shedding of Min A and Min L inoculated animals was slightly delayed and of shorter duration compared to rRSV inoculated animals. Peak titers of Min A and Min L were slightly lower than those of rRSV. In the lower respiratory tract, all CPD rRSV replicated poorly compared to rRSV (100-fold lower than rRSV; p<0.05). Despite strong restriction of replication of CPD rRSVs, Min A and Min L induced an efficient neutralizing antibody response (no significant difference between rRSV, Min A and Min L). However, as a consequence of inefficient replication, Min FLC was the only virus inducing a poor antibody response (p<0.05 compared to wt rRSV).

TABLE 3
Viral titers of nasal wash samples from nonhuman primates inoculated with Min A,
Min L, Min FLC or WT^{a}.

| | | | | | | | | | | | | | Duration | Peak | Sum of |
|---|---|---|---|---|---|---|---|---|---|---|---|---|---|---|---|
| | Animal | NW virus titer (log₁₀PFU/mL) on indicated days^{b} | | | | | | | | | | | of | virus | daily |
| Virus | ID | 1 | 2 | 3 | 4 | 5 | 6 | 7 | 8 | 9 | 10 | 12 | Shedding^{c} | titer | titers ^{d} |
| Min A | 7197 | — | — | — | — | — | 1.2 | 1.7 | — | — | — | — | 2 | 1.7 | 2.9 |
| | 7289 | — | — | — | — | 2.0 | 1.7 | — | — | — | — | — | 2 | 2.0 | 3.7 |
| | 7502 | — | — | — | — | — | 2.6 | 1.3 | — | — | — | — | 2 | 2.6 | 3.9 |
| | 7737 | — | — | — | 1.7 | 2.5 | 2.0 | — | — | — | — | — | 3 | 2.5 | 6.2 |
| | Mean: | | | | | | | | | | | | 2.3 | 2.2 | 4.2 |
| Min L | 7739 | — | — | — | — | 1.2 | — | 2.6 | 1.4 | 1.0 | — | 0.7 | 7 | 2.6 | 7.9 |
| | 7280 | — | — | — | — | 2.2 | 2.0 | 1.5 | 0.7 | — | — | — | 4 | 2.2 | 6.4 |
| | 7821 | — | — | — | — | — | — | 0.7 | — | 1.3 | 1.0 | — | 4 | 1.3 | 3.5 |
| | 7740 | — | — | — | — | 2.8 | 3.3 | 2.7 | 3.1 | 2.4 | — | — | 5 | 3.3 | 14.3 |
| | Mean: | | | | | | | | | | | | 5.0 | 2.4 | 8.0 |
| Min FLC | 7444 | — | — | — | — | — | — | — | — | — | — | — | 0 | <0.5 | — |
| | 7431 | — | — | — | — | 1.7 | — | — | — | 0.7 | — | — | 5 | 1.7 | 3.9 |
| | 7798 | — | — | — | — | — | — | — | — | — | — | — | 0 | <0.5 | — |
| | 7632 | — | — | — | — | — | — | — | — | — | — | — | 0 | <0.5 | — |
| | Mean: | | | | | | | | | | | | 1.3 | 0.4 | 1.0 |
| WT | 7489 | — | — | 1.2 | 2.9 | 3.3 | 3.5 | 2.0 | 3.3 | 3.0 | 1.8 | — | 8 | 3.5 | 21.0 |
| | 7538 | — | — | 1.2 | — | 2.0 | 2.8 | 3.0 | 2.5 | 2.2 | — | — | 7 | 3.0 | 14.2 |
| | 7370 | — | — | — | — | — | 2.0 | — | — | 1.9 | 1.3 | 1.7 | 6 | 2.0 | 7.9 |
| | 7667 | — | — | — | — | 3.6 | — | 3.7 | 2.0 | — | — | — | 4 | 3.7 | 9.8 |
| | Mean: | | | | | | | | | | | | 6.3 | 3.1 | 13.2 |

^{a}Nonhuman primates were inoculated by the combined intranasal and intratracheal routes with 10⁶ PFU of the indicated virus in a 1 ml inoculum per site (total dose = 2 × 10⁶ PFU per animal).
^{b}Nasal wash was performed with 2 ml of Leibovitz L-15 medium with 1× SP used as stabilizator. Virus titrations were performed on Vero cells at 32° C. The lower limit of detection was 0.7 log₁₀ PFU/ml of nasal wash solution. Samples with no detectable virus are represented as “—“. Peak titers for each animal are underlined.
^{c}The period of days from the first to the last day on which virus was detected, including negative days (if any) in between.
^{d} The sum of daily titers is used as an estimate for the magnitude of shedding (area under the curve). A value of 0.5 was used for samples with no detectable virus.

TABLE 4
Viral titers of bronchoalveolar and tracheal lavage samples from nonhuman
primates inoculated with Min A, Min L, Min FLC or WT ^{a}.

| | | Tracheal Lavage virus titer | | | | | | | | |
|---|---|---|---|---|---|---|---|---|---|---|
| | | (log₁₀ PUF/ml) on indicated days^{b} | | | | | | Duration of | Peak | Sum of daily |
| Virus | Animal ID | 2 | 4 | 6 | 8 | 10 | 12 | Shedding^{c} | virus titer | titers ^{d} |
| Min A | 7197 | 1.3 | — | 1.3 | 1.3 | — | — | 7 | 1.3 | 4.9 |
| | 7289 | 1.0 | 1.3 | 1.6 | — | 1.6 | — | 9 | 1.6 | 6.5 |
| | 7502 | — | 2.1 | 1.8 | 1.5 | — | — | 6 | 2.1 | 5.4 |
| | 7737 | — | — | 1.3 | 2.0 | — | — | 3 | 2.0 | 3.3 |
| | Mean: | | | | | | | 6.3 | 1.8 | 5.0 |
| Min L | 7739 | — | 1.5 | — | 2.0 | — | 1.6 | 9 | 2.0 | 7.1 |
| | 7280 | — | — | — | 1.0 | — | — | 1 | 1.0 | 1.0 |
| | 7821 | — | — | — | 1.0 | — | — | 1 | 1.0 | 1.0 |
| | 7740 | — | — | 1.6 | 1.5 | 1.6 | — | 5 | 1.6 | 4.7 |
| | Mean: | | | | | | | 4.0 | 1.4 | 3.5 |
| Min FLC | 7444 | — | — | — | — | — | — | 0 | <1.0 | — |
| | 7431 | — | — | — | — | — | — | 0 | <1.0 | — |
| | 7798 | — | — | — | — | 2.3 | — | 1 | 2.3 | 2.3 |
| | 7632 | — | — | — | — | — | — | 0 | <1.0 | — |
| | Mean: | | | | | | | 0.3 | 1.3 | 0.6 |
| WT | 7489 | 2.3 | — | 2.6 | 3.3 | 2.6 | 1.5 | 11 | 3.3 | 13.3 |
| | 7538 | 1.5 | 1.5 | 2.5 | 2.7 | 2.4 | 2.3 | 11 | 2.7 | 12.9 |
| | 7370 | 2.0 | 2.2 | 4.2 | 1.9 | 1.3 | 1.5 | 11 | 4.2 | 13.1 |
| | 7667 | 1.5 | 2.9 | 4.1 | 2.9 | — | — | 7 | 4.1 | 11.4 |
| | Mean: | | | | | | | 10.0 | 3.6 | 12.7 |

^{a} Nonhuman primates were inoculated by the combined intranasal and intratracheal routes with 10⁶ PFU of the indicated virus in a 1 ml inoculum per site (total dose = 2 × 10⁶ PFU per animal).
^{b}On days 2, 4, 6, and 8, bronchoalveolar lavage was performed with 3 ml of PBS and mixed 1:1 with L15 mediu with 2× SP. Virus titrations were performed on Vero cells at 32° C. The lower limit of detection was 1.0 log₁₀ PFU/mL of lavage solution. Samples with no detectable virus are represented as “—“. Peak titers for each animal are underlined.
cThe period of days from the first to the last day on which virus was detected, including negative days (if any) in between.
^{d} The sum of daily titers is used as an estimate for the magnitude of shedding (area under the curve). A value of 1.0 was used for samples with no detectable virus.

### Example 8: Growth of Min L and Min FLC at Increasing Restrictive Temperatures

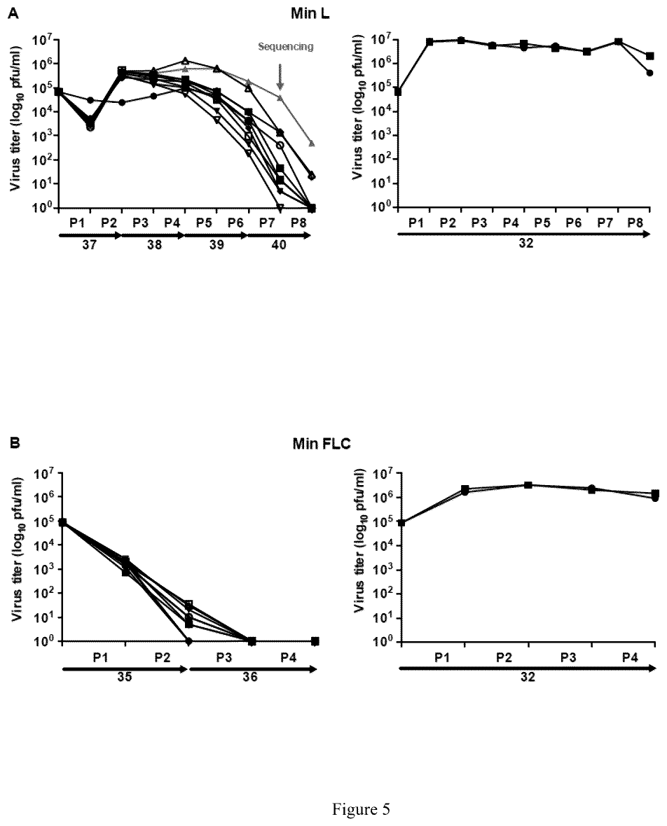

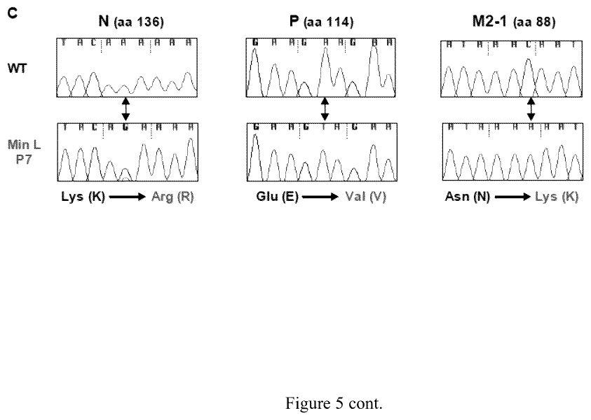

Ten replicates cultures of Min L (FIG. 5A) and Min FLC (FIG. 5B) were grown serially at an increasing restrictive temperature starting at the shut off temperature (37 and 35° C. for Min L and Min FLC, respectively) with an input MOI of 0.1 pfu/cell until a maximum cytopathology was observed (between day 7 and 14). Two passages were performed for a given temperature before it was increased by 1° C. In parallel, as a control, duplicate samples of both viruses were grown on Vero cells in the same way but at 32° C. only (non-restrictive temperature). For each passage, 1 ml (out of 5 ml) of the supernatant was used to inoculate the next passage. For each passage, aliquots were frozen for titration and sequence analysis. Virus titer was determined in duplicate by plaque assay on Vero cells at the permissive temperature (32° C.). Sequence analysis revealed three mutations in a single Min L replicate (FIG. 5C). The N protein had a mutation in the codon encoding amino acid 136, resulting in a change in the encoded amino acid from lysine to arginine. The P protein had a mutation in the codon encoding amino acid 114, resulting in a change in the encoded amino acid from glutamic acid to valine. Also the M2-1 protein had a mutation in the codon encoding amino acid 88, resulting in a change in the encoded amino acid from asparagine to lysine. The Min L replicate with these mutations exhibited extensive syncytia at 39 and 40° C. (red filled triangle in (FIG. 5A)).

### Example 9: Generation of Synthetic Codon-pair Deoptimized rRSVs Derived From Min L Virus and Characterization of Their Growth on Vero Cells

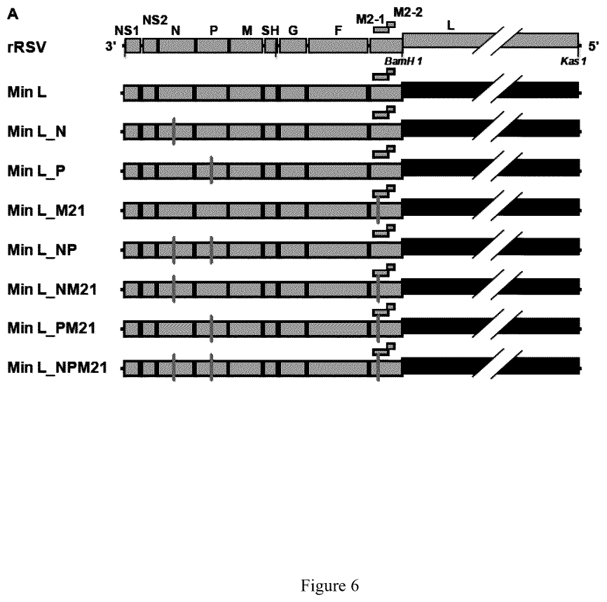

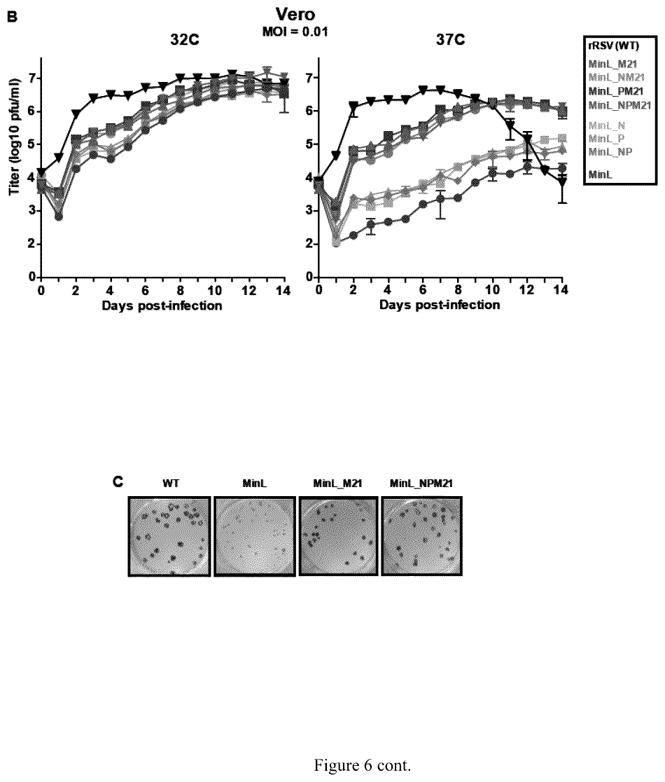

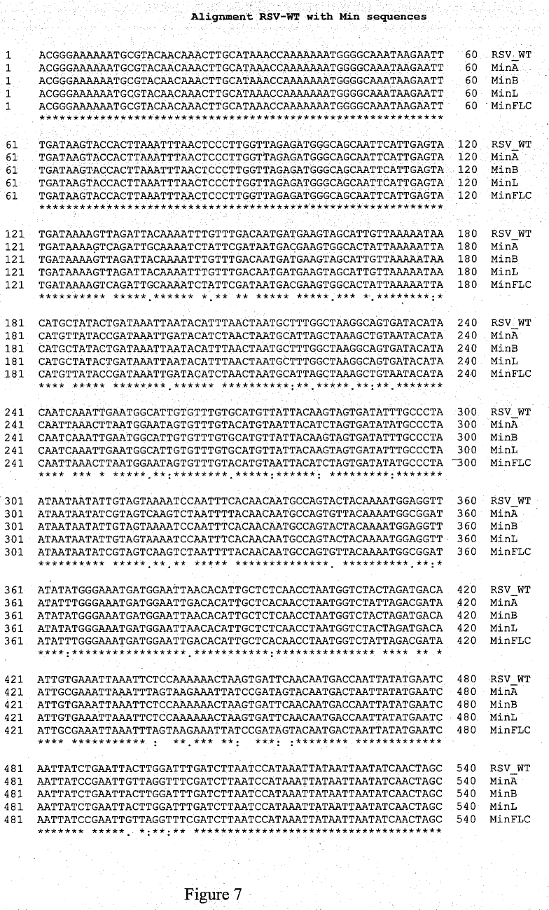

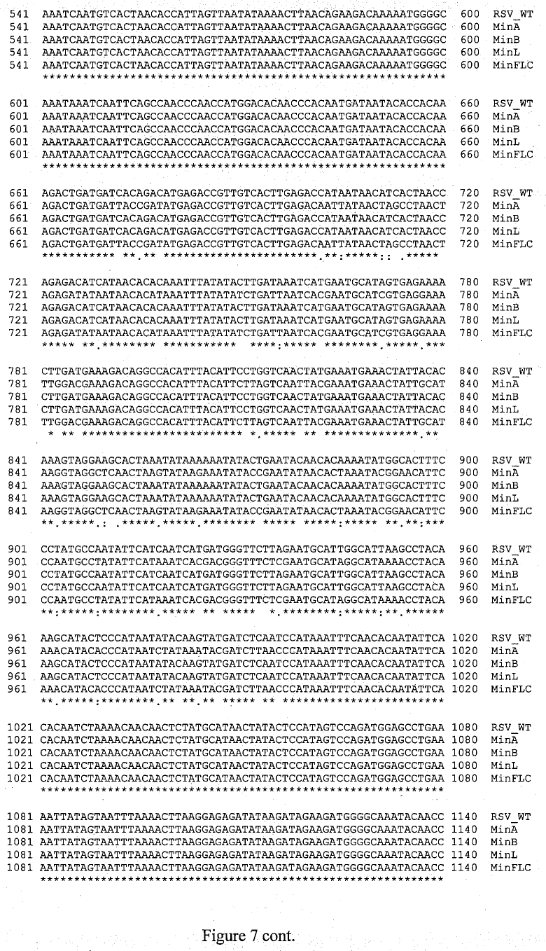

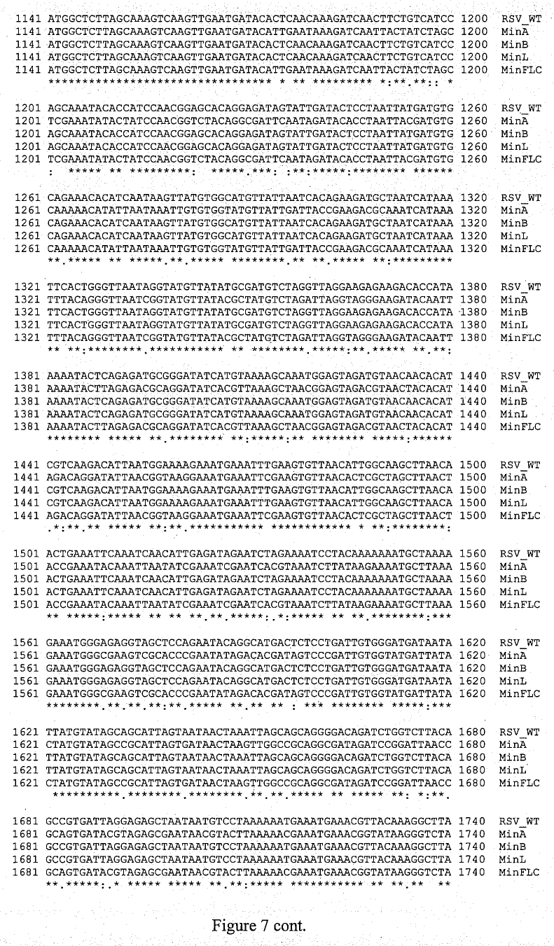

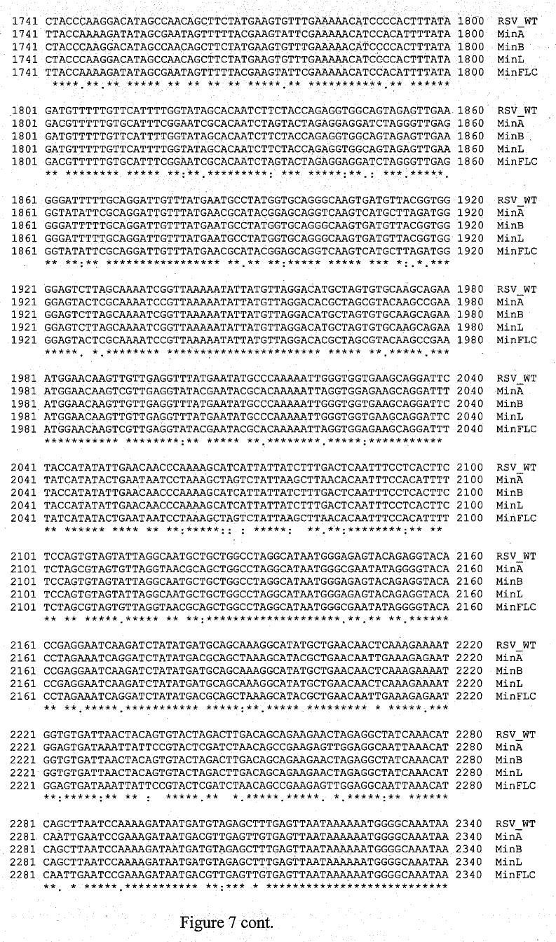

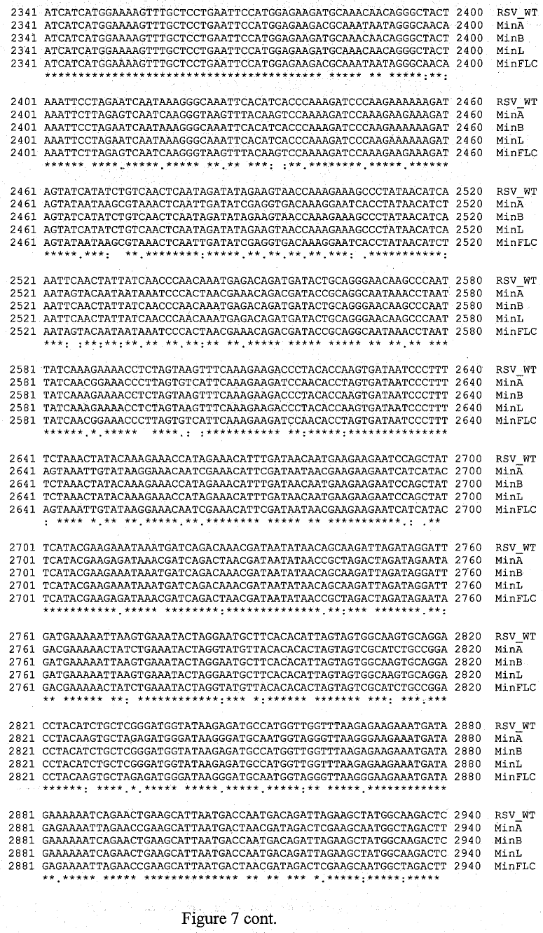

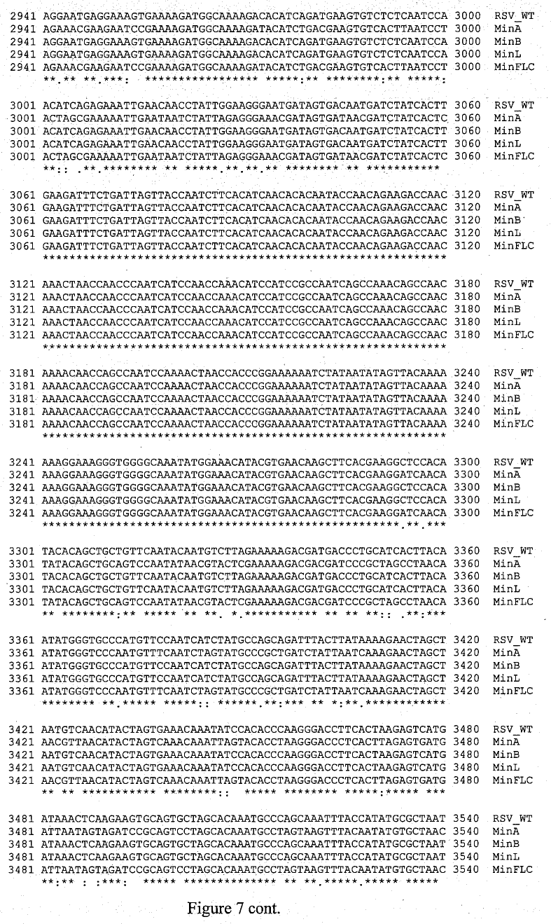

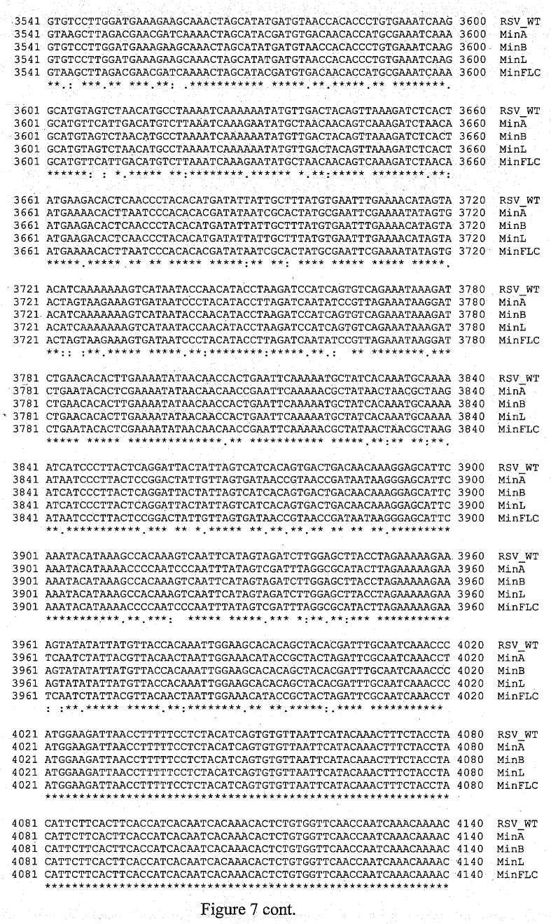

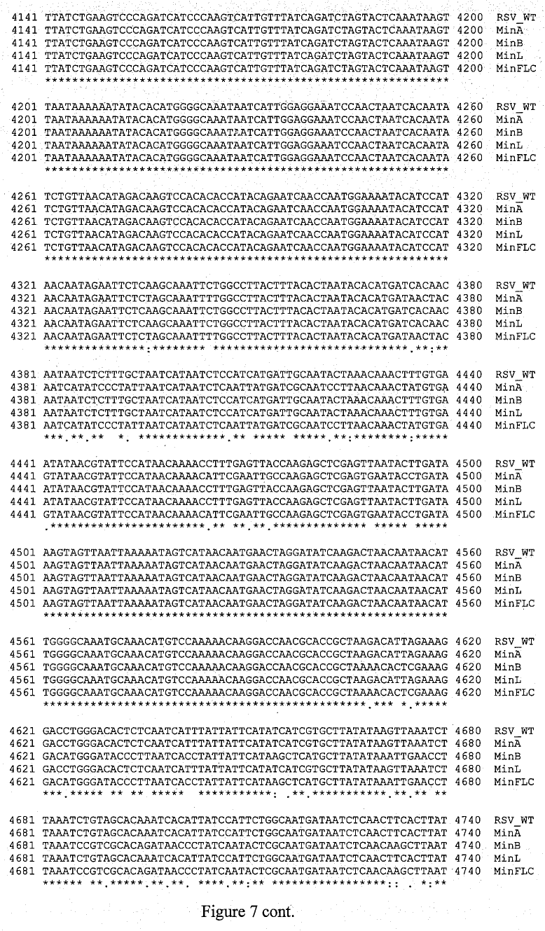

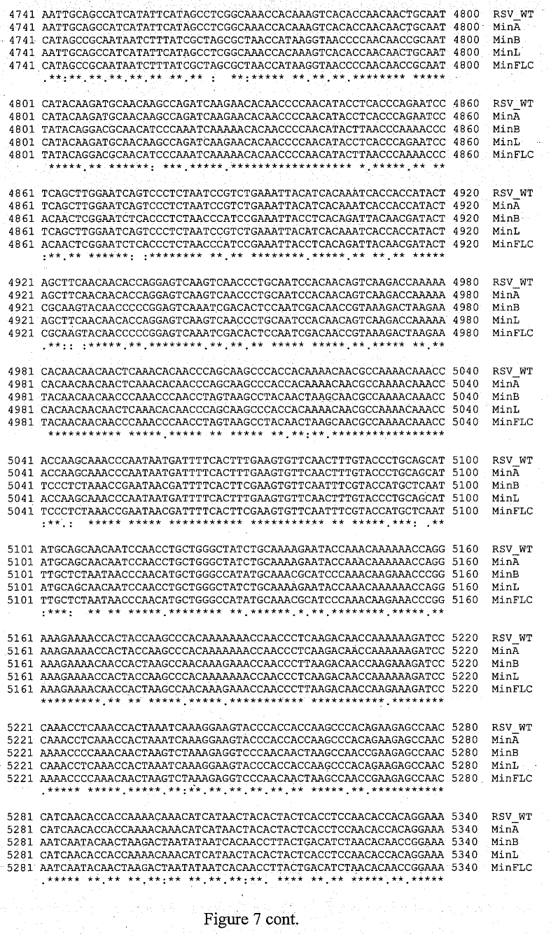

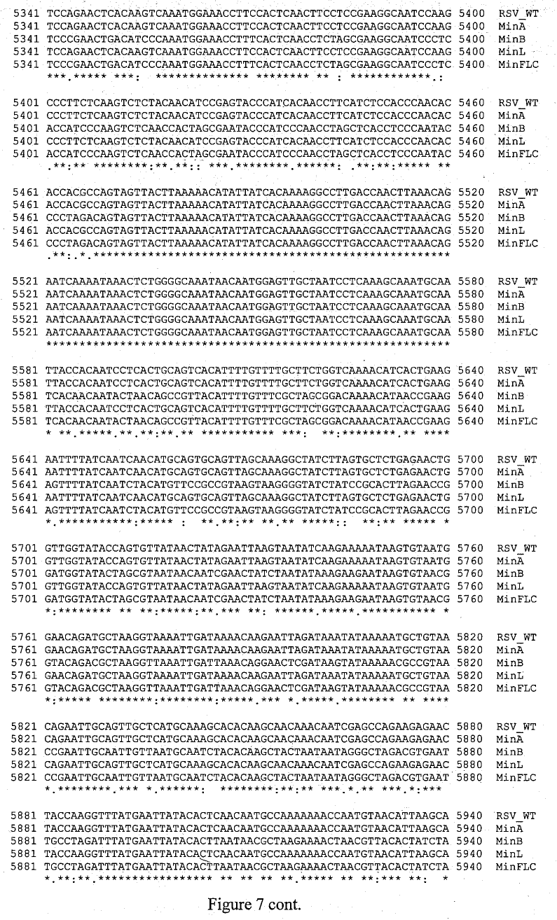

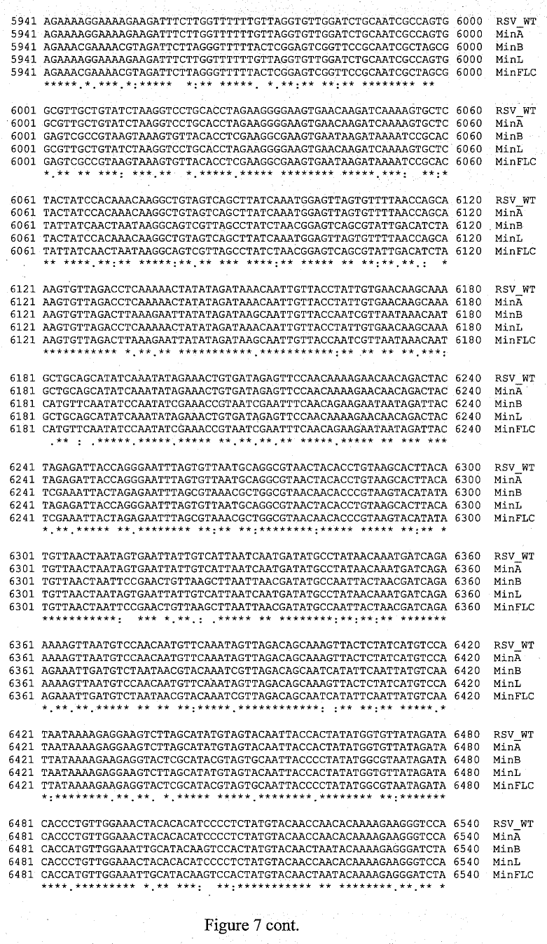

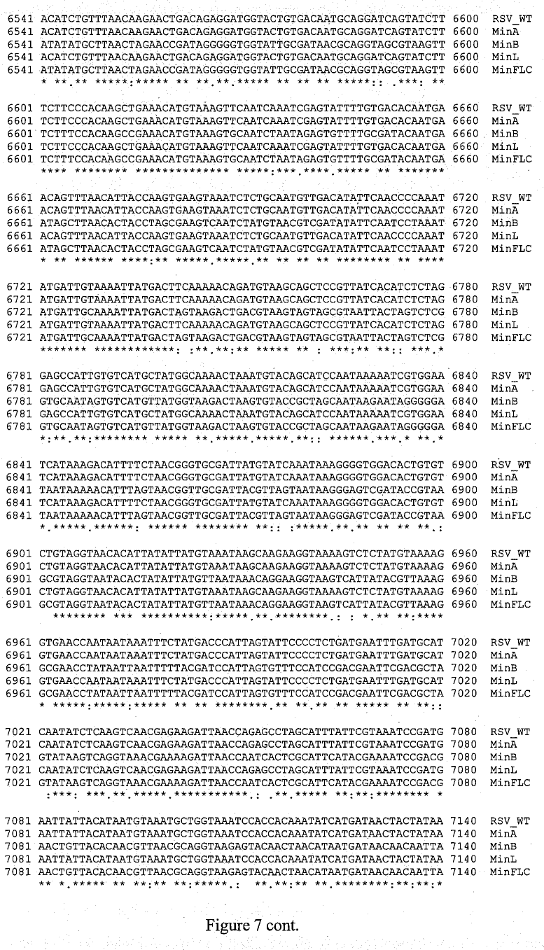

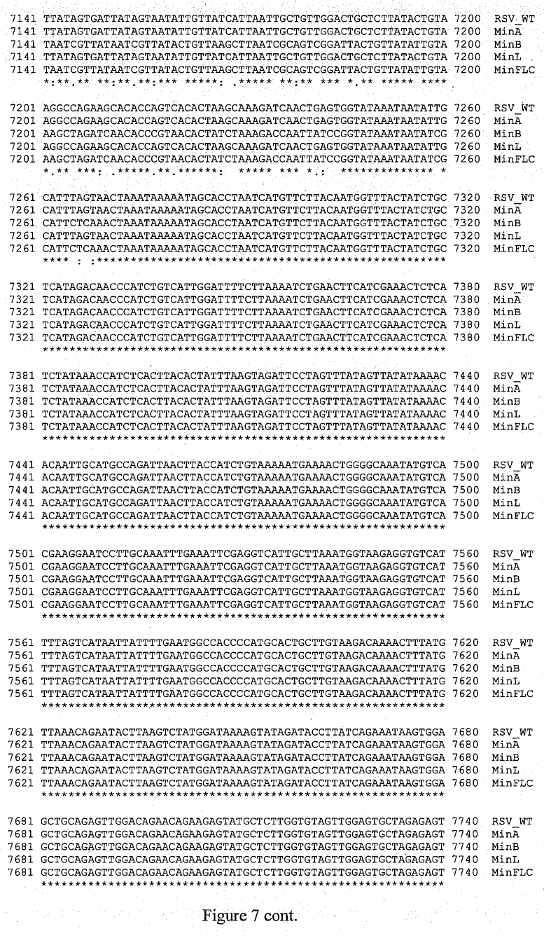

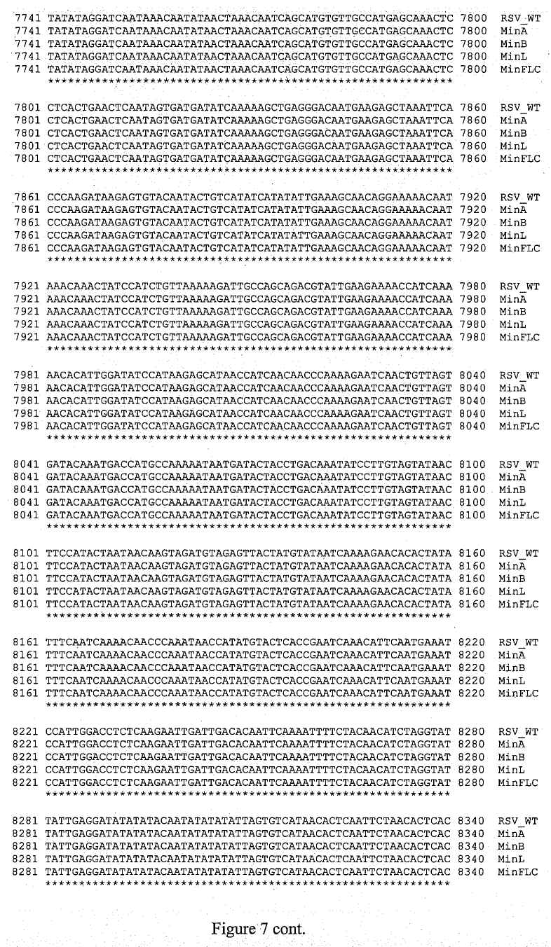

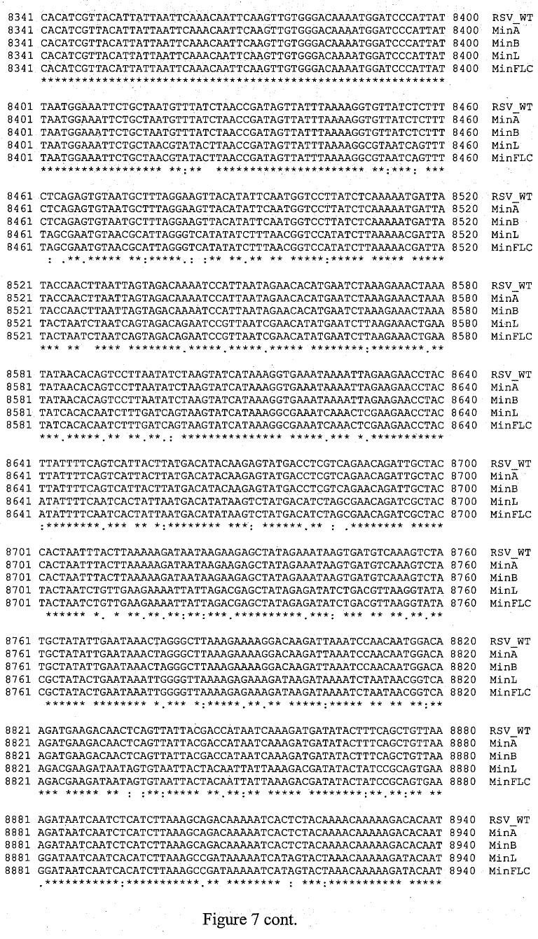

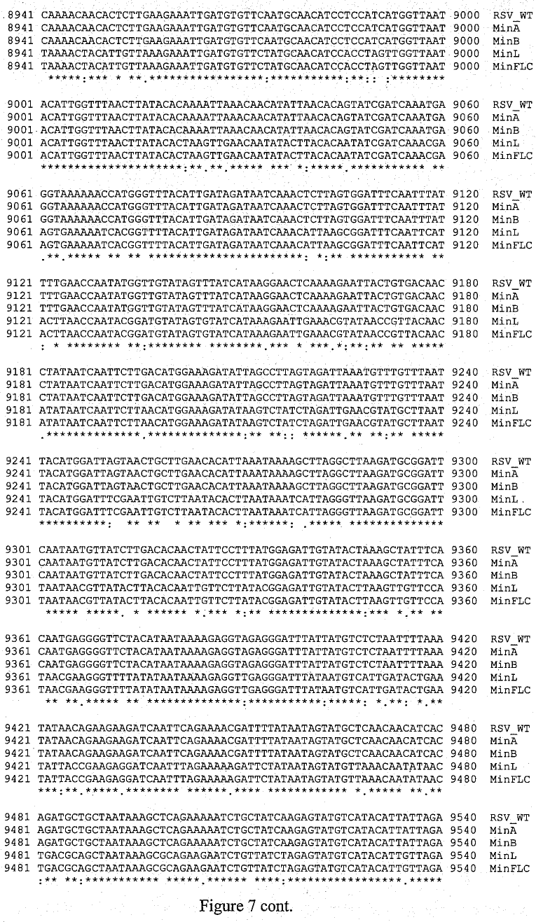

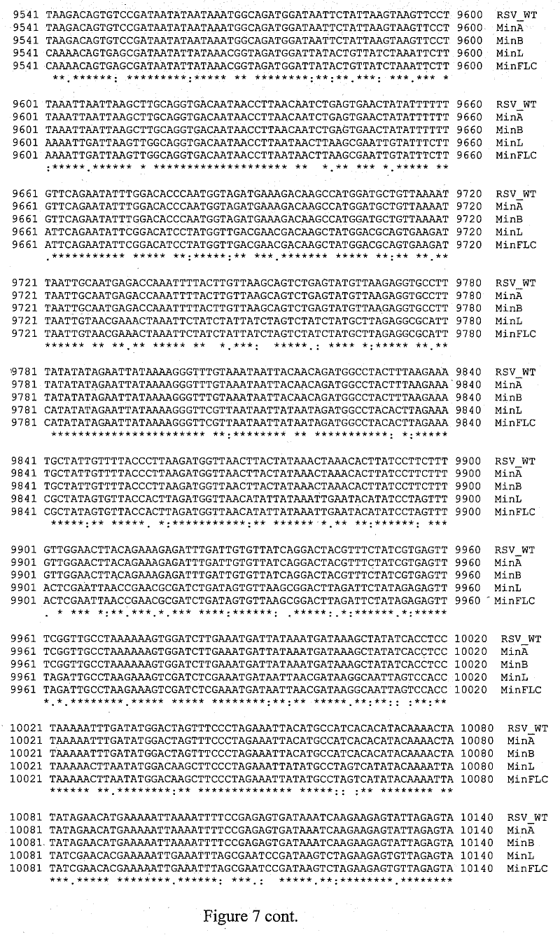

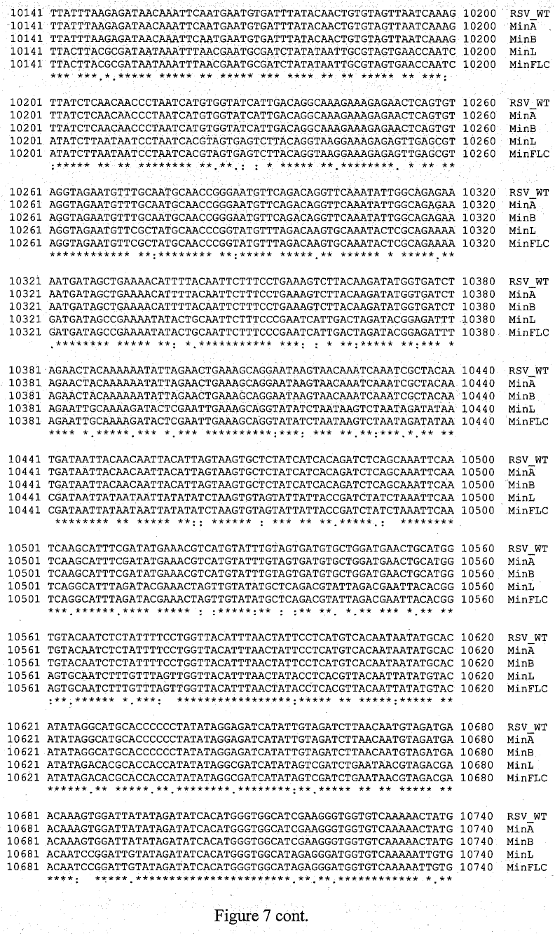

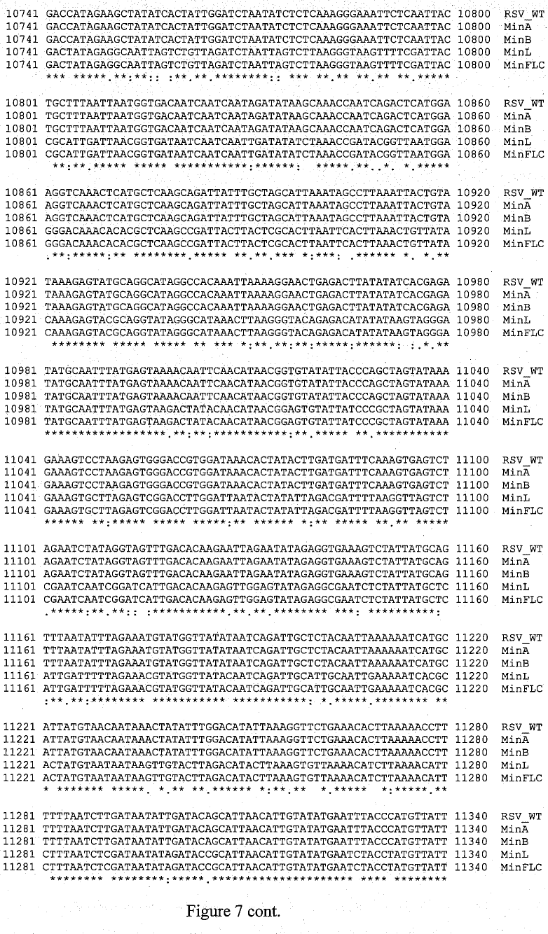

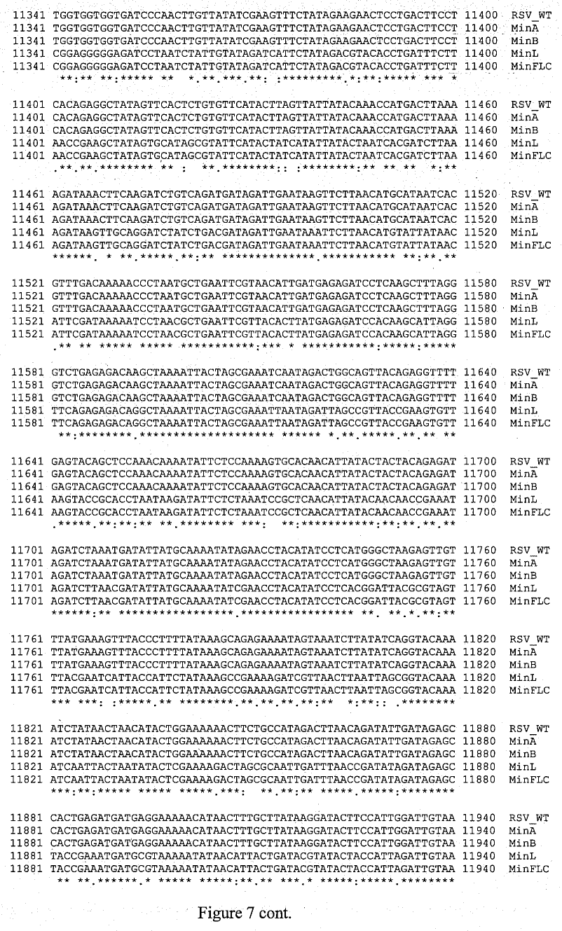

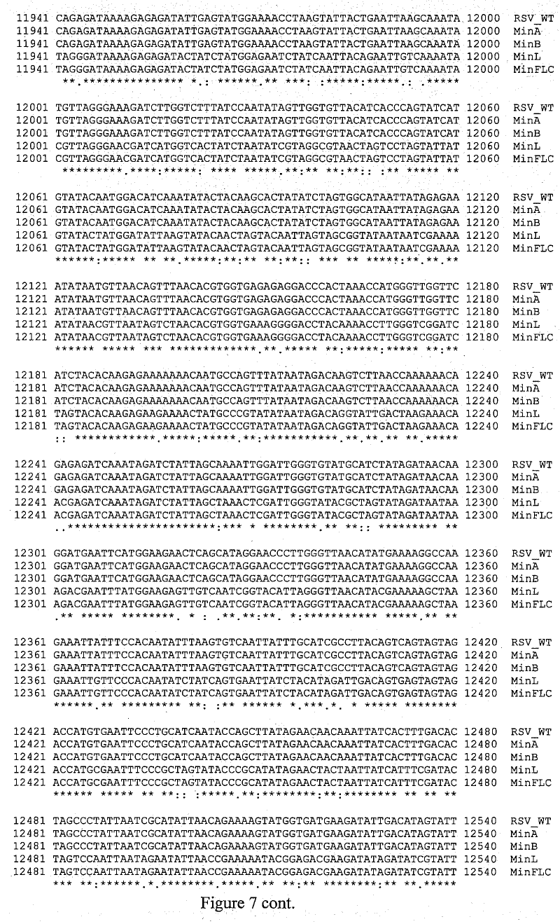

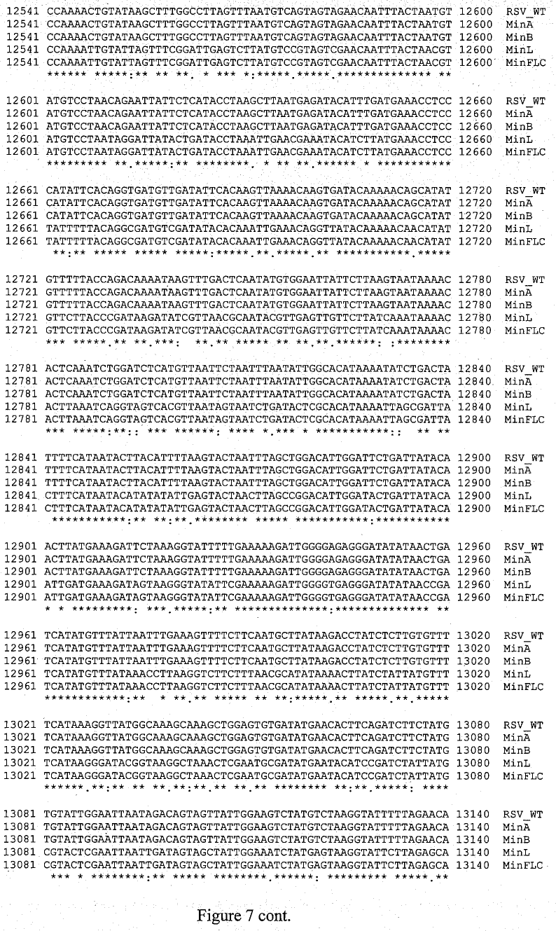

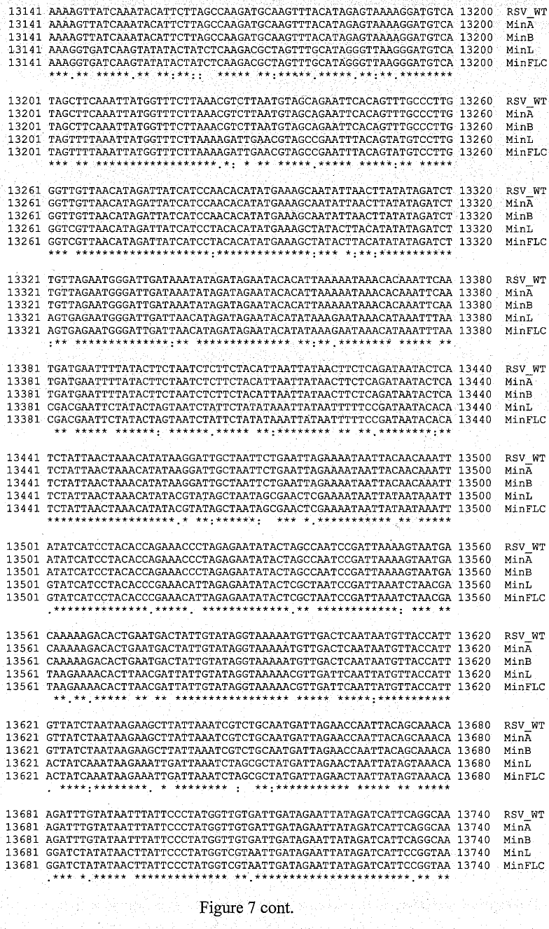

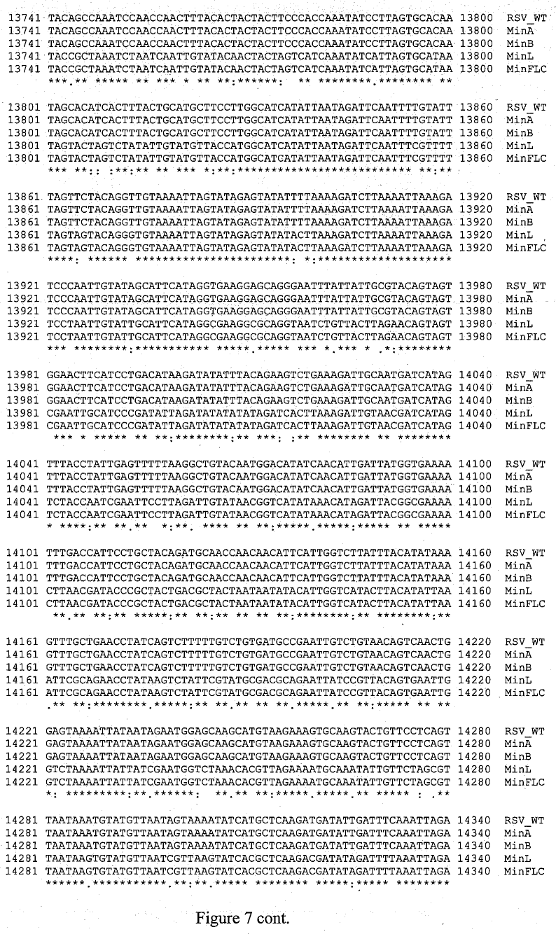

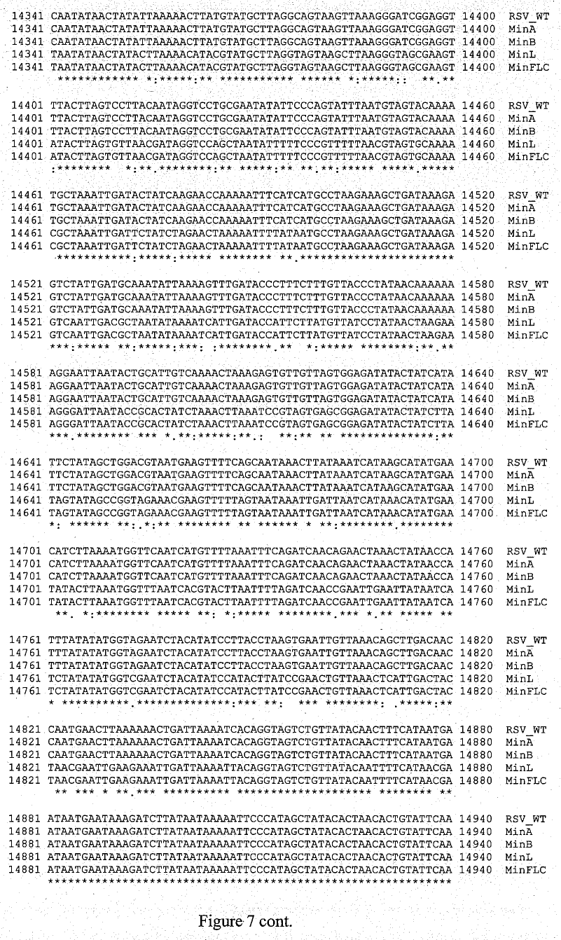

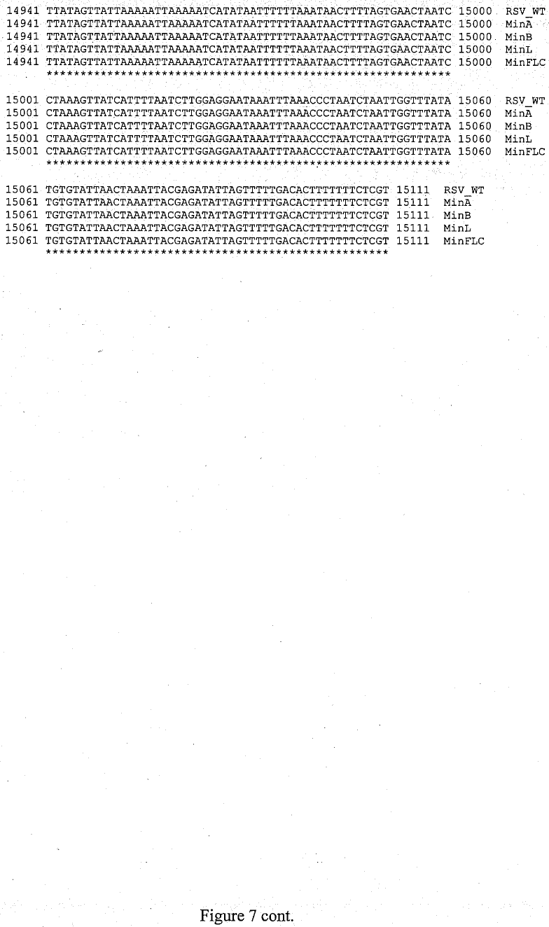

To further assess the characteristics of viruses having the mutations identified in the Min L replicate described in Example 8, one chimeric synthetic codon-pair deoptimized (CPD) rRSV (Min L) was generated based on the wt RSV backbone (Genbank Accession number M74568) that contained a CPD ORF of polymerase protein L. Variant forms of this virus were also created with the mutations identified in N (aa 136), P (aa 114) and M2-1 (aa 88) introduced in all possible combinations (FIG. 6A). Multi-cycle growth kinetics were then determined. In brief, confluent monolayer cultures of Vero cells in 6-well plates were infected in duplicate with rRSV, Min L or Min L containing the mutations of interest in all possible combinations at a multiplicity of infection (MOI) of 0.01 and incubated at 32 or 37° C. Viruses were harvested daily from day 1 to 14 by scraping infected cells into media followed by vortexing, clarification of the supernatant by centrifugation and freezing of virus aliquots. Each aliquot was titrated in duplicate at the permissive temperature of 32° C. For each time point, the mean value of the duplicate and standard deviation is shown (FIG. 6B). Plaque size phenotype on Vero cells at 32° C. of rRSV and CPD rRSVs is shown in FIG. 6C. Results indicating the temperature sensitivity of the codon-pair deoptimized RSVs are provided in Table 5.

TABLE 5
Temperature sensitivity of the codon-pair deoptimized
RSVs on Vero cells.
Virus titer (in log₁₀ PFU per ml) at indicated temperature (° C.) ^{a}

| Virus | 32 | 35 | 36 | 37 | 38 | 39 | 40 | T_{SH} ^{b} |
|---|---|---|---|---|---|---|---|---|
| Min L | 7.1 | 6.0 | 5.4 | 4.6 | 3.9 | <1 | <1 | 37 |
| Min L-N | 6.8 | 6.3 | 5.7 | 5.3 | 4.3 | 3.2 | <1 | 38 |
| Min L-P | 7.0 | 6.5 | 6.1 | 5.1 | 4.8 | 3.3 | <1 | 38 |
| Min L-M21 | 7.5 | 7.5 | 7.5 | 7.3 | 6.9 | 4.8 | <1 | 39 |
| Min L-NP | 6.8 | 6.7 | 6.3 | 5.8 | 4.9 | 3.8 | <1 | 39 |
| Min L-NM21 | 7.3 | 7.3 | 7.2 | 7.3 | 7.1 | 5.7 | <1 | 40 |
| Min L-PM21 | 7.4 | 7.4 | 7.5 | 7.3 | 7.0 | 6.4 | <1 | 40 |
| Min L-NPM21 | 7.2 | 7.1 | 7.2 | 7.1 | 6.9 | 6.7 | 3.2 | 40 |
| WT | 7.5 | 7.5 | 7.5 | 7.4 | 7.4 | 7.3 | 6.9 | >40 |

^{a} The ts phenotype for each virus was evaluated by plaque assay on Vero cells at the indicated temperatures. For viruses with a ts phenotype, the shut-off temperatures are marked (underlined). See footnote b for definition of shut-off temperature.
^{b} Shut off temperature (T_{SH}) is defined as the lowest restrictive temperature at which the reduction compared to 32° C. is 100-fold or greater than that observed for wt RSV at the two temperatures. The ts phenotype is defined as having a T_{SH} of 40° C. or less (bold).

What is claimed:

1. A recombinant polynucleotide encoding a respiratory syncytial virus (RSV) amino acid sequence of a parent RSV, comprising a nucleotide sequence that has from about 77% to about 93% identity with the corresponding nucleotide sequence of the parent virus genome.

2. The recombinant polynucleotide of claim 1, wherein the amino acid sequence of the parent RSV comprises an existing attenuating mutation.

3. The recombinant polynucleotide of claim 1, wherein the nucleotide sequence differing from the parent virus genome encodes any one of the RSV proteins NS1, NS2, N, P, M, SH, G, F, or L.

4. The recombinant polynucleotide of claim 3, wherein the nucleotide sequence encoding the RSV NS1 protein is from about 75% to about 95% identical to the corresponding sequence of the parent virus.

5. The recombinant polynucleotide of claim 4, wherein the nucleotide sequence encoding the RSV NS1 protein is about 87% identical to the corresponding sequence of the parent virus.

6. The recombinant polynucleotide of claim 5, wherein the nucleotide sequence encoding the RSV NS1 protein has the sequence of nucleotides 99 to 518 of SEQ ID NO: 5.

7. The recombinant polynucleotide of claim 3, wherein the nucleotide sequence encoding the RSV NS2 protein is from about 75% to about 95% identical to the corresponding sequence of the parent virus.

8. The recombinant polynucleotide of claim 7, wherein the nucleotide sequence encoding the RSV NS2 protein is about 88% identical to the corresponding sequence of the parent virus.

9. The recombinant polynucleotide of claim 8, wherein the nucleotide sequence encoding the RSV NS2 protein has the sequence of nucleotides 628 to 1002 of SEQ ID NO: 5.

10. The recombinant polynucleotide of claim 3, wherein the nucleotide sequence encoding the RSV N protein is from about 70% to about 90% identical to the corresponding sequence of the parent virus.

11. The recombinant polynucleotide of claim 10, wherein the nucleotide sequence encoding the RSV N protein is about 80% identical to the corresponding sequence of the parent virus.

12. The recombinant polynucleotide of claim 11, wherein the nucleotide sequence encoding the RSV N protein has the sequence of nucleotides 1141 to 2316 of SEQ ID NO: 5.

13. The recombinant polynucleotide of claim 3, wherein the nucleotide sequence encoding the RSV P protein is from about 75% to about 95% identical to the corresponding sequence of the parent virus.

14. The recombinant polynucleotide of claim 13, wherein the nucleotide sequence encoding the RSV P protein is about 84% identical to the corresponding sequence of the parent virus.

15. The recombinant polynucleotide of claim 14, wherein the nucleotide sequence encoding the RSV P protein has the sequence of nucleotides 2347 to 3072 of SEQ ID NO: 5.

16. The recombinant polynucleotide of claim 3, wherein the nucleotide sequence encoding the RSV M protein is from about 75% to about 95% identical to the corresponding sequence of the parent virus.

17. The recombinant polynucleotide of claim 16, wherein the nucleotide sequence encoding the RSV M protein is about 83% identical to the corresponding sequence of the parent virus.

18. The recombinant polynucleotide of claim 17, wherein the nucleotide sequence encoding the RSV M protein has the sequence of nucleotides 3262 to 4032 of SEQ ID NO: 5.

19. The recombinant polynucleotide of claim 3, wherein the nucleotide sequence encoding the RSV SH protein is from about 85% to about 95% identical to the corresponding sequence of the parent virus.

20. The recombinant polynucleotide of claim 19, wherein the nucleotide sequence encoding the RSV SH protein is about 92% identical to the corresponding sequence of the parent virus.

21. The recombinant polynucleotide of claim 20, wherein the nucleotide sequence encoding the RSV SH protein has the sequence of nucleotides 4304 to 4498 of SEQ ID NO: 5.

22. The recombinant polynucleotide of claim 3, wherein the nucleotide sequence encoding the RSV G protein is from about 65% to about 85% identical to the corresponding sequence of the parent virus.

23. The recombinant polynucleotide of claim 22, wherein the nucleotide sequence encoding the RSV G protein is about 78% identical to the corresponding sequence of the parent virus.

24. The recombinant polynucleotide of claim 23, wherein the nucleotide sequence encoding the RSV G protein has the sequence of nucleotides 4577 to 5473 of SEQ ID NO: 5.

25. The recombinant polynucleotide of claim 3, wherein the nucleotide sequence encoding the RSV F protein is from about 65% to about 85% identical to the corresponding sequence of the parent virus.

26. The recombinant polynucleotide of claim 25, wherein the nucleotide sequence encoding the RSV F protein is about 77% identical to the corresponding sequence of the parent virus.

27. The recombinant polynucleotide of claim 26, wherein the nucleotide sequence encoding the RSV F protein has the sequence of nucleotides 5550 to 7274 of SEQ ID NO: 5.

28. The recombinant polynucleotide of claim 3, wherein the nucleotide sequence encoding the RSV L protein is from about 70% to about 90% identical to the corresponding sequence of the parent virus.

29. The recombinant polynucleotide of claim 28, wherein the nucleotide sequence encoding the RSV L protein is about 79% identical to the corresponding sequence of the parent virus.

30. The recombinant polynucleotide of claim 29, wherein the nucleotide sequence encoding the RSV L protein has the sequence of nucleotides 8387 to 14884 of SEQ ID NO: 5.

31. A recombinant polynucleotide encoding at least two RSV proteins having amino acid sequences of a parent RSV, comprising separate nucleotide sequences encoding each of the RSV proteins that have from about 77% to about 93% identity with the corresponding nucleotide sequence of the parent virus genome.

32. The recombinant polynucleotide of claim 31, wherein the nucleotide sequences encoding each parent RSV protein, comprise any two recombinant polynucleotides encoding different RSV proteins selected from: NS1, NS2, N, P, M, SH, G, F, and L.

33. The recombinant polynucleotide of claim 31, wherein the nucleotide sequences encoding each parent RSV protein, comprise any three recombinant polynucleotides encoding different RSV proteins selected from: NS1, NS2, N, P, M, SH, G, F, and L.

34. The recombinant polynucleotide of claim 31, wherein the nucleotide sequences encoding each parent RSV protein, comprise any four recombinant polynucleotides encoding different RSV proteins selected from: NS1, NS2, N, P, M, SH, G, F, and L.

35. The recombinant polynucleotide of claim 31, wherein the nucleotide sequences encoding each parent RSV protein, comprise any five recombinant polynucleotides encoding different RSV proteins selected from: NS1, NS2, N, P, M, SH, G, F, and L.

36. The recombinant polynucleotide of claim 31, wherein the nucleotide sequences encoding each parent RSV protein, comprise any six recombinant polynucleotides encoding different RSV proteins selected from: NS1, NS2, N, P, M, SH, G, F, and L.

37. The recombinant polynucleotide of claim 31, wherein the nucleotide sequences encoding each parent RSV protein, comprise any seven recombinant polynucleotides encoding different RSV proteins selected from: NS1, NS2, N, P, M, SH, G, F, and L.

38. The recombinant polynucleotide of claim 31, wherein the nucleotide sequences encoding each parent RSV protein, comprise any eight recombinant polynucleotides encoding different RSV proteins selected from: NS1, NS2, N, P, M, SH, G, F, and L.

39. The recombinant polynucleotide of claim 31, wherein the nucleotide sequences encoding each parent RSV protein, comprise any nine recombinant polynucleotides encoding different RSV proteins selected from: NS1, NS2, N, P, M, SH, G, F, and L.

40. A recombinant RSV genome or antigenome comprising the genome encoded by pRSV_Min A, pRSV_Min B, pRSV_Min L, pRSV_Min FLC, pRSV_Min L_N, pRSV_Min L_p, pRSV_Min L_M21, pRSV_Min L_NP, pRSV_Min L_NM21, pRSV_Min L_PM21, or pRSV_Min L_NPM21.

41. A recombinant RSV genome or antigenome comprising a nucleotide change in one or more of the following:
a. a change in the codon encoding amino acid 136 of the N protein,
b. a change in the codon encoding amino acid 114 of the P protein, or
c. a change in the codon encoding amino acid 88 of the M2-1 protein.

42. The recombinant RSV genome or antigenome of claim 52, wherein any one of said nucleotide changes causes an amino acid other than:
a. lysine to be encoded at position 136 of the N protein,
b. glutamic acid to be encoded at position 114 of the P protein, or
c. asparagine to be encoded at position 88 of the M2-1 protein.

43. The recombinant RSV genome or antigenome of claim 53, wherein arginine is encoded at position 136 of the N protein, valine is encoded at position 114 of the P protein, and/or lysine is encoded at position 88 of the M2-1 protein.

44. A method of producing a recombinant RSV, comprising expressing the genome or antigenome of claim 40 in a cell.

45. A method of producing an immune response to a viral protein, comprising administering the recombinant RSV of claim 40 to an animal.

46. The method of claim 45, wherein the RSV is administered via injection, aerosol delivery, nasal spray, nasal droplets, oral inoculation, or topical application.

47. The method of claim 45, wherein the animal is a mammal.

48. The method of claim 47, wherein the mammal is a human.

49. The recombinant RSV of claim 40, wherein said virus is attenuated.

50. The recombinant RSV of claim 49, wherein the genome or antigenome is modified to include a previously characterized RSV mutation or deletion.

51. A vaccine composition comprising the recombinant RSV of claim 40.
